(19) United States
(12) Patent Application Publication
(10) Pub. No.: US 2024/0166637 A1
(43) Pub. Date: May 23, 2024

(54) COMPOUNDS AND METHODS FOR TREATING MALARIA

(71) Applicants: Microbiotix, Inc., Worcester, MA (US); The United States Of America As Represented By The Secretary, Department of Health and Human Service, Bethesda, MD (US)

(72) Inventors: Samanthi L. Waidyarachchi, Worcester, MA (US); Son T. Nguyen, Wayland, MA (US); Xiaoyuan Ding, Natick, MA (US); Sharmila Adhikari, Billerica, MA (US); John D. Williams, Glenwood, MD (US); Norton P. Peet, Holland, MI (US); Zachary D. Aron, Acton, MA (US); Sanjay A. Desai, Potomac, MD (US); Michelle M. Butler, Auburn, MA (US)

(73) Assignees: Microbiotix, Inc., Worcester, MA (US); The United States Of America As Represented By The Secretary, Department of Health and Human Service, Bethesda, MD (US)

(21) Appl. No.: 18/272,845

(22) PCT Filed: Jan. 21, 2022
(86) PCT No.: PCT/US2022/013223

§ 371 Date: Jul. 18, 2023

Related U.S. Application Data
(60) Provisional application No. 63/140,308, filed on Jan. 22, 2021.

(51) Int. Cl. C07D 409/14; A61K 45/06; A61P 33/06; C07D 405/14; C07D 413/14; C07D 417/14

(52) U.S. Cl. CPC C07D 409/14; A61K 45/06; A61P 33/06; C07D 405/14; C07D 413/14; C07D 417/14

(57) ABSTRACT

The present invention is related to the development of novel compounds and methods for the treatment and/or prevention of malaria. The compounds prevent the formation by the malaria parasite of the plasmodium surface anion channel (PSAC) on the surface of the host cell. The compounds and methods described herein are effective against infection by a wide variety of Plasmodia strains known as the causative agent of malaria.

### CROSS-REFERENCE TO RELATED APPLICATIONS

This application is a United States national stage filing under 35 U.S.C. § 371 of international (PCT) application no. PCT/US2022/013223, filed Jan. 21, 2022, and designating the US, which claims priority to U.S. Provisional Appin. No. 63/140,308 filed Jan. 22, 2021.

### STATEMENT REGARDING FEDERALLY SPONSORED RESEARCH

This invention was made with government support under NIH grant R44 AI100339. The United States Government has certain rights in the invention.

### FIELD OF THE INVENTION

The present invention is directed to the discovery of a novel class of pyridazinone compounds effective for the treatment and prevention of malaria. The compounds of the present invention are capable of inhibiting or otherwise interfering with the ability of the malaria parasite to form the plasmodial surface anion channel (PSAC) with the host cell. As such, the compounds described herein are capable of treating or preventing malaria caused by a number of Plasmodium species.

### BACKGROUND OF THE INVENTION

Malaria remains one of the world's most formidable health challenges and one of the few diseases for which death and infection continue to be measured in hundreds of millions of lives each year (Mira-Martinez et al., Cell Microbiol., 15:1913-1923 (2013)). In 2016, there were 216 million malaria cases that led to 445,000 deaths. Of these, roughly two thirds (290,000) were children under five years of age with the bulk of these deaths occurring in sub-Saharan Africa (Pillai et al., Mol. Pharmacol., 82:1104-1114 (2012)). Malaria is caused by the protozoan parasite genus Plasmodium, and the two major species, P. falciparum and P. vivax, account for virtually the entire global malaria burden. Malaria is transmitted to humans via the bite of the infected female Anopheles mosquito. Inside the body, Plasmodium parasites multiply in the liver and the bloodstream before being transmitted back to the mosquito vector when an infected human is bitten (Pillai et al., Mol. Pharmacol., 77:724-733 (2010)). The disease presents as a febrile illness, with symptoms commencing 10-15 days after the original bite. Symptoms include high fever, chills, abdominal pain, headaches, nausea, and vomiting. The acute phase lasts from 4-6 hours and cycles every 1-3 days (Pillai et al., supra (2010)). Prevention of infections and therapy are the only options for reducing the morbidity and mortality.

The life cycle of Plasmodium species cycles through several stages. Initially, the mosquito vector transmits the Plasmodium parasite in the sporozoite stage to the host (human) during a blood meal. Shortly thereafter (within 60 minutes), sporozoites invade liver cells, where they replicate and divide as merozoites. The infected liver cell ruptures, releasing the merozoites into the bloodstream where they invade red blood cells and begin the asexual reproductive stage, which is the symptomatic stage of the disease. Symptoms develop 4-8 days after the initial red blood cell invasion and lasts 36-72 hours (from red blood cell invasion to hemolysis).

Plasmodium vivax and Plasmodium ovale can also enter a dormant state in the liver, the hypnozoite. Merozoites released from red blood cells can invade other red blood cells and continue to replicate, or in some cases, they differentiate into male or female gametocytes. Gametocytes concentrate in skin capillaries and are then taken up by the mosquito vector in another blood meal. In the gut of the mosquito, each male gametocyte produces eight microgametes after three rounds of mitosis; the female gametocyte matures into a macrogamete. Male microgametes are motile forms with flagellae and seek the female macrogamete. The male and female gametocytes fuse, forming a diploid zygote, which elongates into an ookinete; this motile form exits from the lumen of the gut across the epithelium as an oocyst. Oocysts undergo cycles of replication and form sporozoites, which move from the abdomen of the mosquito to the salivary glands. Thus, 7-10 days after the mosquito feeds on blood containing gametocytes, it may be ‘armed’ and able to infect another human with Plasmodium spp. with her bite.

Drugs that prevent Plasmodium spp. invasion or proliferation in the liver have prophylactic activity, drugs that block the red blood cell stage are required for the treatment of the symptomatic phase of the disease, and compounds that inhibit the formation of gametocytes or their development in the mosquito (including drugs that kill mosquitoes feeding on blood) are transmission-blocking agents.

Malaria prevention is achieved by both physical and chemical means. Preventing malaria in vulnerable populations is also a priority, especially given the lack of successful vaccine development (Anthony et al., Malar. J., 11:316 (2012)). According to WHO, between 2014 and 2016, an estimated 582 million insecticidal nets (ITNs) were delivered to endemic countries, a major increase over the 350 million nets delivered from 2011-2013 (Pillai et al., supra (2012)). In 2016, 2.9% of the global population at risk of malaria were protected by indoor residual spraying worldwide. However, resistance to pyrethroids, the only insecticide class currently used in ITNs, is widespread, with 81% of malaria endemic countries reporting pyrethroid resistance in 2016 (Pillai et al., supra (2012)).

Options and methods for treating malaria are becoming increasing difficult due to the development of drug resistance. The number of available antimalarial drugs is surprisingly small given the large burden of disease and the resulting incidence of mortality exacted by malaria (Phillips et al., Nat. Rev. Dis. Primers, 3:17050 (2017)). Chloroquine (CQ), a derivative of the original aminoalcohol malaria drug, quinine, has been the mainstay of malaria chemotherapy for much of the past five decades (Kutner et al., Biochim. Biophys. Acta, 687:113-117 (1982)). It has several advantages including limited toxicity, ease of use, low cost, and efficient synthesis (Kutner et al., supra (1982)). Unfortunately, the use of CQ for treating malaria caused by P. falciparum has been largely hampered due to reduced parasite sensitivity to the drug (Anthony et al., supra (2012)). Currently, artemisinin-based combination therapy (ACT) (Desai, S., and A. Pillai, “Inhibitors Of The Plasmodial Surface Anion Channel As Antimalarials”, U.S. Pat. No. 8618090, filed Jul. 15, 2009 and issued Dec. 31, 2013; Dondorp et al., N. Engl. J. Med., 361:455-467 (2009); Duraisingh, M. T. and A. F. Cowman, Acta Trop., 94:181-190 (2005)) is the WHO-recommended standard of care against P. falciparum malaria and involves a double or triple-combination therapy composed of an artemisinin derivative, such as artemether, artesunate, or dihydroartemisinin, combined with a partner drug such as lumefantrine, amodiaquine, or mefloquine (See Table 1 for an overview of approved treatments for malaria) (Pillai et al., supra (2012)). ACT is geared towards circumventing, or at least delaying, resistance development (Kirk et al., FEBS Lett., 323:123-128 (1993)). Unfortunately, to date, several instances of reduced susceptibility to ACT have been reported in Southeast Asia (Desai, S., “Plasmodial Surface Anion Channel Inhibitors For The Treatment Or Prevention Of Malaria”, U.S. Pat. No. 9,320,786, filed Apr. 11, 2012, and issued Apr. 26, 2016; Marshall et al., Growth Regul., 5:69-84 (1995); Nguitragool et al., Cell, 145:665-677 (2011)).

TABLE 1
Malaria drugs in clinical use - WHO Treatment Guidelines
- (from Guidelines for the Treatment of Malaria 3^{rd} ed.)

| Clinical diagnosis/ | |
|---|---|
| Plasmodium species | Recommended Drugs |
| Treatment of uncomplicated P. | Artemether + lumefantrine |
| falciparum malaria | Artesunate + amodiaquine |
| | Artesunate + mefloquine |
| | Dihydroartemisinin + piperaquine |
| | Artesunate + sulfadoxine-pyrimethamine (SP) |
| Treatment of uncomplicated P. | Quinine + clindamycin |
| falciparum malaria in special risk | An ACT at the same target dose as 5 kg children |
| groups: | Avoid artesunate + SP if receiving co-trimoxazole; |
| First trimester pregnancy | avoid artesunate + amodiaquine if receiving efavirenz |
| Infants (<5 kg) | or zidovudine |
| HIV co-infection | ACT |
| Non-immune travelers | ACT and closely monitored |
| Uncomplicated | |
| hyperparasitemia | |
| Treating uncomplicated P. vivax, | Treat as for uncomplicated P. falciparum malaria |
| P. ovale, P. malariae or P. | ACT or chloroquine (except first trimester pregnancy) |
| knowlesi malaria | ACT (except first trimester pregnancy) |
| Blood stage infection | Quinine |
| Unknown species | |
| In areas w/chloroquine- | |
| susceptible infections | |
| In areas w/chloroquine- | |
| resistant infections | |
| First trimester pregnancy w/ | |
| chloroquine-resistant P. vivax | |
| Preventing relapse in P. vivax or | Primaquine (14 days) |
| P. ovale malaria | Primaquine (8 weeks w/close medical supervision) |
| Children and adults | Assess risk/benefits of primaquine |
| G6PD deficiency | Chloroquine until delivery/breastfeeding complete - |
| G6PD status unknown | then primaquine on basis of G6PD status |
| Pregnant/breast feeding | |
| women | |
| Treating severe malaria | Artesunate (IV or IM) for 24 hours followed by ACT |
| Adults and children (all) | (3 days) when oral therapy tolerable |
| | If parenteral artesunate is unavailable, use artemether |
| | in preference to quinine |
| Treating suspected severe malaria | Artesunate (IM) if available; if not, artemether (IM); if |
| (pre-referral treatment | not, quinine (IM) - then refer to appropriate higher- |
| Adults and children | level facility |
| Children <6 years old | If artesunate (IM) is unavailable, artesunate (rectal) |

Therefore, extensive spread of drug resistance involving classic antimalarial drugs necessitates a search for promising compounds, preferably with new chemical scaffolds and novel antimalarial targets (Alkhalil et al., Blood, 104:4279-4286 (2004); Pillai et al., Mol. Microbiol., 88:20-34 (2013)). However, the antimalarial pipeline is inadequate. The compounds listed in Table 1 are the current competition if a new antimalarial were to be approved today. However, because resistance to chloroquine and its analogs as well as the artemisinins is becoming more frequent, new antimalarial compounds need to be discovered. Table 2 shows the drugs that are in the current global clinical trial pipeline listed in order of life cycle target and then by phase of trial development. Those in preclinical development are not listed but can be found in the literature (Dondorp et al., N. Engl. J. Med., 361:455-467 (2009); Muregi, F. W. and A. Ishis, Drug Devel. Res., 71:20-32 (2010)). The majority of the compounds in the pipeline target the asexual blood stage of infection. Several also target disease transmission, chemoprotection, and infection relapse. Although the combination of current options plus the clinical pipeline appear to be robust, many of the drugs are analogs or combinations of existing drugs or have been withdrawn due to inadequate efficacy or safety. In addition, considering the need for combinations to treat even drug-sensitive malaria, the current pipeline for new antimalarial drugs is not adequate to meet the needs of eradicating the disease. Clearly, new agents with novel mechanisms that are refractory to resistance development are needed to overcome developing resistance to all current agents.

TABLE 2
The clinical pipeline of antimalarials.

| Compound (Sponsor) | Clinical Phase | Life cycle target |
|---|---|---|
| Artemisone (HKUST) | Withdrawn | Asexual blood stages |
| SJ733 (St Jude, Eisai and | 1 | Asexual blood stages |
| MMV) | | |
| CDRI 9778 (CDRI, Ipca Labs) | 1 | Asexual blood stages |
| ACT-451840 (Actelion) | 1 | Asexual blood stages |
| N-tert butyl isoquine (LSTM, | 1 | Asexual blood stages |
| GSK) | | |
| SAR97276 (Sanofi) | 2 | Asexual blood stages |
| AQ13 (Immtech) | 2 | Asexual blood stages |
| Fosmidomycin-piperaquine | 2 | Asexual blood stages |
| (Jomaa Pharma and GmbH) | | |
| Co-trimoxazole (ITM | 3 | Asexual blood stages |
| Antwerp) | | |
| Artemisinin-naphthoquine | 4 | Asexual blood stages |
| (Kunming Pharma) | | |
| Cipargamin [KAE609] | 2 | Asexual blood stages, |
| (Novartis, MMV) | | transmission |
| | | reduction |
| Methylene Blue-Amodaquine | 2 | Asexual blood stages, |
| (Heidelberg U.) | | transmission |
| | | reduction |
| Artefenomel (OZ439)- | 2 | Asexual blood stages, |
| Ferroquine (Sanofi, MMV) | | transmission |
| | | reduction |
| Tafenoquine (GSK and MMV; | 3 | Chemoprotection and relapse |
| GSK and US Army) | | prevention |
| P218 (Janssen, Biotec | 1 | Asexual blood stages and |
| Thailand) | | chemoprotection |
| DSM265 (Takeda, MMV; | 2 | Asexual blood stages and |
| UTSW, UW and Monash U.) | | chemoprotection |
| MMV048 (MMV and UCT) | Withdrawn | Asexual blood stages, |
| | | chemoprotection and transmission |
| | | reduction |
| KAF156 (Novartis, MMV) | 2 | Asexual blood stages, |
| | | chemoprotection and transmission |
| | | reduction |
| Sevuparin- | 1-2 | Host related (release parasite- |
| atovaquone/proquanil (Modus | | infected red blood cells into |
| Ther.) | | circulation) |

### The Plasmodial Surface Anion Channel

The Plasmodial Surface Anion Channel (PSAC) is a novel, conserved, and essential target in Plasmodium. PSAC is essential to parasite survival and represents an antimalarial drug target that has not been previously exploited (Overman, R., Am. J. Physiol., 152:113-121 (1948); PATH, Stayin the Course? Malaria Research and Development in a Time of Economic Uncertainty, Program for Appropriate Technology in Health, Seattle (2011)). The presence of PSAC provides a mechanistic explanation for the long known increased permeability of infected erythrocytes to diverse solutes (Hooft van Huijsduijnen, R., and T. N. Wells, Curr. Opin. Pharmacol., 42:1-6 (2018); Ito et al., eLIFE, 6:e23485 (2017); Maude et al., Drug Devel. Res., 71:12019 (2010)). This channel localizes to the erythrocyte membrane and is exposed to host plasma. Studies using nutrient restriction and specific inhibitors indicate that PSAC plays an essential role in parasite nutrient acquisition of essential amino acids, sugars, purines, and some vitamins (Overman, R., supra (1948)). Ion permeation through this shared ion channel also leads to dramatic changes in host erythrocyte cation concentrations (Noedl et al., N. Engl. J. Med., 359:2619-2620 (2008)). In recent years, genetic mapping and molecular transfections have demonstrated that three proteins encoded by the parasite, CLAG3, RhopH2, and RhopH3, make up the structural components of the PSAC (Duraisingh, M. T. and A. F. Cowman, Acta Trop., 94:181-190 (2005); Krogstad et al., Science, 238:1283-1285 (1987); Lisk, G., and S. A. Desai, Eukaryot. Cell, 4:2153-2159 (2005); Phyo et al., Lancet, 379:1960-1966 (2012)). Consistent with an essential role in parasite biology, both PSAC activity and each of these subunit proteins are stringently conserved with ≥92% identity in sequences of each protein amongst P. falciparum lines from all three endemic continents (Duraisingh, M. T. and A. F. Cowman, supra (2005); Phyo et al., supra (2012)). While clag3 has between 2 to 5 closely related paralogs in each Plasmodium spp., rhoph2 and rhoph3 are single-copy genes in each species and cannot be disrupted, providing molecular validation of the PSAC target.

The three subunits have no homology to known mammalian ion channels, accounting for PSAC's unusual functional properties and suggesting that specific drugs that do not block human transporters can be developed. PSAC represents a novel antimalarial target not yet exploited by available antimalarial drugs (Overman, R. R., supra (1948)). Dr. Sanjay Desai and his NIAID team have developed and used high-throughput screening (HTS) technologies to find potent PSAC inhibitors. The screening methods yield estimated inhibitory affinities (K_{0.5} values) that match those measured with single channel patch clamp (PATH, supra (2011)), the accepted standard for ion channels. The channel's exposed location on the host membrane ensures direct access by soluble inhibitors in plasma, and it eliminates active drug extrusion from the cell as a possible mechanism of acquired resistance. Extrusion of unmodified drug from infected cells has plagued many antimalarials, accounting for resistance to agents such as chloroquine, mefloquine, and possibly artemisinin (Desai, S., and A. Pillai, “Inhibitors Of The Plasmodial Surface Anion Channel As Antimalarials”, U.S. Pat. No. 8618090, filed Jul. 15, 2009 and issued Dec. 31, 2013; Houston, J. B., Biochem. Pharmacol., 47:1469-1479 (1994)). The strongly conserved primary sequences of the PSAC proteins, their requirement for co-translational assembly, and complex trafficking to the host membrane all implicate a highly constrained target and suggest that resistance mutations may not arise easily. Novel antimalarial selected from high-throughput screening (HTS) campaign. The critical role that PSAC plays in malaria infections was not known until a HTS of the target was conducted in the laboratory of Dr. Sanjay Desai, NIAID (PATH, supra (2011)). The screening assay tested the ability of compounds to prevent sorbitol-induced PSAC-mediated osmotic lysis of P. falciparum-infected erythrocytes. Approximately 70,000 compounds from synthetic and natural product libraries were screened, to reveal inhibitors from multiple structural classes. Single-channel patch-clamp studies indicated that these compounds act directly on PSAC, further supporting a proposed role for PSAC in the transport of solutes such as sorbitol, other sugars, anions and amino acids (PATH, supra (2011)). Of special interest is one chemical scaffold (Overman, R. R., supra (1948)), represented by ISG-21 (Desai, S., U.S. Pat. No. 9,320,786, supra); Bevan et al., Anal. Chem., 72:1781-1787 (2000)), shown in Tables 3 and 4 below, which displayed promising potency in vitro.

The ISG-21 compound displays potent PSAC inhibitory activity with nanomolar K_{0.5} values (Table 3). It also kills parasites at concentrations near or slightly higher than those required to block the channel, consistent with a single mechanism of action. ISG-21 displayed low cytotoxicity in a 3-day HeLa cell assay, resulting in a very high selectivity index of 37,000 (Table 3). Although ISG-21 displays good efficacy and selectivity in vitro, analysis of its in vitro Absorption, Distribution, Metabolism, and Elimination (ADME) properties revealed a number of notable limitations that could affect its efficacy as an effective anti-malarial treatment option. In an assay that tests the ability of compounds to pass through a Caco-2 cell monolayer, ISG-21 exhibited no permeability (Pₐₚₚ<<<0.5×10⁻⁶ cm/s), suggesting an incapacity for use as an oral agent (typically Pₐₚₚ=5×10⁻⁶ cm/s is considered necessary). Additionally, ISG-21 was shown to be highly bound by serum proteins, a problematic feature for drug development that limits available drug for efficacy and provides a risk for variable levels of exposure among diverse populations of patients that vary in serum protein composition. Although this hit compound displays good potency and selectivity, the important drawbacks referenced above in its in vitro ADME properties significantly hampers its developability as an antimalarial drug.

Therefore, there is currently an urgent need for the discovery and development of a safe and effective antimalarial drug. Advantageously, herein we describe the discovery that analogs of ISG-21 containing an appropriately substituted 6-membered heterocycle in place of the terminal aryl ring of IS G-21 overcomes the above-referenced negative effects by substantially improving Caco-2 permeability and lowering serum binding without sacrificing other promising features, allowing for the establishment of efficacy in animal models of infection. As such, the novel compounds described herein are ideal candidates for developing a safe and effective therapeutic for treating/preventing malaria in mammals.

TABLE 3
In vitro inhibitory properties of ISG-21 from high throughput screening of the PSAC
target.

| | | | | IC₅₀ | CC₅₀ | Selectivity |
|---|---|---|---|---|---|---|
| Entry | Compound | Structure | K_{0.5} (μM)ª | (μM)^{b} | (μM)^{c} | (CC₅₀/IC₅₀)^{d} |
| 1 | ISG-21 | 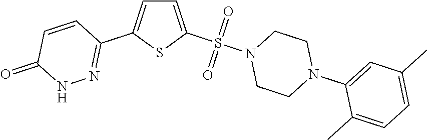 [SMILES: Cc1ccc(C)c(N2CCN(S(=O)(=O)c3ccc(-c4ccc(=O)[nH]n4)s3)CC2)c1] | 0.003 | 0.002 | 86 | 37,000 |

ªActivity in the PSAC assay;
^{b}Pf growth inhibition in PGIM;
^{c}Cytotoxicity against HeLa cells (3 days);
^{d}Selectivity index as calculated by dividing cytotoxicity (CC₅₀) by growth inhibition (IC₅₀).

TABLE 4
In vitro ADME properties of ISG-21 from high throughput screening of the PSAC target.

| | | | MLMS | MSS | Sol. | Caco-2 | Cyp 3A4 | |
|---|---|---|---|---|---|---|---|---|
| Entry | Compound | Structure | (% cons)ª | (% cons)^{b} | (μM)^{c} | Pₐₚₚ^{d} | % Inhib^{e} | PPB^{f} |
| 1 | ISG-21 | 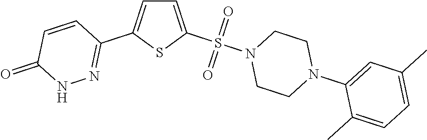 [SMILES: Cc1ccc(C)c(N2CCN(S(=O)(=O)c3ccc(-c4ccc(=O)[nH]n4)s3)CC2)c1] | 17 | 0 | 25 | 0.00 | 0 | 99.9 |

^{a}stability against murine liver microsomes, % consumed after 30 min.;
^{b}stability against mouse serum exposure, % consumed after 60 minutes;
^{c}solubility limit in H₂O
^{d}Caco-2 permeability, Pₐₚₚ × 10⁻⁶ cm/sec;
^{e}cyp 3A4 inhibition at 5 μM;
^{f}percent protein bound in mouse serum by equilibrium dialysis.

### SUMMARY OF THE INVENTION

Accordingly, the present invention is directed to the discovery of a novel class of anti-malarial pyridazinone compounds as represented by Formula I:

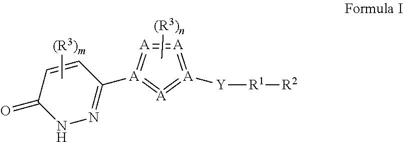

wherein:
A is independently selected from C, S, O or N combined through either single or double bonds to form a five-member heteroaromatic ring of 1-4 carbon atoms, 0-3 nitrogen atoms, 0-1 oxygen atom, and 0-1 sulfur atom;
R³ is a monovalent substituent group independently selected from alkenyl, alkoxy, alkyl, alkynal, having from 1 to 12 carbons, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halo, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent group is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, said substituent group may be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
n is an integer from 0-3;
m is an integer from 0-3;
Y is a divalent radical bridging A and R¹ selected from the group comprising, —COCH₂—, —SO₂—, —CO—, —CH₂—, —CH(CH₃)—, —NHCO—, NCH₃CO—, —CONH—, —CONCH₃—, —O(CO)—, —(CO)O—, —NH—, or —O—;
R¹ is a divalent non-aromatic, heterocyclic ring of 5-7 members containing 0-2 nitrogen atoms, 0-1 oxygen atom, and 3-6 carbon atoms, with the proviso that Y and R² are separated by at least 3 atoms, which non-aromatic, heterocyclic ring may bear 0-3 substituent groups defined as for R³, with the proviso that two or more such substituent groups on R¹ may be fused with R¹ to form one or more cycloalkyl, heterocyclic, aromatic, or heteroaromatic rings, or alternatively R¹ may be fused, optionally incorporating 0-2 substituent groups, with R² to form a fused heterocyclyl ring of 3-7 members, optionally substituted with 0-2 substituent groups defined as for R³;
R² is a 5- or 6-membered heteroaryl ring bearing 0-4 substituent groups independently selected from substituent groups defined as for R³, or substituents on R² may be optionally fused to R² to form one or more cycloalkyl, heterocyclic, aryl or heteroaryl rings, or 0 -2 R² substituents may, together with R¹, form a fused substituted or unsubstituted heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; or a pharmaceutically acceptable salt thereof.

In another embodiment, the present invention is directed to the discovery of a novel class of anti-malarial pyridazinone compounds as represented by Formula I(a):

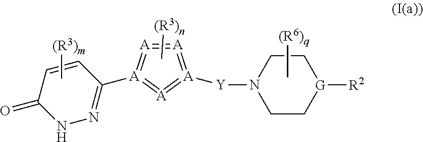

wherein:
G is selected from C or N and is part of a heterocyclic ring which is optionally substituted with (R⁶)_{q}, where q is an integer from 0-4; and
R⁶ is as defined for R³, with the additional proviso that R⁶ substituents on the heterocyclic ring containing G may be optionally fused to each other or a carbon atom of the ring containing G to form one or more cycloalkyl, heterocyclic, aromatic, or heteroaromatic rings; or 0-2 substituents on the heterocyclic ring containing G may, together with R², form a fused substituted or unsubstituted cycloalkyl or heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
R² is a 5- or 6-membered heteroaryl ring bearing 0-4 substituents independently selected from substituent groups defined as for R³, or substituents on R² may be optionally fused to R² to form one or more cycloalkyl, heterocyclic, aryl, or heteroaryl rings, of 3-8 members; or a pharmaceutically acceptable salt thereof.

In yet another embodiment, the present invention is directed to the discovery of a novel class of anti-malarial pyridazinone compounds as represented by Formula I(b):

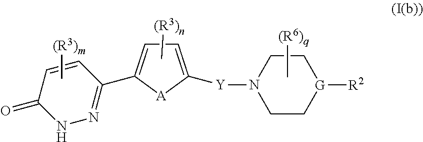

wherein:
A is independently selected from O, S or N, wherein,
n is an integer from 0-2 when A is O or S, and
n is an integer from 0-3 when A is N;
or a pharmaceutically acceptable salt thereof.

In another embodiment, compounds of the present invention are selected from:

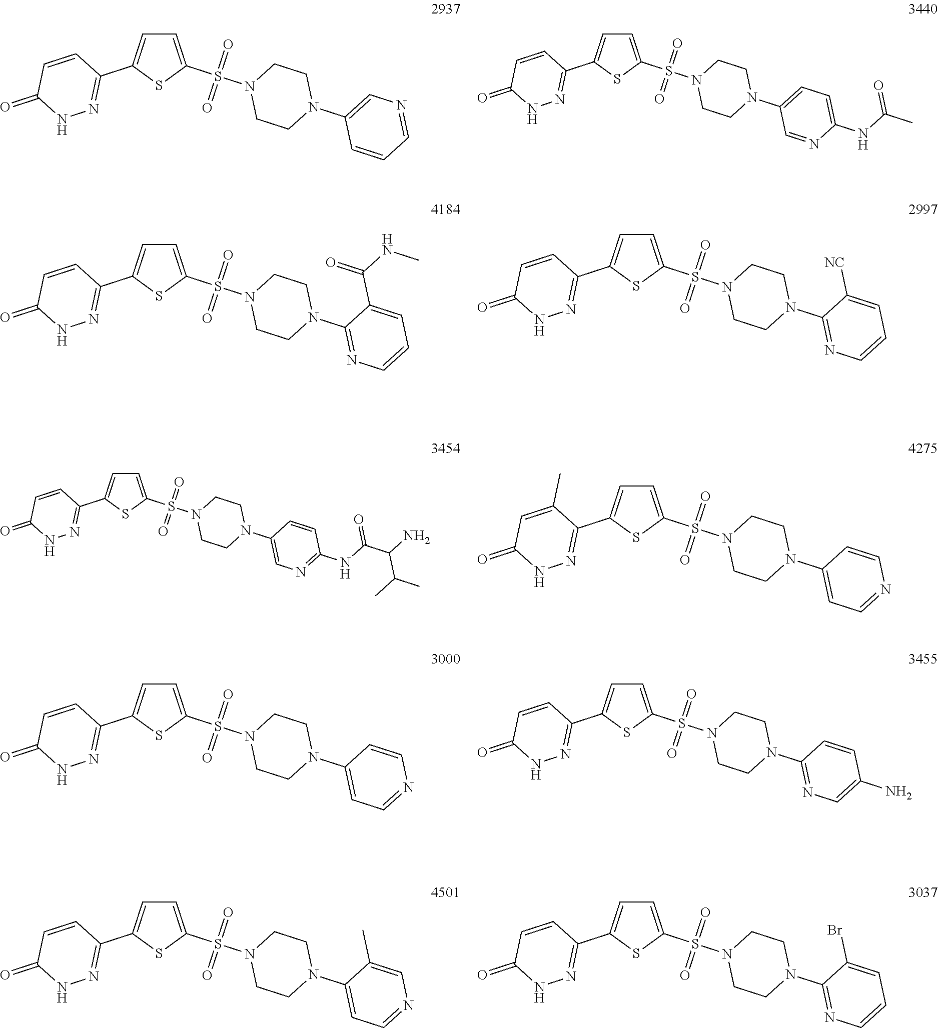

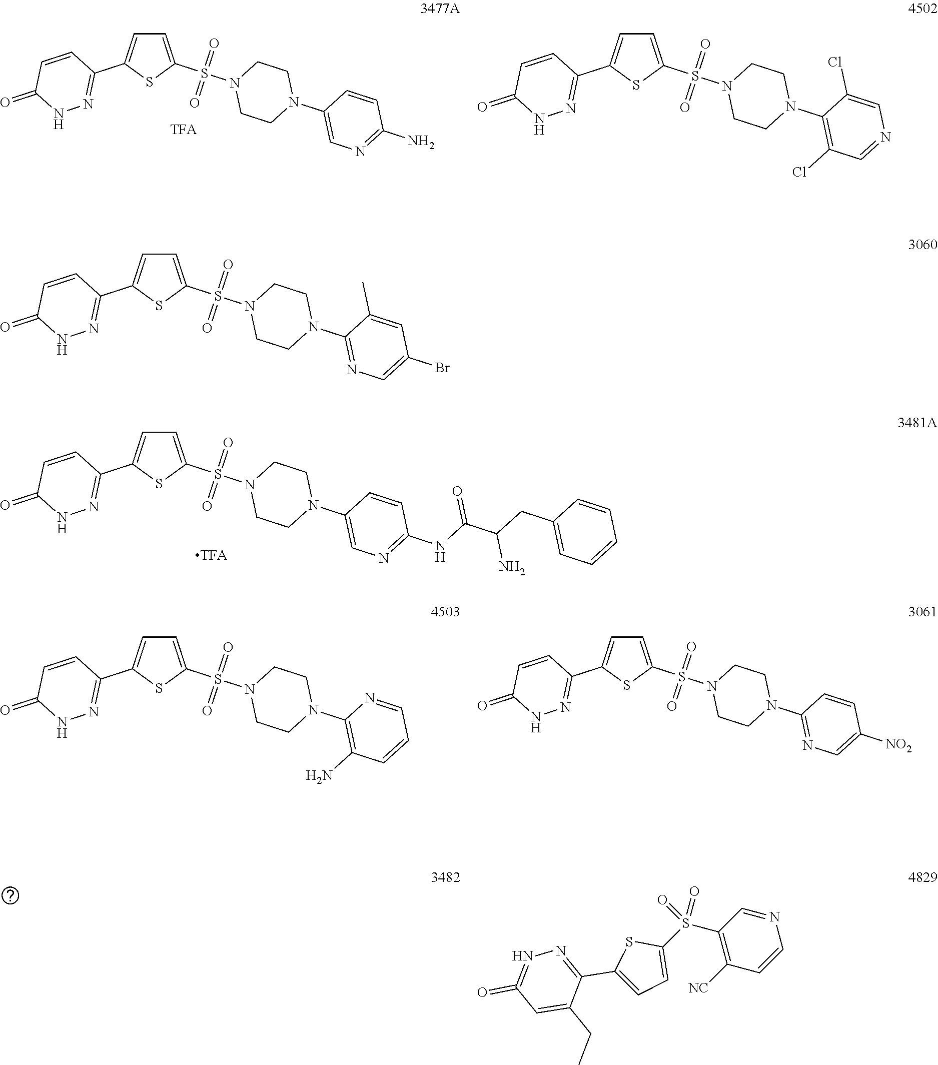

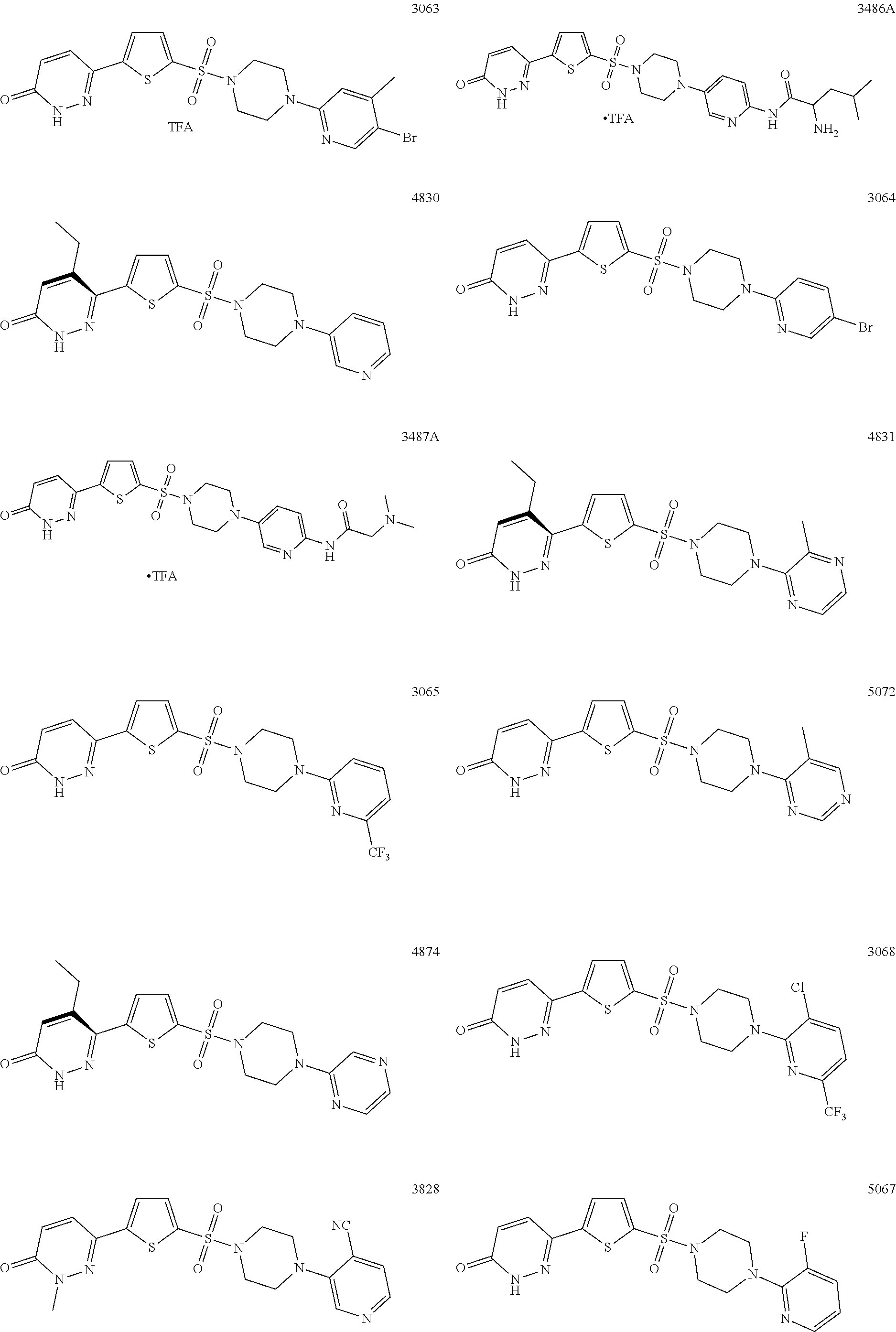

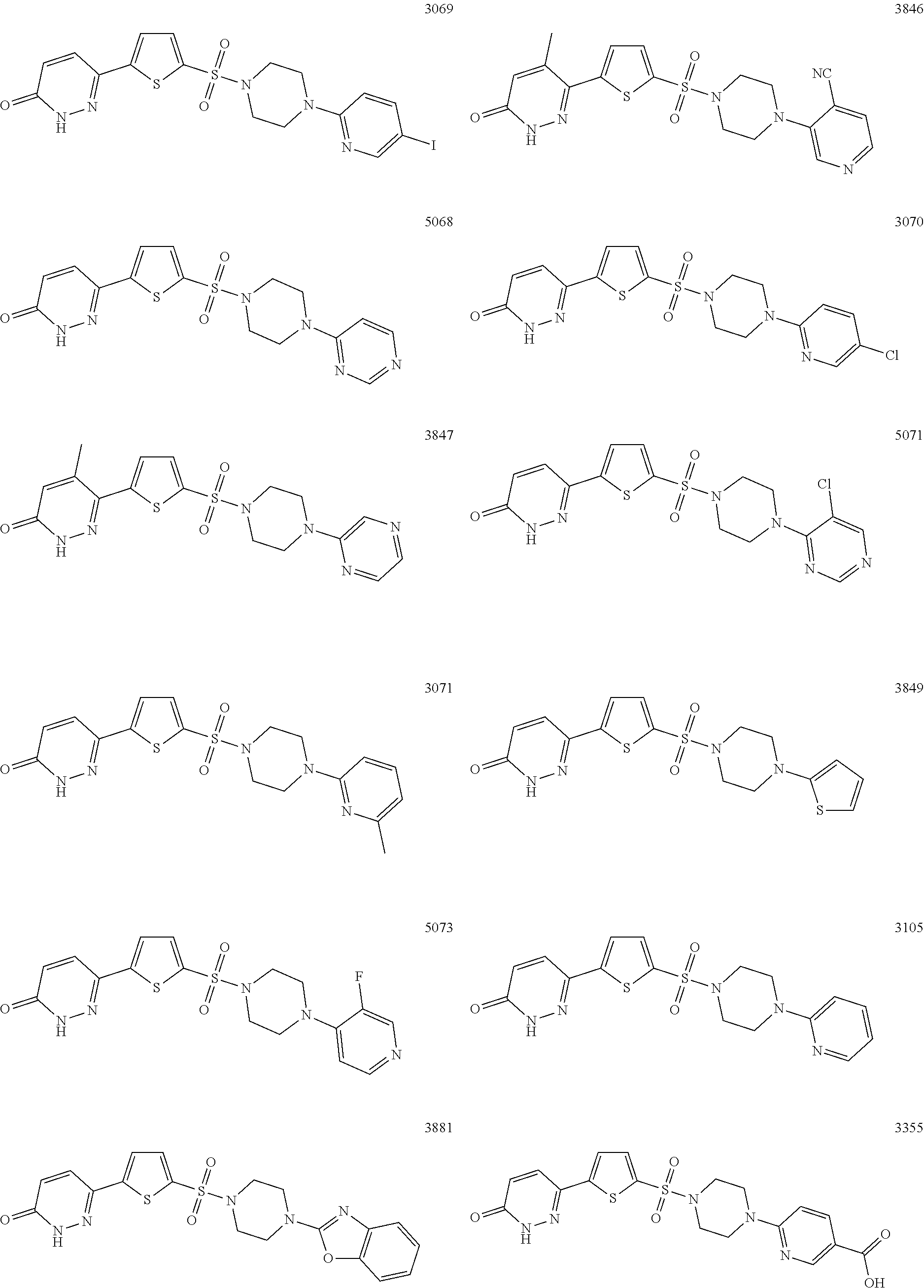

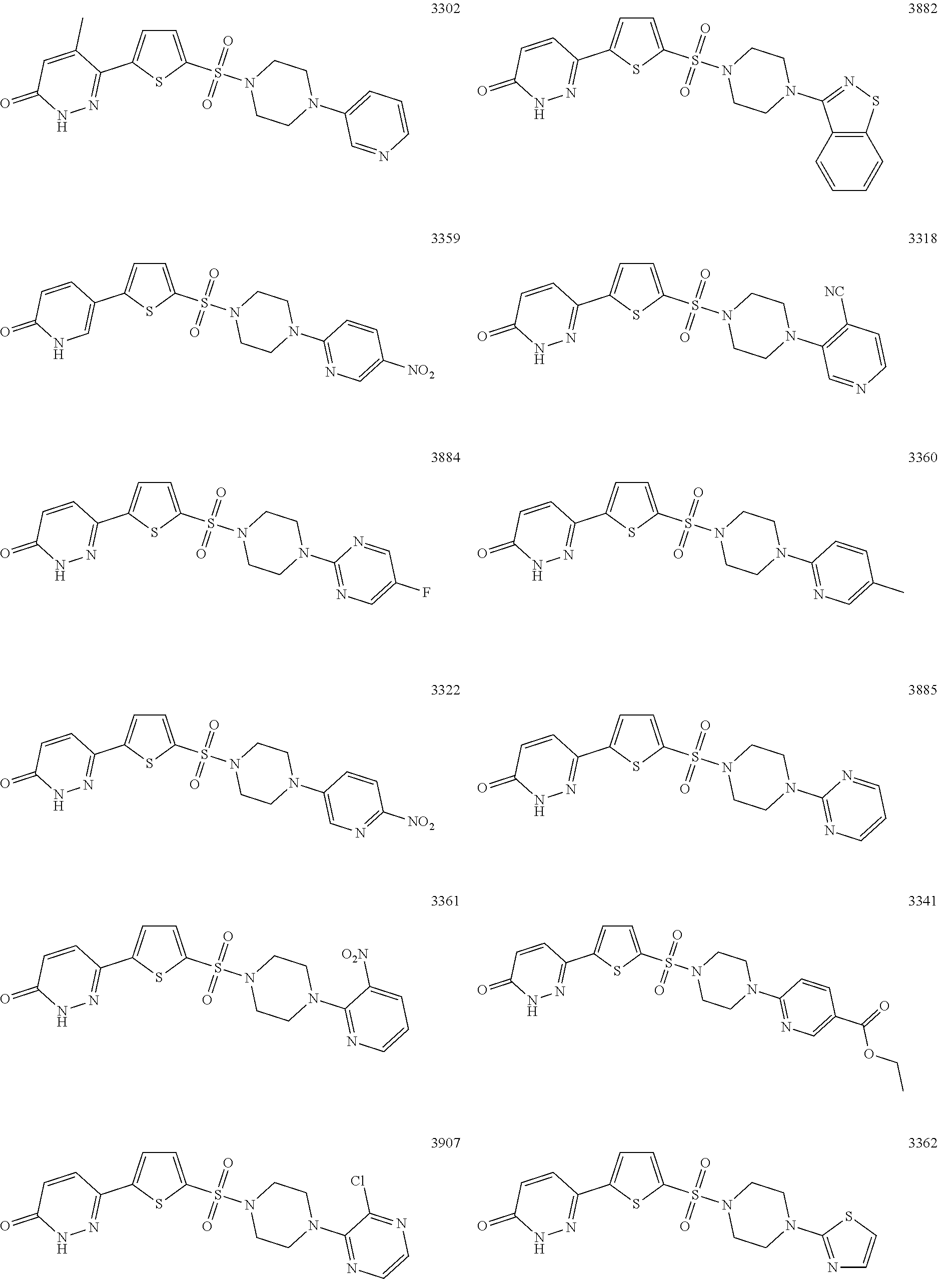

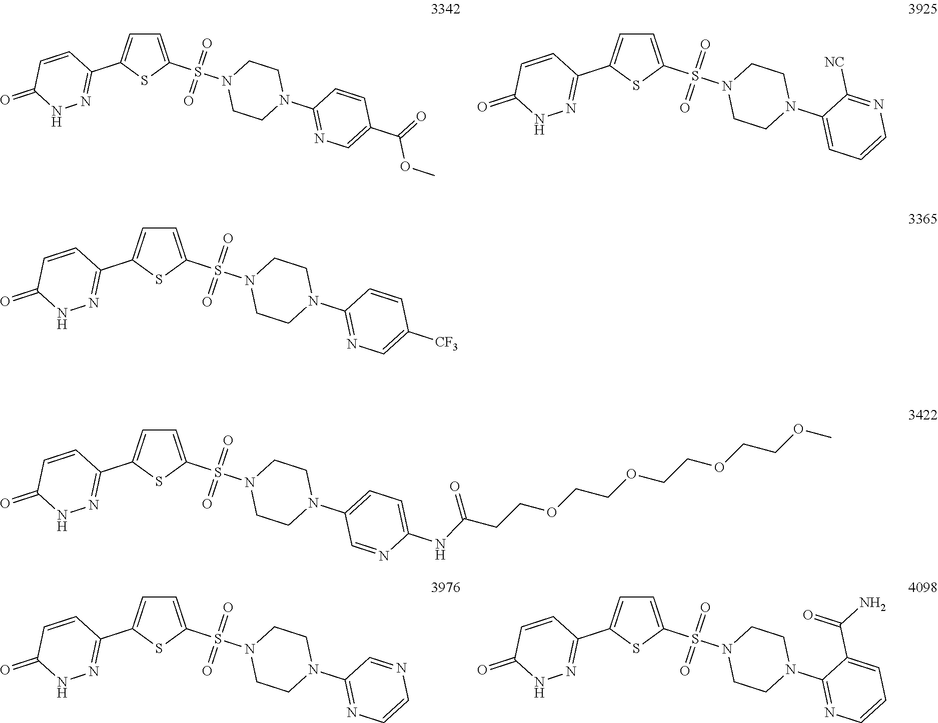 [SMILES: COC(=O)c1ccc(N2CCN(S(=O)(=O)c3ccc(-c4ccc(=O)[nH]n4)s3)CC2)nc1 ; N#Cc1ncccc1N1CCN(S(=O)(=O)c2ccc(-c3ccc(=O)[nH]n3)s2)CC1 ; O=c1ccc(-c2ccc(S(=O)(=O)N3CCN(c4ccc(C(F)(F)F)cn4)CC3)s2)n[nH]1 ; COCCOCCOCCOCCC(=O)Nc1ccc(N2CCN(S(=O)(=O)c3ccc(-c4ccc(=O)[nH]n4)s3)CC2)cn1 ; O=c1ccc(-c2ccc(S(=O)(=O)N3CCN(c4cnccn4)CC3)s2)n[nH]1 ; NC(=O)c1cccnc1N1CCN(S(=O)(=O)c2ccc(-c3ccc(=O)[nH]n3)s2)CC1]

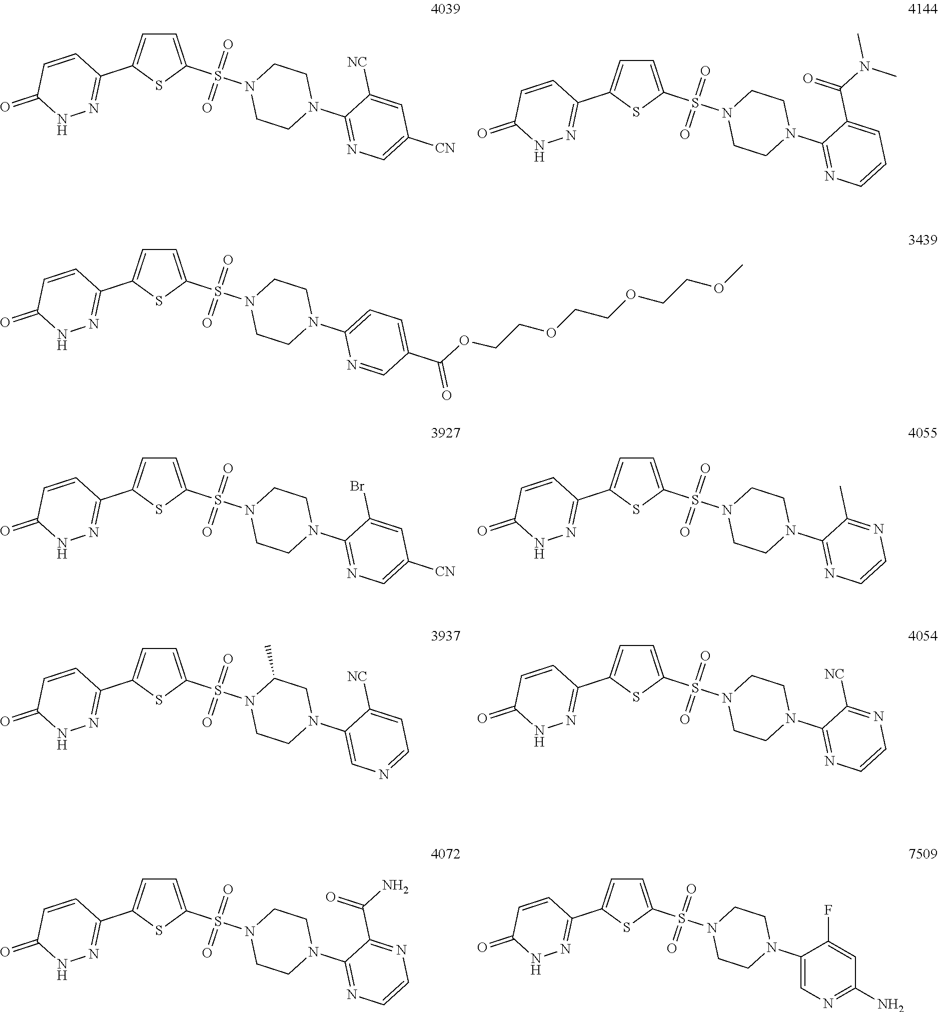

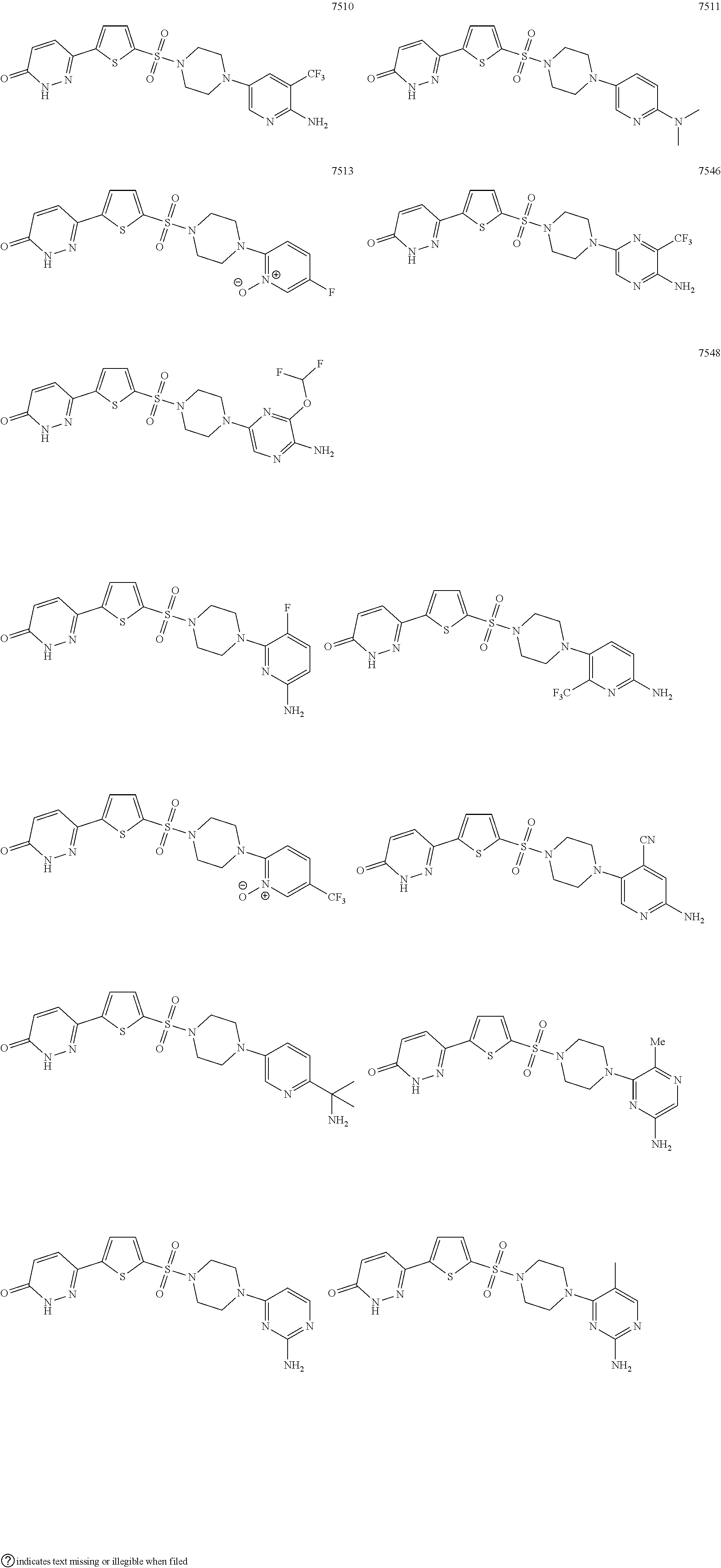

Representative compounds of Formula I, including Formulas I(a) and I(b), exhibit potent anti-malaria activity both in vitro and in vivo. Therefore, in one embodiment, the present invention is directed to pharmaceutical compositions comprising the anti-malaria compounds of Formula I, Formula I(a), and/or Formula I(b).

In another embodiment, the present invention is directed to a method of treating or preventing malaria in a mammalian subject comprising administering an effective amount of a pharmaceutical composition comprising the novel compounds of Formula I, Formula I(a), and/or Formula I(b) to a mammal in need thereof. In a preferred embodiment, the mammal is a human.

In another embodiment, the present invention is directed to the use of the compounds of Formula I, Formula I(a), and/or Formula I(b), in the manufacture of a medicament for treating or preventing malaria infection in a mammal. In a preferred embodiment, the mammal is a human.

In another embodiment, the anti-malaria compounds of the present invention as represented by Formulas I, I(a), and/or I(b) may be administered in combination with a second anti-malarial compound.

In a preferred embodiment, the present invention is directed to a method for treating or preventing malaria caused by a virus belonging to the genera Plasmodium comprising administering an effective amount of a pharmaceutical composition comprising the novel compounds of Formula I, Formula I(a), and/or Formula I(b), to a mammal in need thereof. Particular species of Plasmodium known to cause malaria treatable according to the present invention include, P falciparum, P. vivax, P. ovale, and P. malariae. In a preferred embodiment, the present invention is directed to a method for treating or preventing malaria caused by P. falciparum.

Multiple routes of administration are conceivable for compositions comprising Formula I, Formula I(a), and/or Formula I(b), and highly cost-effective production strategies can be easily achieved.

In another embodiment, the anti-malaria compounds of the present invention are formulated into a pharmaceutically-acceptable carrier or excipient and may be administered by injection, including, without limitation, intradermal, transdermal, intramuscular, intraperitoneal and intravenous. According to another embodiment of the invention, the administration of the anti-malaria compounds may be by oral administration and may be presented, for example, in the form of a tablet or encased in a gelatin capsule or a microcapsule, which simplifies oral application. The production of these forms of administration is within the general knowledge of a technical expert.

In another embodiment, the present invention is directed to a pharmaceutical composition for treating or preventing malaria in a mammalian subject comprising a compound of Formula II:

Q-Y—R¹—R² (II)
wherein:
Q is a heteroaryl ring of 5 members having group Y bound to the ring at a non-adjacent site to a 6-pyridazin-3-(2H)-one, 5-pyridin-2(1H)-one, a substituted carboxamide, or a substituted carboxylate moiety, wherein Q is optionally substituted on either the heteroaryl ring, the 6-pyridazin-3-(2H)-one moiety, or both, with one or more substituent groups independently selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, and thiocarbonyl, each substituent group may be optionally substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, and thiol;
Y is a divalent radical bridging Q and R¹ selected from the group comprising: —COCH₂—, —CH₂CO—, —SO₂—, —CO—, —CH₂—, —CH(CH₃)—, —NHCO—, —NCH₃CO—, —CONH—, —CONCH₃—, —O(CO)—, —(CO)O—, —NH—, and —O—;
R¹ is a divalent non-aromatic heterocyclic ring of between 5-7 members containing 0-2 nitrogen atoms, 0-1 oxygen atoms, and 3-6 carbon atoms, with the proviso that Y and R² are separated by at least 3 atoms, which non-aromatic, heterocyclic ring may bear 0-3 substituent groups selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when the substituent group is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, each substituent can be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol, with the proviso that two or more such substituent groups on R¹ may be fused with R¹ to form one or more cycloalkyl or heterocyclic rings, or alternatively R¹ may be fused with R² to form a fused cycloalkyl or heterocyclyl ring of 3-7 members, optionally substituted with 0-2 substituent groups selected from alkenoxy, alkenyl, alkoxy, alkyl, alkylamino, alkynal, alkynoxy, amide, amidino, amino, aminoalkyl, aminoaryl, aryl, arylalkyl, aryloxy, azido, azo, carbamate, carbamide, carbonyl, carboxamide, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; and
R² is a 5- or 6-membered heteroaryl ring bearing 0-4 substituent groups independently selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, said substituent group may be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; or
substituents on R² may be optionally fused to R² to form one or more cycloalkyl, heterocyclic, aryl or heteroaryl rings; or 0-2 R² substituents may, together with R¹, form a fused substituted or unsubstituted cycloalkyl or heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
or a pharmaceutically acceptable salt thereof.

Compounds according to Formula II include the following:

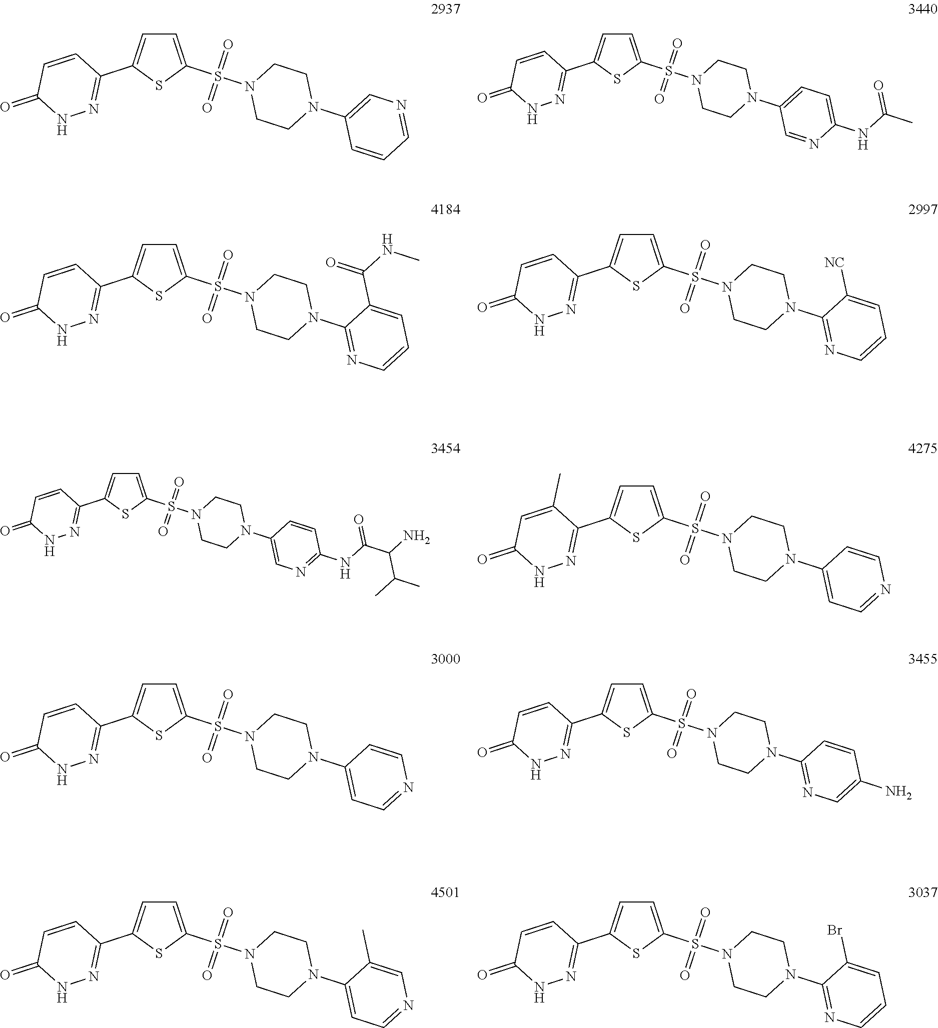

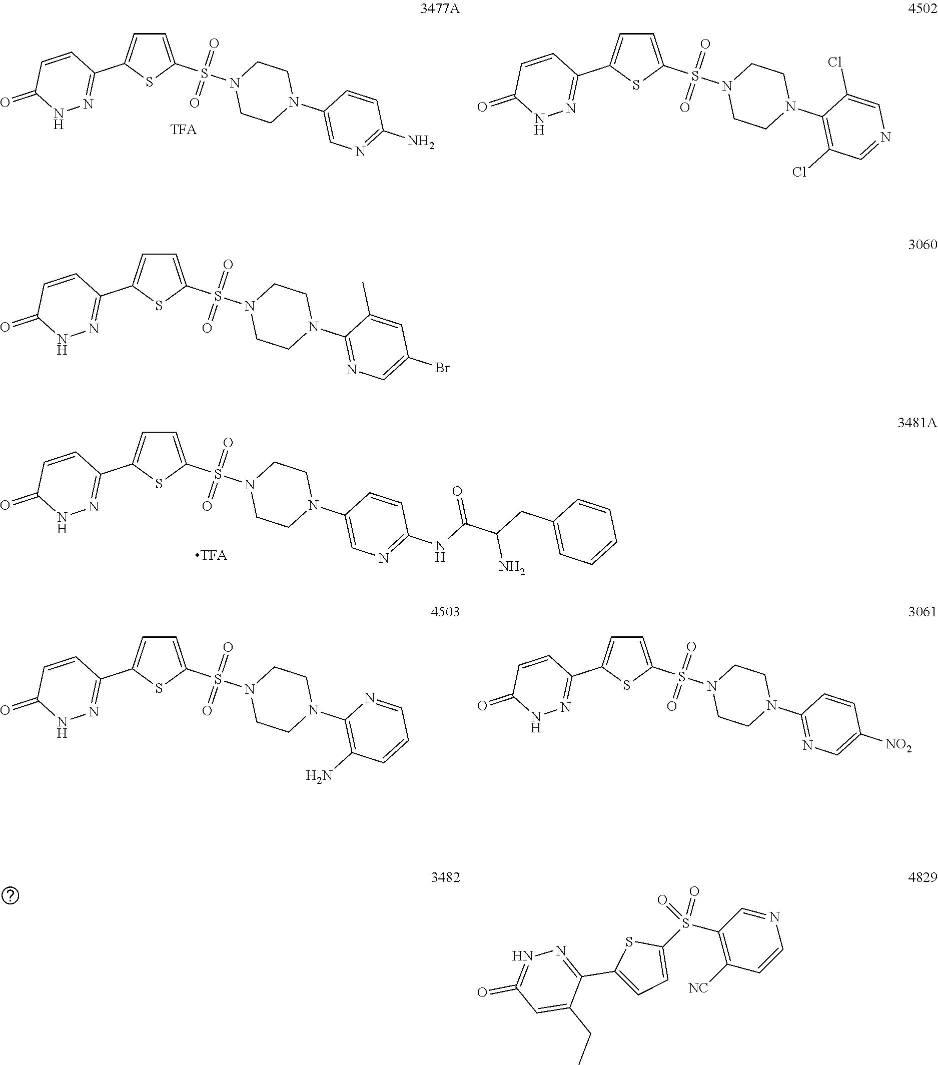

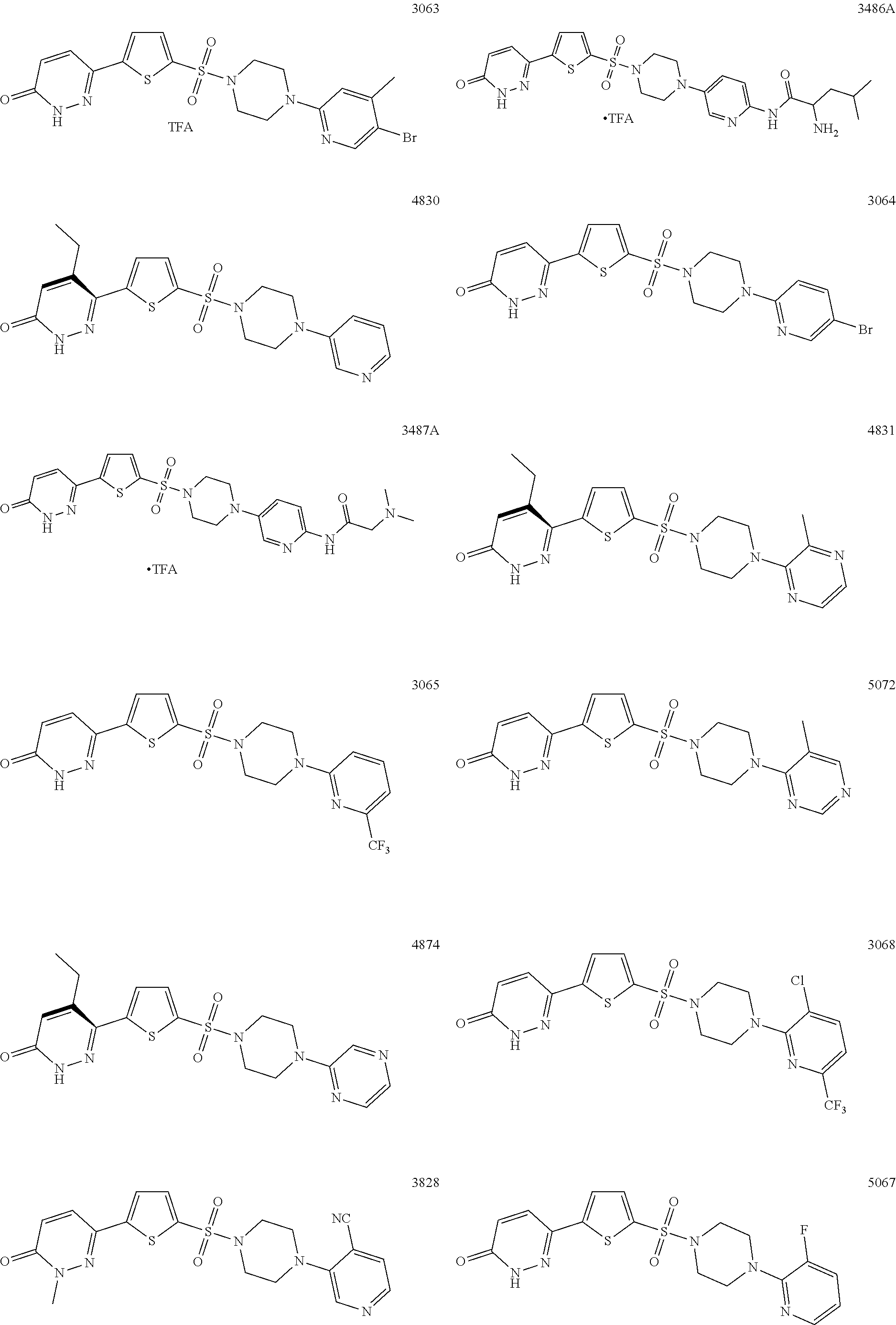

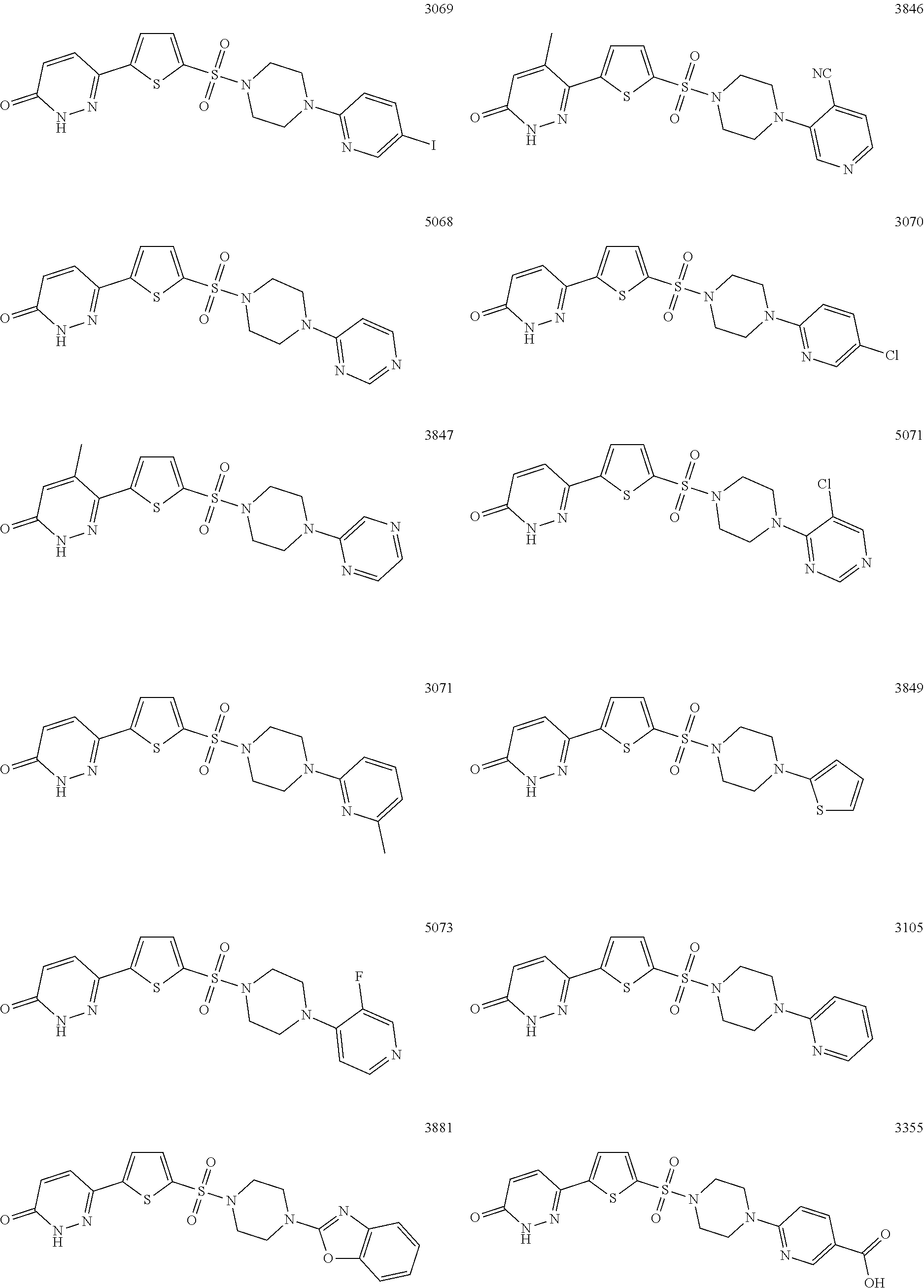

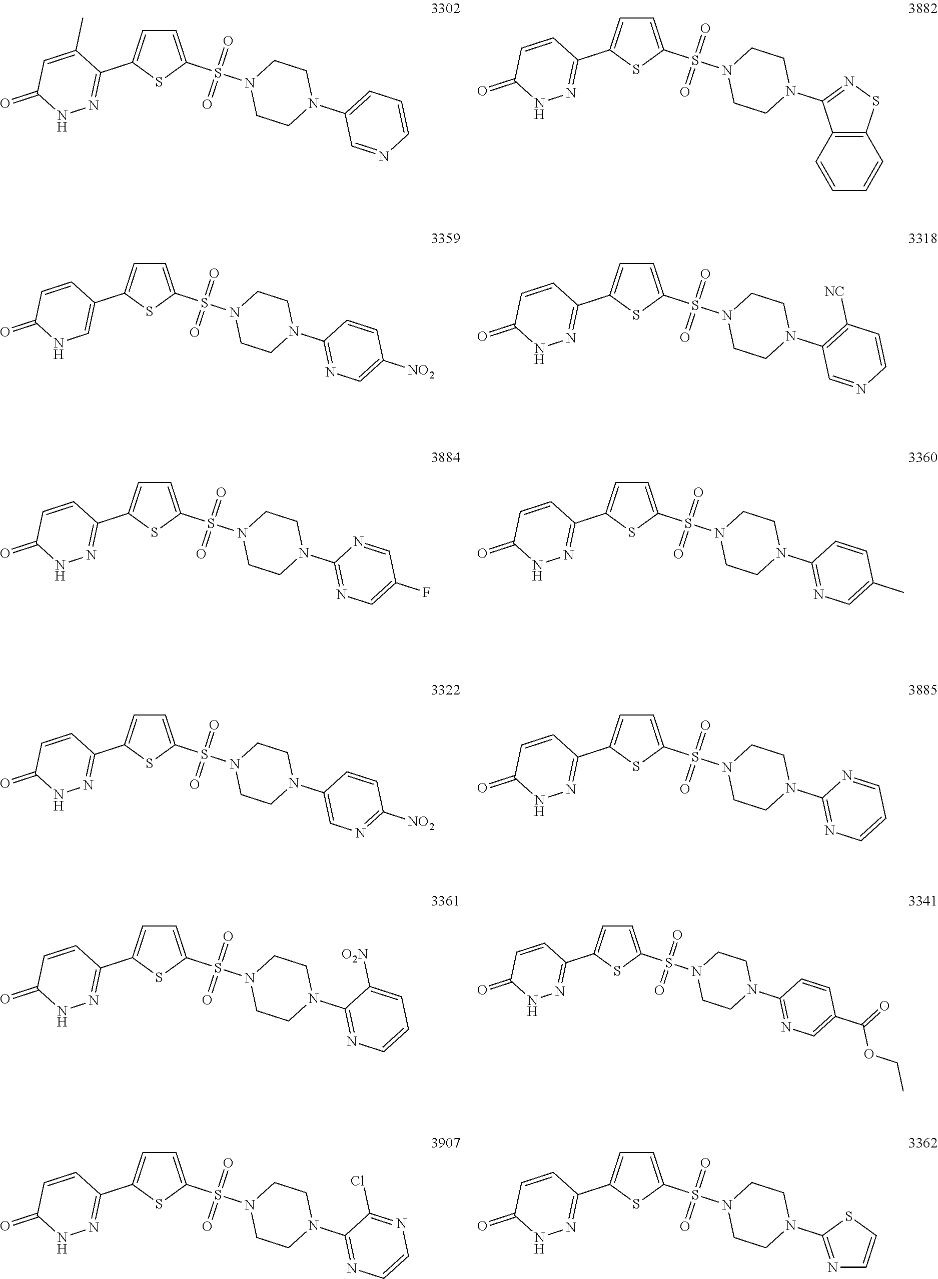

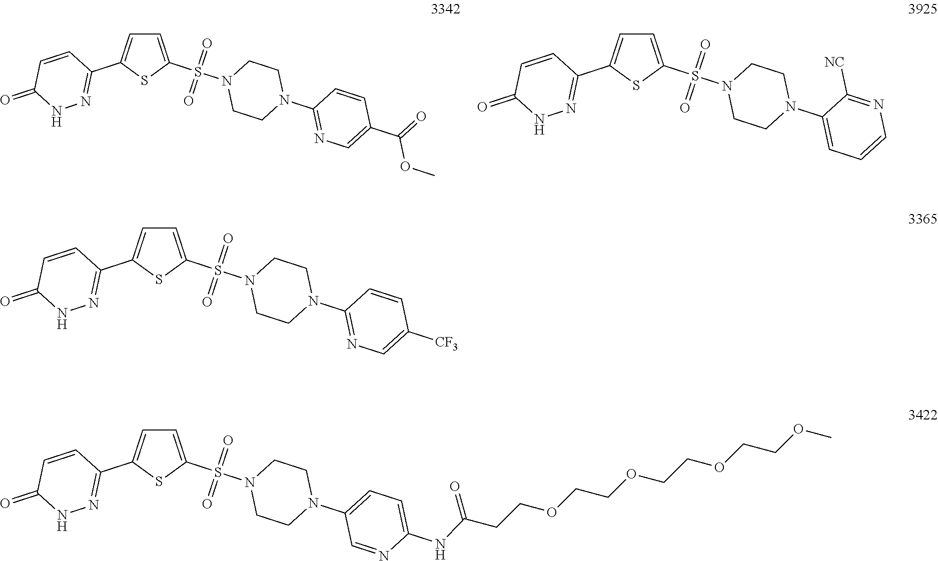 [SMILES: COC(=O)c1ccc(N2CCN(S(=O)(=O)c3ccc(-c4ccc(=O)[nH]n4)s3)CC2)nc1 ; N#Cc1ncccc1N1CCN(S(=O)(=O)c2ccc(-c3ccc(=O)[nH]n3)s2)CC1 ; O=c1ccc(-c2ccc(S(=O)(=O)N3CCN(c4ccc(C(F)(F)F)cn4)CC3)s2)n[nH]1 ; COCCOCCOCCOCCC(=O)Nc1ccc(N2CCN(S(=O)(=O)c3ccc(-c4ccc(=O)[nH]n4)s3)CC2)cn1]

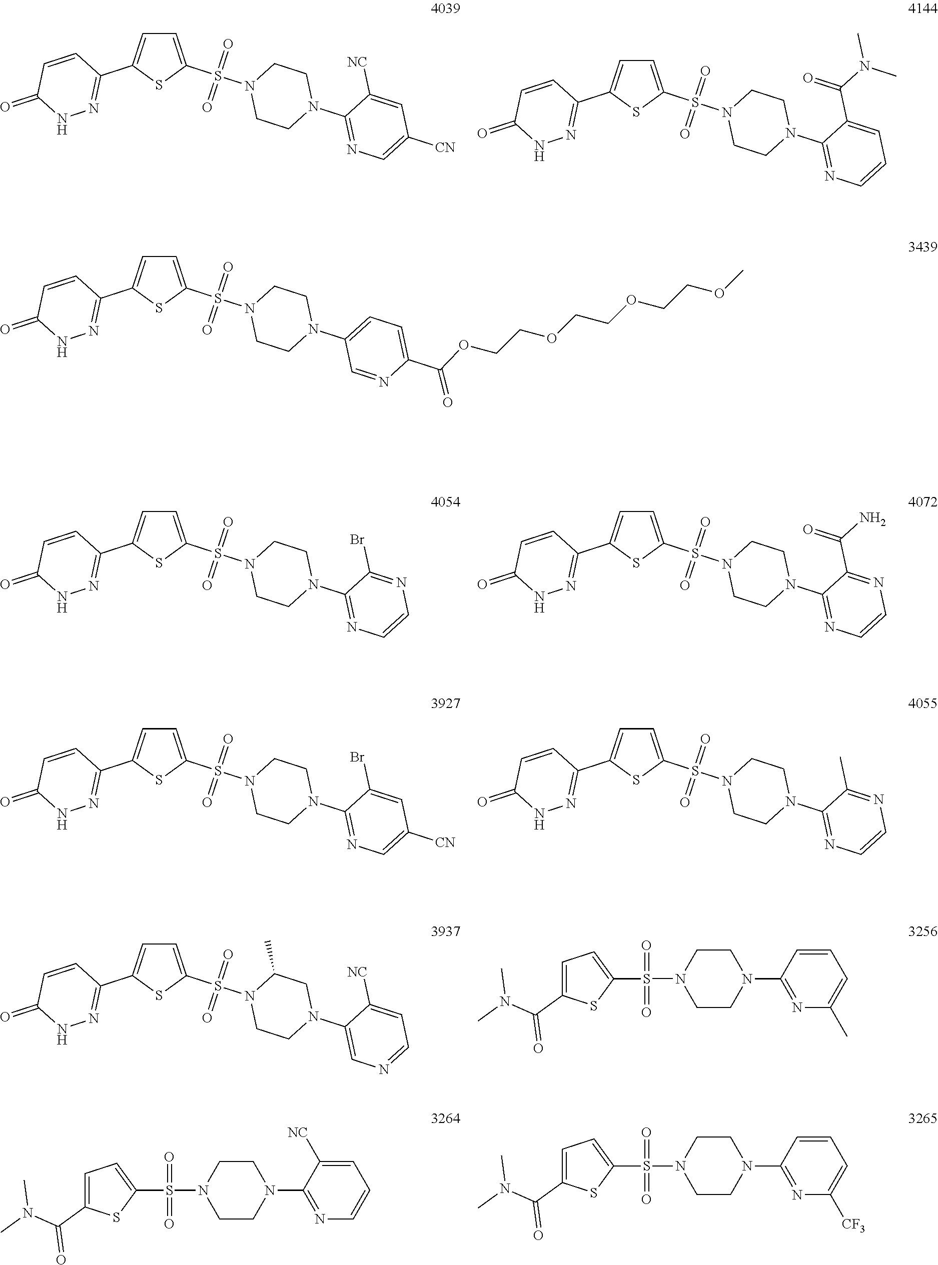

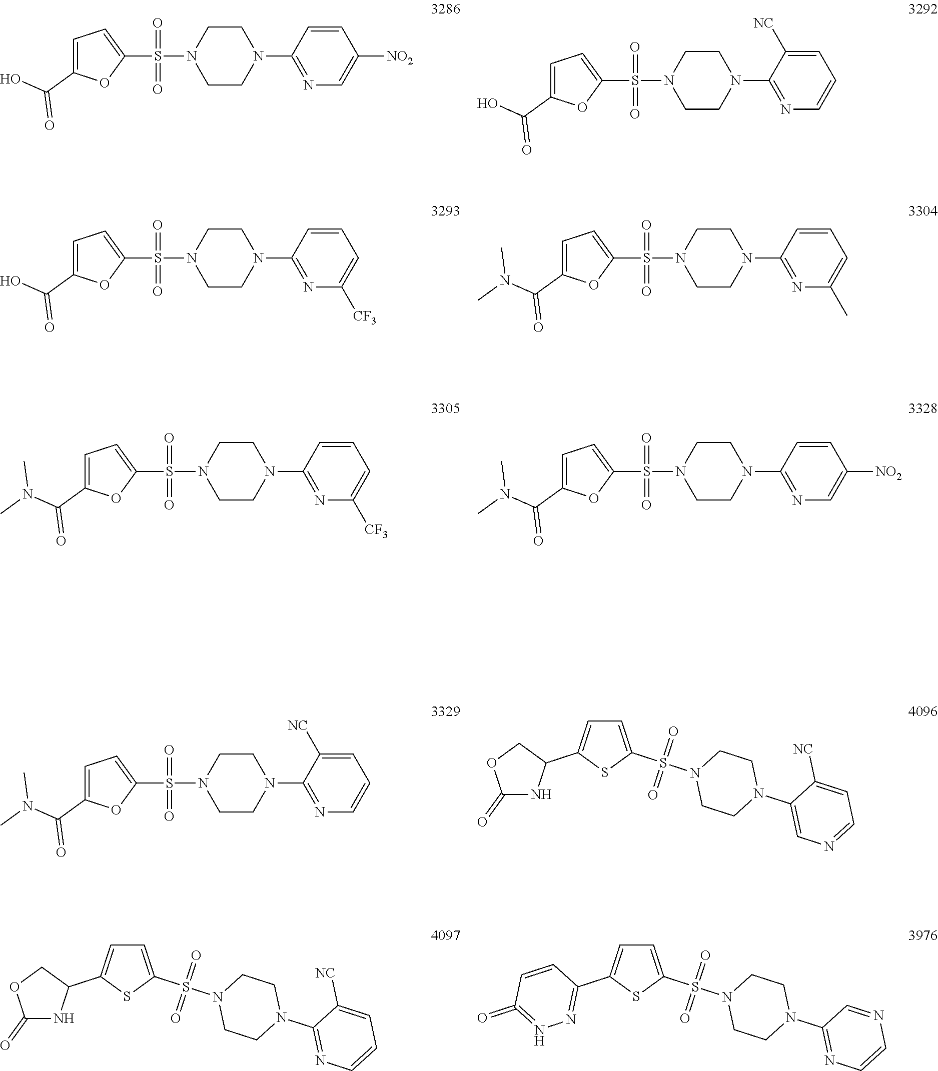

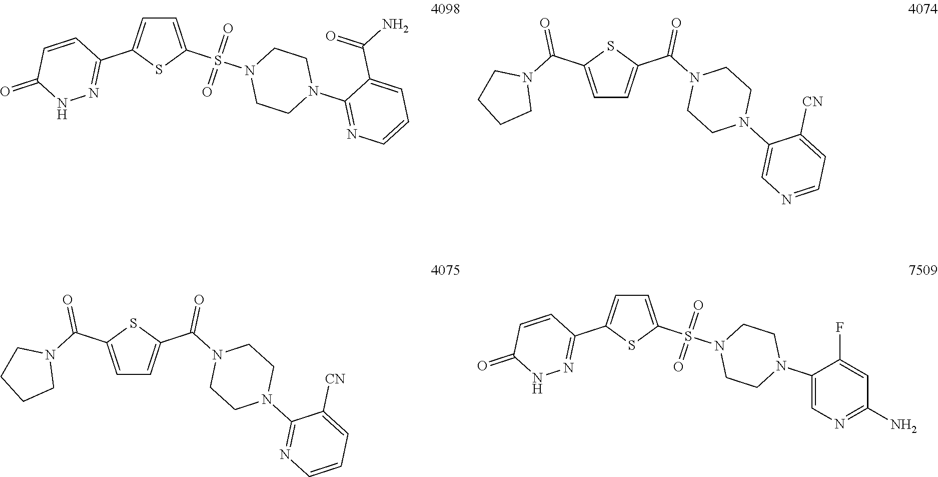 [SMILES: NC(=O)c1cccnc1N1CCN(S(=O)(=O)c2ccc(-c3ccc(=O)[nH]n3)s2)CC1 ; N#Cc1ccncc1N1CCN(C(=O)c2ccc(C(=O)N3CCCC3)s2)CC1 ; N#Cc1cccnc1N1CCN(C(=O)c2ccc(C(=O)N3CCCC3)s2)CC1 ; Nc1cc(F)c(N2CCN(S(=O)(=O)c3ccc(-c4ccc(=O)[nH]n4)s3)CC2)cn1]

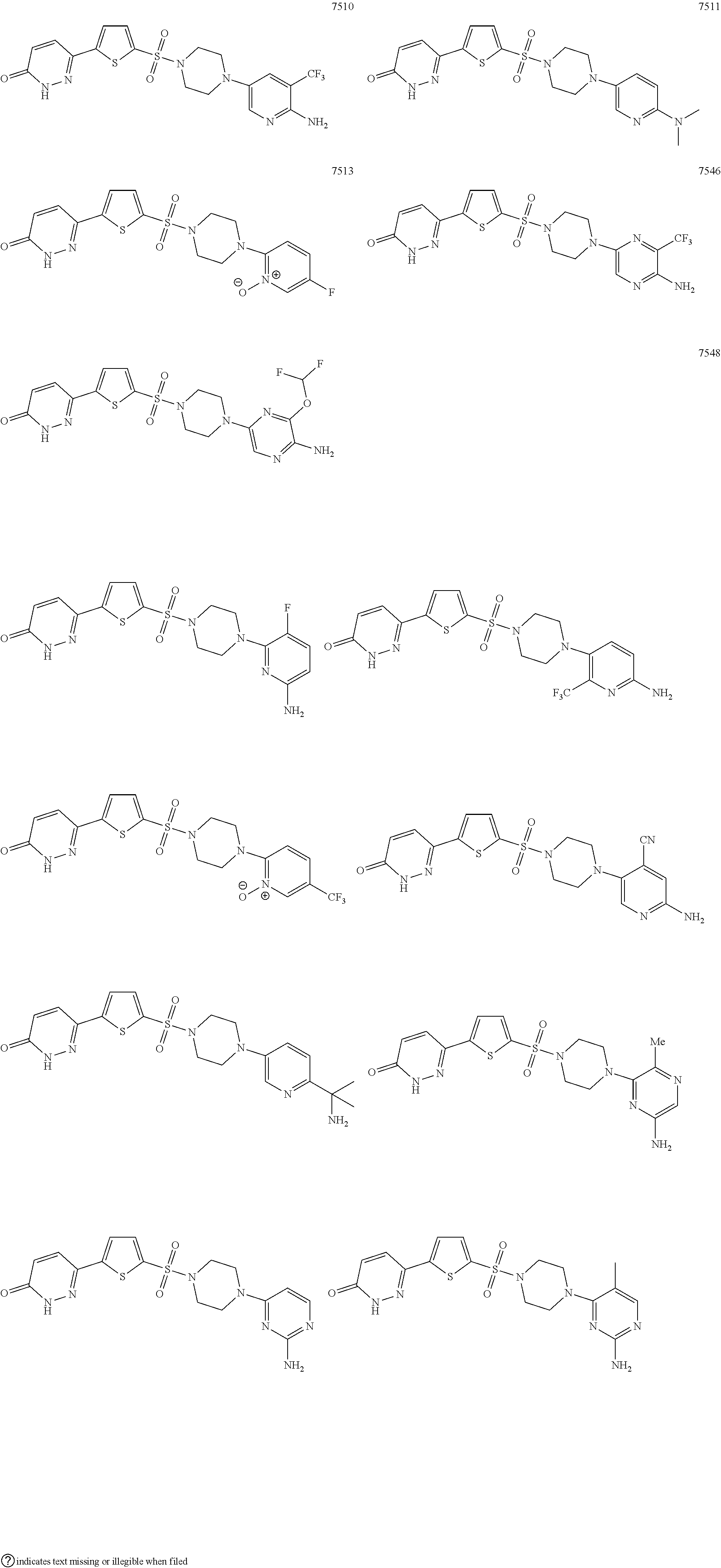

In another embodiment, the present invention is directed to a method for treating or preventing malaria in a mammalian subject comprising administering to said subject an effective amount of a pharmaceutical composition comprising compounds of Formula II. In a preferred embodiment, the mammal is a human.

In another embodiment, the pharmaceutical composition comprising compounds of Formula II may be administered in combination with a second antimalarial agent.

In another embodiment, the present invention is directed to the use of a pharmaceutical composition comprising Formula II for treating or preventing malaria infection in a mammalian subject. In a preferred embodiment, the mammal is a human.

In another embodiment, the present invention is directed to the use of a pharmaceutical composition comprising Formula II in the manufacture of a medicament for treating or preventing malaria infection in a mammalian subject. In a preferred embodiment, the mammal is a human.

### BRIEF DESCRIPTION OF THE DRAWINGS

FIG. 1 shows the clearance rates (PK) for pyridazinone compounds MBX-3318, MBX-3976, and MBX-4055 following IV and PO administration of the compounds to CD-1 mice. The results show favorable half-lives for all three compounds.

FIG. 2 shows the results of administration of MBX-3318 in a humanized mouse model following infection with P. falciparum. FIG. 2A shows MBX-3318 reduced parasite growth by over 70% in infected mice as compared to control mice. FIG. 2B shows the clearance rate of MBX-3318 over time in the infected mice. The IC₅₀ levels demonstrate the compound remains in the system for a sufficient time to be effective against growth of the parasite.

(M1=mouse 1; M2=mouse 2; ED90=90% Effective dose; 3D7=Plasmodium falciparum malaria parasite 3D7 (from the NF54 strain; sensitive to all antimalarials); LQ=Limit of quantitation).

FIG. 3 shows the results of administration of pyridazinone compound MBX-4055 in a humanized mouse model following infection with P. falciparum. FIG. 3A shows 50 mg/kg MBX-4055 administered once daily reduced parasite growth by over 90% in infected mice as compared to control mice. FIG. 3B shows the clearance rate of MBX-4055 over time in the infected mice. The IC₅₀ levels demonstrate the compound remains in the system for a sufficient time to be effective against growth of the parasite.

### DEFINITIONS

The term “alkyl” refers to a linear or branched saturated hydrocarbon group of from 1 to 12 carbons in total. Alkyl includes substituted and unsubstituted alkyl groups. Examples of alkyl groups include methyl, ethyl, n-propyl, isopropyl, n-butyl, isobutyl, t-butyl, n-pentyl, isopentyl, neopentyl, n-hexyl, n-heptyl, n-octyl.

The term “alkenyl” refers to an unsaturated hydrocarbon group of from 2 to 12 carbon atoms inclusive that possesses one or more carbon-carbon double bonds. Alkenyl includes substituted and unsubstituted alkenyl groups. Examples of alkenyl groups include vinyl, allyl, 1-propenyl, isopropenyl, 2-butenyl, 2-pentenyl and 2-hexenyl.

The term “alkenoxy” refers to an alkenyl group as defined above, connected to the parent molecular group through a divalent oxygen atom.

The term “alkoxy” refers to an alkyl, cycloalkyl, heterocyclyl, alkenyl, or an alkynyl group of 1 to 12 carbon atoms inclusive bonded to the parent molecular group through an etheric oxygen atom. Alkoxy includes substituted and unsubstituted alkoxy groups. Examples of alkoxy groups include methoxy, ethoxy, vinyloxy, allyloxy, butenoxy.

The term “alkylamino” refers to an alkyl group as defined above having one or more amino or aminoalkyl substituents.

The term “alkynyl” refers to an unsaturated hydrocarbon group of 2 to 12 carbon atoms in total that possesses one or more carbon-carbon triple bonds. Alkynyl includes substituted and unsubstituted alkynyl groups. Examples of alkynyl groups include ethynyl, propynyl, butynyl, pentynyl, hexynyl, heptynyl.

The term “alkynoxy” refers to an alkynyl group as defined above connected to the parent molecular group through a divalent oxygen atom.

The term “amide” refers to a group with the formula —C(O)N— in which the group is bound to 1-3 carbon containing groups through one single bond from the trivalent carbon and two single bonds to the nitrogen of the group. An amide group may be bound to the parent molecular group through either the trivalent carbon or the nitrogen of the group. The term amide includes groups with unsubstituted nitrogen atoms or primary amides, monosubstituted nitrogens or secondary amides, and disubstituted nitrogens or tertiary amides Amide includes substituted and unsubstituted amide groups.

The terms “amidino” or “amido” refer to a trivalent carbon atom bound to two nitrogen atoms and one carbon atom. The carbon atom of the amidino is either a component of the parent molecular group or a component of a substituent selected from alkyl, alkenyl, alkynyl, cycloalkyl, heterocyclyl, aryl, or heteroaryl. The nitrogen atoms of the group are optionally substituted with 0-3 groups independently selected from alkyl, alkenyl, alkynyl, cycloalkyl, heterocyclyl, aryl, or heteroaryl. The amidino is optionally attached to the parent molecular group through a single bond to one of the two nitrogen atoms. Amidinos include substituted and unsubstituted amidinos.

The term “amino” refers to the group —NH₂.

The term “aminoalkyl” refers to a nitrogen atom bonded to the parent molecular group and additionally bearing 1-3 groups independently selected from alkyl, cycicoalkyl, heterocyclyl, alkenyl, alkynyl, aryl, or heteroaryl. Aminoalkyl includes substituted and unsubstituted aminoalkyl groups Aminoalkyl includes ionic groups such as ammonium salts and tetraalkylammonium salts. Examples of aminoalkyl groups include methylamino, ethylamino, isopropylamine, dimethylamino

The term “aminoaryl” refers to a nitrogen atom bonded to 1-2 groups independently selected from aryl, or heteroaryl. Heteroaryl includes substituted and unsubstituted heteroaryl groups. Examples include phenylamino, diphenylamino, napthylamino, 2-pyridylamino.

The term “aryl” or “aromatic” refer to a planar, mono- or polycyclic moiety with a continuous system of pi-conjugated carbon atoms. Aryl includes both monocyclic and fused polycyclic moieties, ranging in size from 5 to 14 carbon atoms, including 5- and 6-membered hydrocarbon and heterocyclic aromatic groups, as well as substituted and unsubstituted aryl groups. Examples of aryl groups include benzene, naphthalene, anthracenes, phenanthrene.

The term “arylalkyl” refers to an aryl group as defined above having at least one alkyl substituent.

The term “aryloxy” refers to an aromatic or heteroaromatic group bonded to the parent molecular group through an etheric oxygen atom. Aryloxy includes substituted and unsubstituted aryloxy groups. Examples of aryloxy groups include phenoxy, napthyloxy, 4-pyridyloxy and 2-furanyloxy.

The term “azido” refers to the group —N₃.

The term “azo” refers to the group —N═N— where the azo group is bonded to the parent molecular group and a group selected from alkyl, alkenyl, alkynal, cycloalkyl, heterocyclyl, aryl, or heteroaryl.

The term “carbamate” refers to a trivalent carbon atom with one double bond to an oxygen atom, one single bond to an oxygen atom and one single bond to a nitrogen atom. Carbamates attach to the parent molecular group through a single bond to either the nitrogen or the divalent oxygen. In addition to the parent molecular group, carbamates are directly substituted with 1-2 groups independently selected from alkyl, alkenyl, alkynyl, cycloalkyl, heterocyclyl, aryl, or heteroaryl. Carbamate includes substituted and unsubstituted carbamate groups.

The terms “carbamide” or “urea” refer to a trivalent carbon atom with one double bond to oxygen and two single bonds to nitrogens. Carbamides attach to the parent molecular group through a single bond to nitrogen. In addition to the parent molecular group, carbamides are directly substituted with 1-3 groups independently selected from alkyl, alkenyl, alkynyl, cycloalkyl, heterocyclyl, aryl, or heteroaryl. Carbamide includes substituted and unsubstituted carbamide groups.

The term “carbonyl” refers to a trivalent carbon atom possessing a double bond to oxygen and two single bonds to other elements in which one element is a component of the parent molecular group that the carbonyl is a substituent of and the other is selected from carbon, oxygen, nitrogen, sulfur, or hydrogen. Carbonyl groups include substituted and unsubstituted carbonyl groups. Examples of carbonyl groups include ketones, aldehydes, esters, amides, thioesters, carbamates, and carboxylic acids.

The term “carboxamide” refers to a group with the formula —C(O)N— in which the group is bound to the parent molecular group that the amide is a substituent of through a single bond from the trivalent carbon. The term carboxamide includes groups with unsubstituted nitrogen atoms or primary amides, monosubstituted nitrogens or secondary amides, and disubstituted nitrogens or tertiary amides.

The term “carboxylate” refers to a group with the formula —C(O)O— in which the group is bound to the parent molecular group through a single bond from the trivalent carbon. The term “carboxylate” includes both unsubstituted anionic compounds, hydrogen substituted carboxylic acids, and carbon substituted groups.

The term “cyano” refers to the group —CN.

The term “cycloalkyl” refers to a cyclic moiety composed of carbon and hydrogen. The term includes both monocyclic and fused polycyclic moieties ranging in size from 3 to 14 carbon atoms. Cycloalkyl includes substituted or unsubstituted cycloalkyl groups. Examples of cycloalkyl groups include cyclopropyl, cyclobutyl, cyclopentyl, cyclohexyl, cycloheptyl, cyclooctyl, cyclononane, cyclodecane, decalin.

The terms “ester” refers to a group with the formula —C(O)O— in which the group is bound to two carbon groups through single bonds from the trivalent carbon and the divalent oxygen of the group. An ester group may be bound to the parent molecular group through either the trivalent carbon or the divalent oxygen of the group.

The term “ether” refers to an alkyl, cycloalkyl, heterocyclyl, alkenyl, or an alkynyl group bonded through a —C—O—C— linkage to another alkyl, cycloalkyl, heterocyclyl, alkenyl, or an alkynyl group. Ether includes substituted and unsubstituted ether groups.

The term “fused” refers to a polycyclic ring system in which one ring contains one or more atoms, preferably 1-3 atoms, in common with one or more other rings.

The term “guanidine” refers to a trivalent carbon atom bound to three nitrogen atoms. Guanidines attach to the parent molecular group through a single bond to nitrogen. In addition to the parent molecular group, guanidines are directly substituted with 0-4 groups independently selected from alkyl, alkenyl, alkynyl, cycloalkyl, heterocyclyl, aryl, or heteroaryl. Guanidines include substituted and unsubstituted guanidine groups.

The term “haloalkoxy” refers to an alkoxy group having at least on halogen group.

The term “haloalkyl” refers to an alkyl group as defined above having at least one halogen substituent.

The term “halogen” refers to the group -X, where X is an element of group VIIA in the periodic table. Examples of halogens include fluorine, chlorine, bromine, and iodine.

The terms “heteroaromatic” and “heteroaryl” refer to a planar, mono- or polycyclic moiety with a continuous system of pi-conjugated atoms, composed of carbon and one or more elements independently selected from N, S, or O. Heteroaryl includes both monocyclic and fused polycyclic moieties ranging in size from 5 to 14 atoms. Heteroaromatic includes substituted and unsubstituted heteroaromatic groups. Preferably, a heteroaryl is a monocyclic moiety composed of 5 to 6 atoms and containing 1 to 4 heteroatoms selected from N, S, or O. More preferably, a heteroaromatic is a monocyclic moiety composed of six atoms and containing 1-2 nitrogen atoms. Examples of heteroaryl groups include thiophenyl, pyrrolo, furanyl imidazolyl, pyrazolyl, oxazolyl, isoxazolyl, thiazolyl, tetrazolo, 2-pyridyl, 3-pyridyl, 4-pyridyl, pyridono, pyridazinyl, pyrimidyl, pyrazinyl, triazinyl, dioxinyl, and thiazinyl.

The term “heteroatom” means an atom that is other than carbon or hydrogen. Examples of heteroatoms include N, O, S, Si, F, Cl, and P.

The terms “heterocyclic” and “heterocyclyl” refer to a cyclic moiety composed of carbon, hydrogen and one or more elements independently selected from N, S, or O. The term includes both monocyclic and fused polycyclic moieties ranging in size from 3 to 14 atoms. Preferably, a heterocyclic is a monocyclic moiety composed of 5 to 7 atoms and containing 1 to 4 heteroatoms selected from N, S, or O. Heterocyclic includes substituted and unsubstituted heterocyclic groups. Examples of heterocyclic groups include pyrrolidinyl, pyrrolinyl, pyrolidinyl, tetrahydrofuranyl, tetrahydrothiophenyl, oxazolidinyl, piperidinyl, piperazinyl, tetrahydropyranyl, dioxanyl, and morpholinyl.

The term “hydroxyl” refers to the group —OH.

The term “imino” refers to a trivalent carbon atom possessing a double bond to nitrogen and two single bonds to other elements in which one element is a component of the parent molecular group that the imino group is a substituent of and the other is selected from carbon, oxygen, nitrogen, sulfur, or hydrogen Imino includes substituted and unsubstituted imino groups. Examples of imino groups include ketimines, aldimines, imidates, thioimidate, amidines, oximes and hydrazones.

The term “nitro” refers to the group —NO₂.

The term “phosphate” refers to the group —OP(O)(OR)₂ or its anions, where each R is independently selected from hydrogen, alkyl, alkenyl, alkynyl, cycloalkyl, heterocyclyl, aryl, or heteroaryl. Phosphate includes substituted and unsubstituted phosphate groups.

The term “sulfinyl” refers to a group with the formula —S(O)— where the sulfinyl is bonded to the parent molecular group and a group selected from alkyl, alkenyl, alkynyl, cycloalkyl, heterocyclyl, aryl, or heteroaryl. Sulfinyl includes substituted and unsubstituted sulfonyl groups.

The term “substituted” is used herein to describe a compound or chemical moiety wherein at least one hydrogen atom of that compound or chemical moiety is replaced with a second chemical moiety. Non-limiting examples of substituents include alkenoxy, alkenyl, alkoxy, alkyl, alkylamino, alkynal, alkynoxy, amide, amidino, amino, aminoalkyl, aminoaryl, aryl, arylalkyl, aryloxy, azido, azo, carbamate, carbamide, carbonyl, carboxamide, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, thiol, and combinations thereof. These substituents can optionally be further substituted with a substituent selected from such groups. Substituents include, e.g., moieties in which a carbon atom is substituted with a heteroatom such as nitrogen, oxygen, silicon, phosphorous, boron, sulfur, or a halogen atom.

The term “sulfonamidyl” refers to a group with the formula —S(O₂)N— where the sulfonamidyl group is bonded to the parent molecular group through either the sulfur or the nitrogen. When the parent molecular group is bonded to the sulfur atom, the nitrogen of the group is optionally substituted with 0-2 groups independently selected from alkyl, alkenyl, alkynyl, cycloalkyl, heterocyclyl, aryl, or heteroaryl. When the parent molecular group is bonded to the nitrogen atom, the sulfur of the group is substituted with a group selected from alkyl, alkenyl, alkynyl, cycloalkyl, heterocyclyl, aryl, or heteroaryl and the nitrogen is optionally substituted with an additional group selected from alkyl, alkenyl, alkynyl, cycloalkyl, heterocyclyl, aryl, or heteroaryl.

The term “sulfonyl” refers to a group with the formula —S(O₂)— where the sulfonyl is bonded to the parent molecular group and a group selected from alkyl, alkenyl, alkynyl, cycloalkyl, heterocyclyl, aryl, or heteroaryl. Sulfonyl includes substituted and unsubstituted sulfonyl groups.

The term “thioalkyl” refers to an alkyl, cycloalkyl, heterocyclyl, alkenyl, or an alkynyl group bonded to the parent molecular group through a divalent sulfur atom. Thioalkyl includes substituted and unsubstituted thioalkyl groups.

The term “thioaryl” refers to an aromatic or heteroaromatic group bonded to the parent molecular group through a divalent sulfur atom. Thioaryl includes substituted and unsubstituted thioaryl groups.

The term “thiocarbonyl” refers to a trivalent carbon atom possessing a double bond to sulfur and two single bonds to other elements in which one element is a component of the parent molecular group that the thiocarbonyl group is a substituent of and the other is selected from carbon, oxygen, nitrogen, or sulfur. Thiocarbonyl includes substituted and unsubstituted thiocarbonyl groups. Examples of thiocarbonyl groups include thioketones, thioimidates, thioamides, and dithioesters.

The term “thioether” refers to an alkyl, cycloalkyl, heterocyclyl, alkenyl, or an alkynyl group bonded through a —C—S—C— linkage to another alkyl, cycloalkyl, heterocyclyl, alkenyl, or an alkynyl group. Ether includes substituted and unsubstituted ether groups.

The term “thiol” refers to the group —SH.

As used herein, the term “treat” and variations thereof, e.g., “treating”, “treatment”, refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

As used herein, the term “preventing” with respect to a condition or disorder refers to delaying or preventing the onset of such disorder or condition described herein, e.g., in a subject at risk of having the condition. In some embodiments, “preventing” a condition can also encompass inhibiting, decreasing, or slowing the progression or severity of the condition, e.g., in a subject being diagnosed with the condition. The onset, the progression, or severity of such disorder or condition can be determined by detecting an increase in at least one symptom associated with the condition, or a decrease in the function of the organ or organs affected by the condition.

The phrase “effective amount” or “therapeutically effective amount” as used herein refers to an amount of a compound described herein, or a composition comprising the compound, which is effective for producing some desired therapeutic effect in at least a sub-population of cells in a subject at a reasonable benefit/risk ratio applicable to any medical treatment. For example, a therapeutically effective amount of a compound or a composition comprising the compound can be an amount sufficient to produce a statistically significant, measurable change in at least one symptom of malaria as described herein.

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or which “consists of”) the named elements or steps to the exclusion of any other unnamed element or step. In any composition or method disclosed herein, known or disclosed equivalents of any named essential element or step may be substituted for that element or step. It is also understood that an element or step “selected from the group consisting of” refers to one or more of the elements or steps in the list that follows, including combinations of any two or more of the listed elements or steps.

The meaning of other terms will be understood by the context as understood by the skilled practitioner in the art, including the fields of organic chemistry, pharmacology, and microbiology.

### DETAILED DESCRIPTION OF THE INVENTION

Malaria is a parasitic infection of red blood cells caused by eukaryotic protists of the genus Plasmodium in the phylum Apicomplexa. Human malaria is known to be caused by five different Plasmodium species: Plasmodium falciparum, Plasmodium vivax, Plasmodium ovale, Plasmodium malariae, and Plasmodium knowlesi. Malaria parasites are transmitted by female Anopheles mosquitoes. After an initial cycle of replication in the liver, the parasites multiply within red blood cells, causing symptoms that include anemia (light headedness, shortness of breath, tachycardia), as well as other general symptoms such as enlarged spleen, fatigue, fever, chills, nausea, flu-like illness, and in severe cases, coma and death. According to the World Health Organization (WHO), in 2017, it was estimated that 435,000 deaths due to malaria had occurred globally, of which 403,000 deaths (approximately 93%) were in the WHO African Region. Almost 80% of all deaths in 2017 occurred in 17 countries in the WHO African Region and India. Therefore, currently there is an urgent need for a safe and effective method for the treatment or prevention of malaria.

Advantageously, the novel compounds as represented by Formula I, Formula I(a), and Formula I(b), and their methods of use as described herein are effective for treating human malaria caused by the above-referenced Plasmodium species. In particular, the present invention is directed to compositions and methods for treating malaria in a human subject caused by Plasmodium falciparum.

Without being limited to any particular theory or mode of action, it is believed that the novel compounds of the present invention inhibit or otherwise interfere with the ability of the malaria parasite to form the plasmodial surface anion channel (PSAC) with the host cell. PSAC plays a central role in the ability of the malaria parasite to acquire essential nutrients for survival and propagation in an infected RBC. Sugars, amino acids, purines, vitamins, and precursors for phospholipid biosynthesis have markedly increased uptake into infected RBC's via PSAC. Many of these solutes have negligible permeability in uninfected RBC's and must be provided exogenously to sustain in vivo parasite growth. PSAC is conserved on divergent plasmodial species. The channel's gating, voltage dependence, selectivity, and pharmacology are all conserved, suggesting that PSAC is a highly constrained integral membrane protein. Therefore, due to the conserved nature of PSAC across Plasmodium species, inhibition of PSAC formation is an attractive target for treating or preventing malaria and the compounds of the present invention should be effective for treating or preventing infection by most or all Plasmodium species.

Accordingly, the present invention is directed to a composition comprising novel pyridazinone compounds having the structure of Formula I:

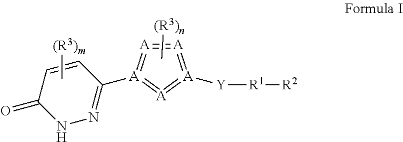

wherein:
A is independently selected from C, S, O or N combined through either single or double bonds to form a five-member heteroaromatic ring of 1-4 carbon atoms, 0-3 nitrogen atoms, 0-1 oxygen atom, and 0-1 sulfur atom;
R³ is a monovalent substituent group independently selected from alkenyl, alkoxy, alkyl, alkynal, having from 1 to 12 carbons, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halo, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent group is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, said substituent group may be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
n is an integer from 0-3;
m is an integer from 0-3;
Y is a divalent radical bridging A and R¹ selected from the group comprising, —COCH₂—, —SO₂—, —CO—, —CH₂—, —CH(CH₃)—, —NHCO—, —NCH₃CO—, —CONH—, —CONCH₃—, —O(CO)—, —(CO)O—, —NH—, or —O—;
R¹ is a divalent non-aromatic, heterocyclic ring of 5-7 members containing 0-2 nitrogen atoms, 0-1 oxygen atom, and 3-6 carbon atoms, with the proviso that Y and R² are separated by at least 3 atoms, which non-aromatic, heterocyclic ring may bear 0-3 substituent groups defined as for R³, with the proviso that two or more such substituent groups on R¹ may be fused with R¹ to form one or more cycloalkyl, heterocyclic, aromatic, or heteroaromatic rings, or alternatively R¹ may be fused, optionally incorporating 0-2 substituent groups, with R² to form a fused heterocyclyl ring of 3-7 members, optionally substituted with 0-2 substituent groups defined as for R³;
R² is a 5- or 6-membered heteroaryl ring bearing 0-4 substituent groups independently selected from substituent groups defined as for R³, or substituents on R² may be optionally fused to R² to form one or more cycloalkyl, heterocyclic, aryl or heteroaryl rings, or 0 -2 R² substituents may, together with R¹, form a fused substituted or unsubstituted heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cyclalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; or a pharmaceutically acceptable salt thereof.

In another embodiment, the present invention is directed to a composition comprising novel compounds having the structure of Formula I(a):

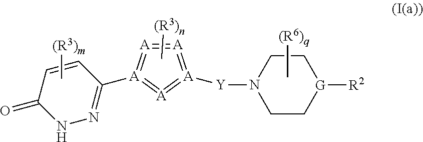

wherein:
G is selected from C or N and is part of a heterocyclic ring which is optionally substituted with (R⁶)_{q}, where q is an integer from 0-4; and
R⁶ is as defined for R³, with the additional proviso that R⁶ substituents on the heterocyclic ring containing G may be optionally fused to each other or a carbon atom of the ring containing G to form one or more cycloalkyl, heterocyclic, aromatic, or heteroaromatic rings; or 0-2 substituents on the heterocyclic ring containing G may, together with R², form a fused substituted or unsubstituted cycloalkyl or heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
R² is a 5- or 6-membered heteroaryl ring bearing 0-4 substituents independently selected from substituent groups defined as for R³, or substituents on R² may be optionally fused to R² to form one or more cycloalkyl, heterocyclic, aryl, or heteroaryl rings, of 3-8 members or a pharmaceutically acceptable salt thereof.

In another embodiment, the present invention is directed to a composition comprising novel compounds having the structure of Formula I(b):

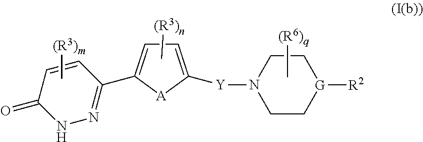

wherein:
A is independently selected from O, S or N, wherein,
n is an integer from 0-2 when A is O or S, and
n is an integer from 0-3 when A is N;
or a pharmaceutically acceptable salt thereof.

In another embodiment, compounds of the present invention are selected from:

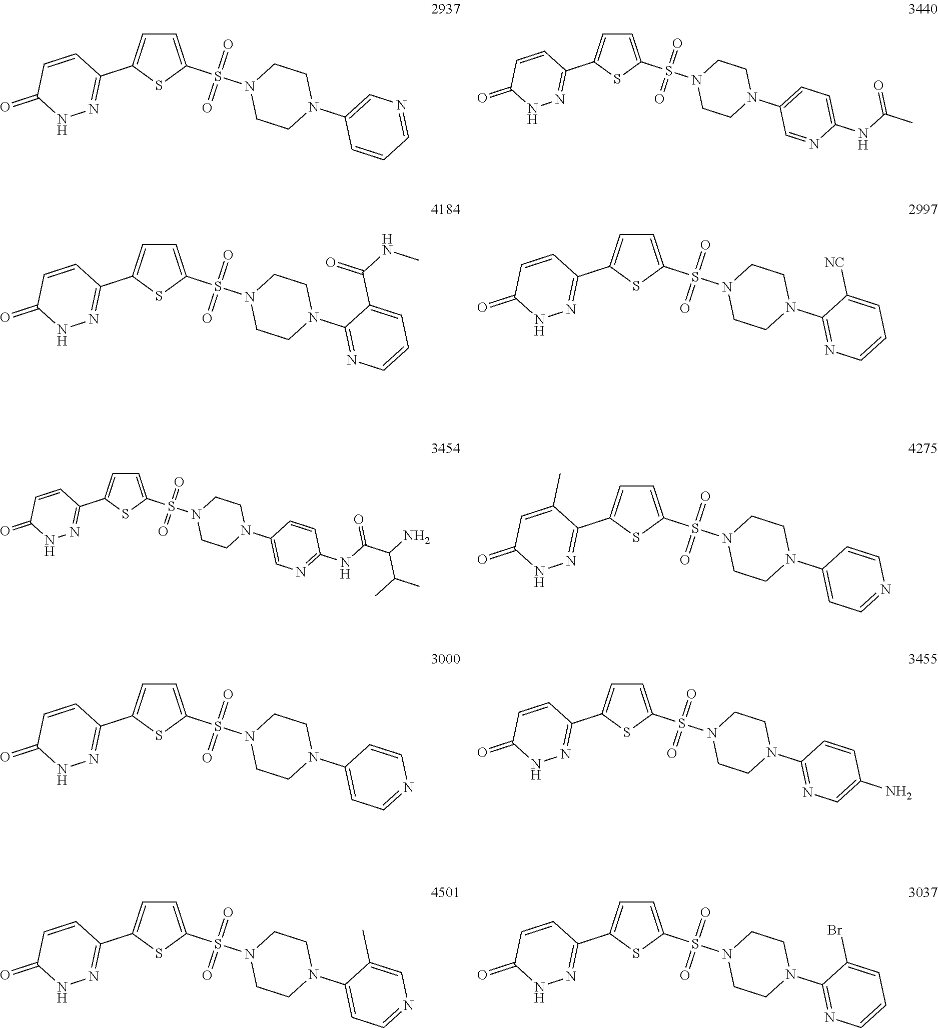

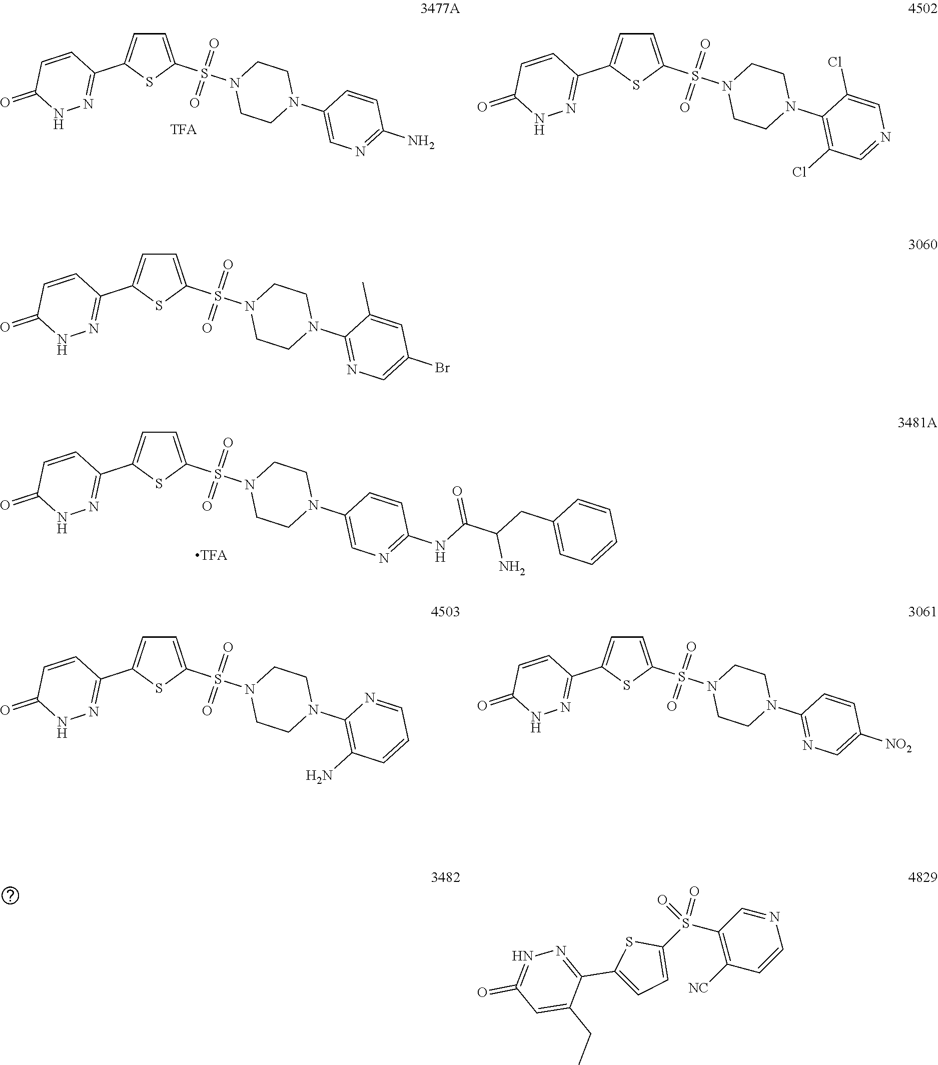

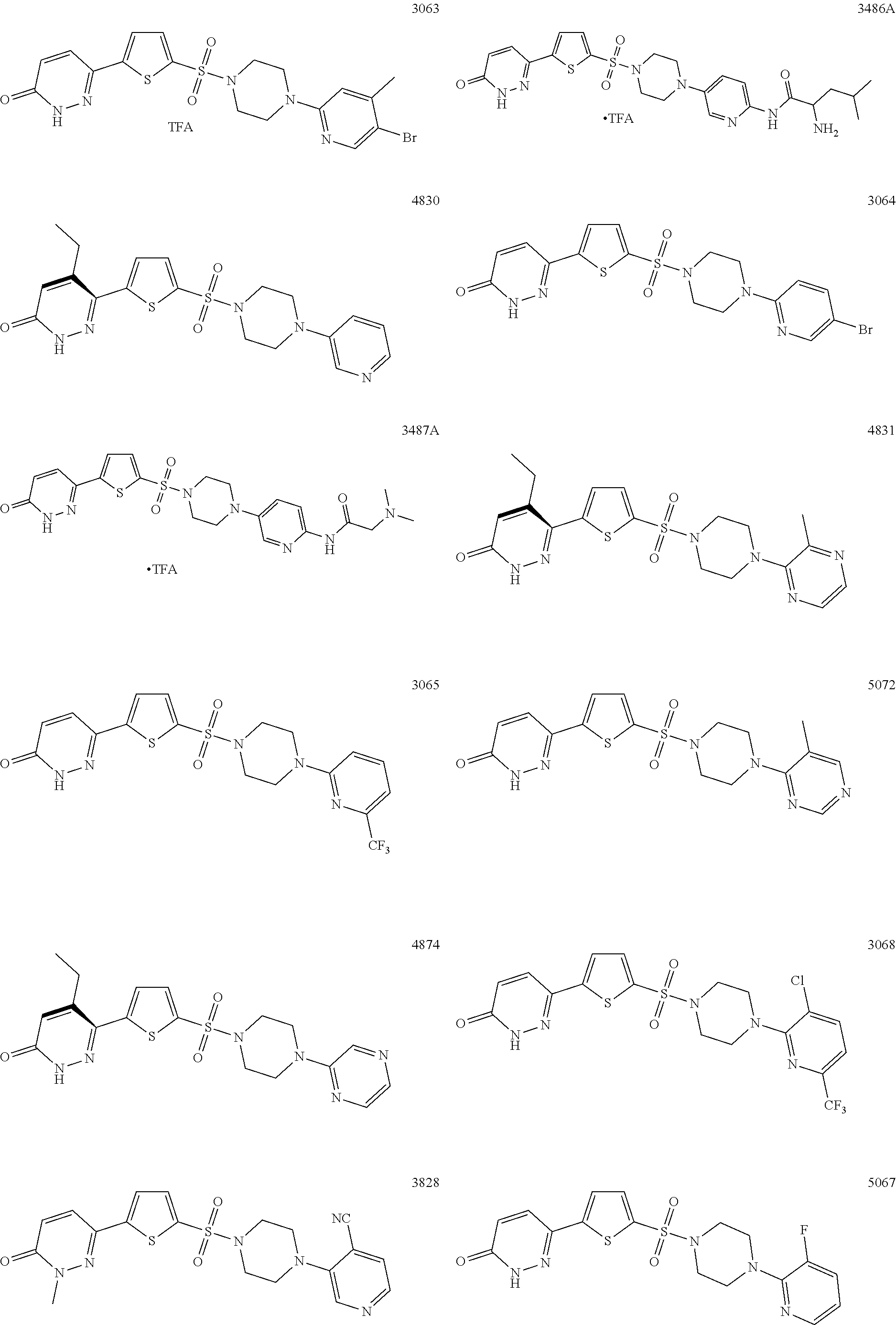

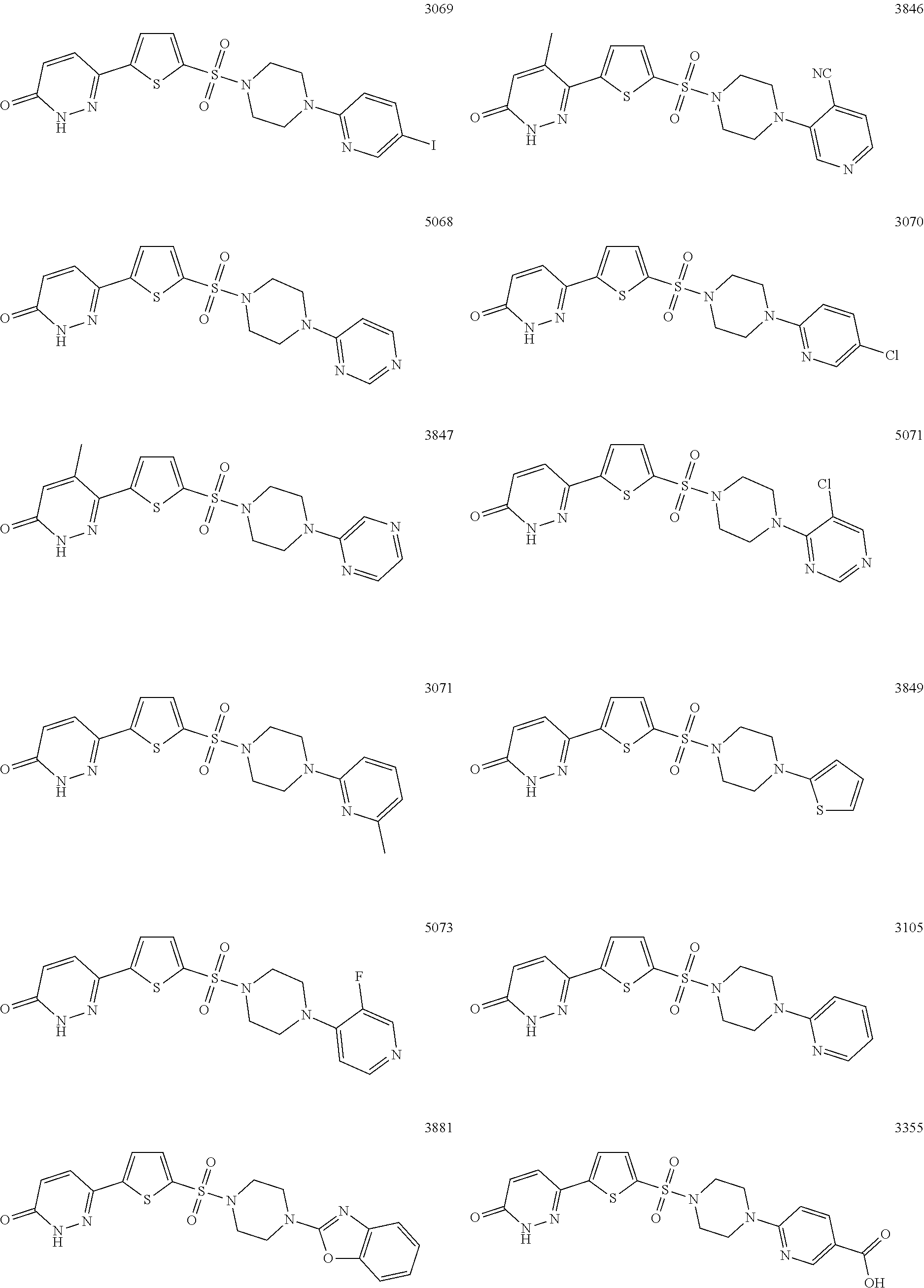

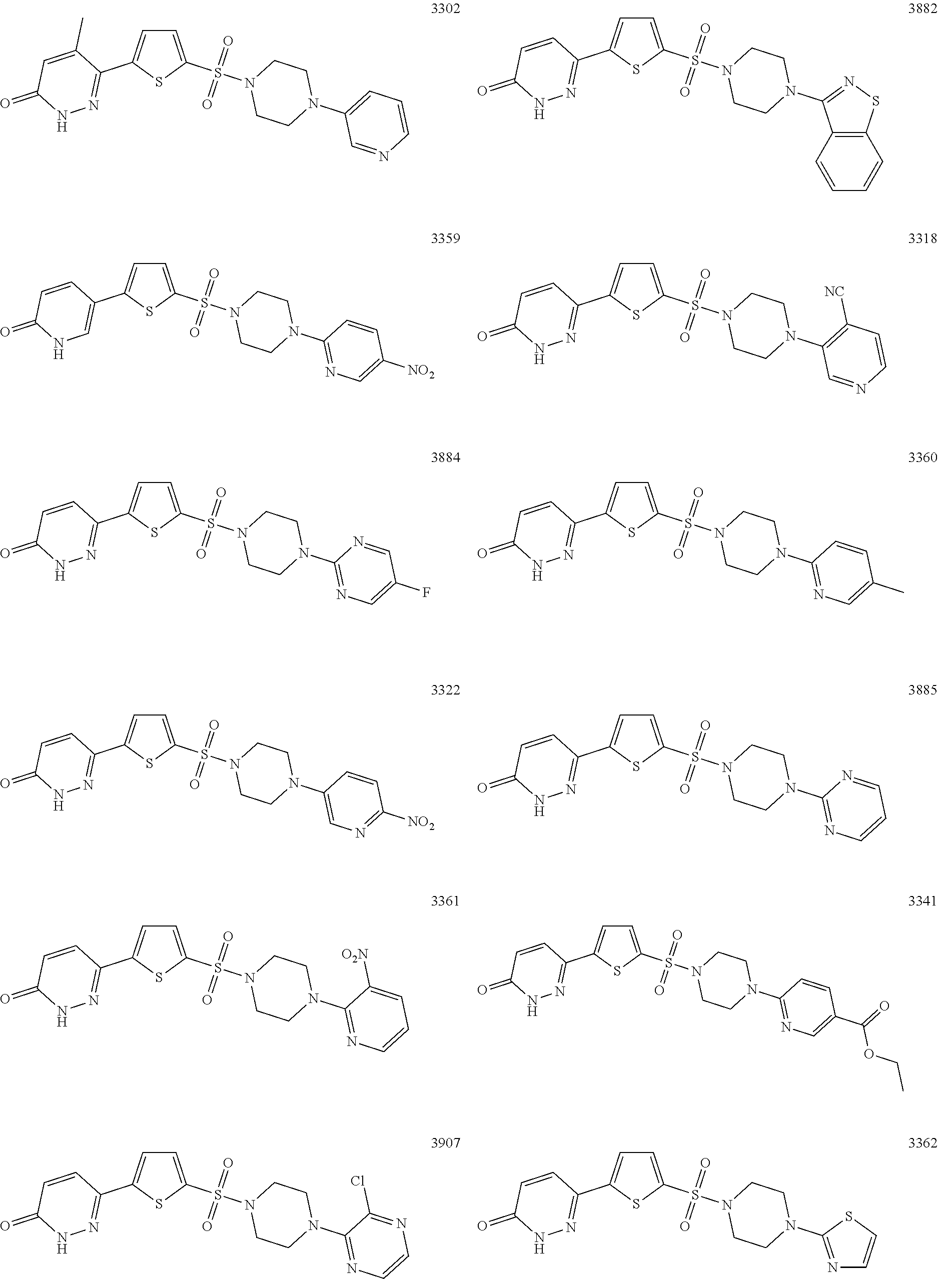

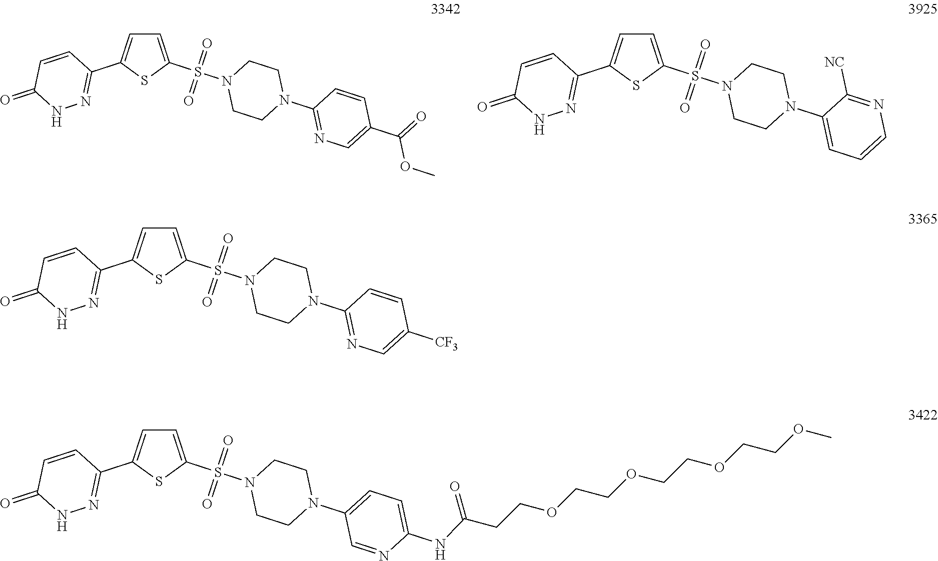 [SMILES: COC(=O)c1ccc(N2CCN(S(=O)(=O)c3ccc(-c4ccc(=O)[nH]n4)s3)CC2)nc1 ; N#Cc1ncccc1N1CCN(S(=O)(=O)c2ccc(-c3ccc(=O)[nH]n3)s2)CC1 ; O=c1ccc(-c2ccc(S(=O)(=O)N3CCN(c4ccc(C(F)(F)F)cn4)CC3)s2)n[nH]1 ; COCCOCCOCCOCCC(=O)Nc1ccc(N2CCN(S(=O)(=O)c3ccc(-c4ccc(=O)[nH]n4)s3)CC2)cn1]

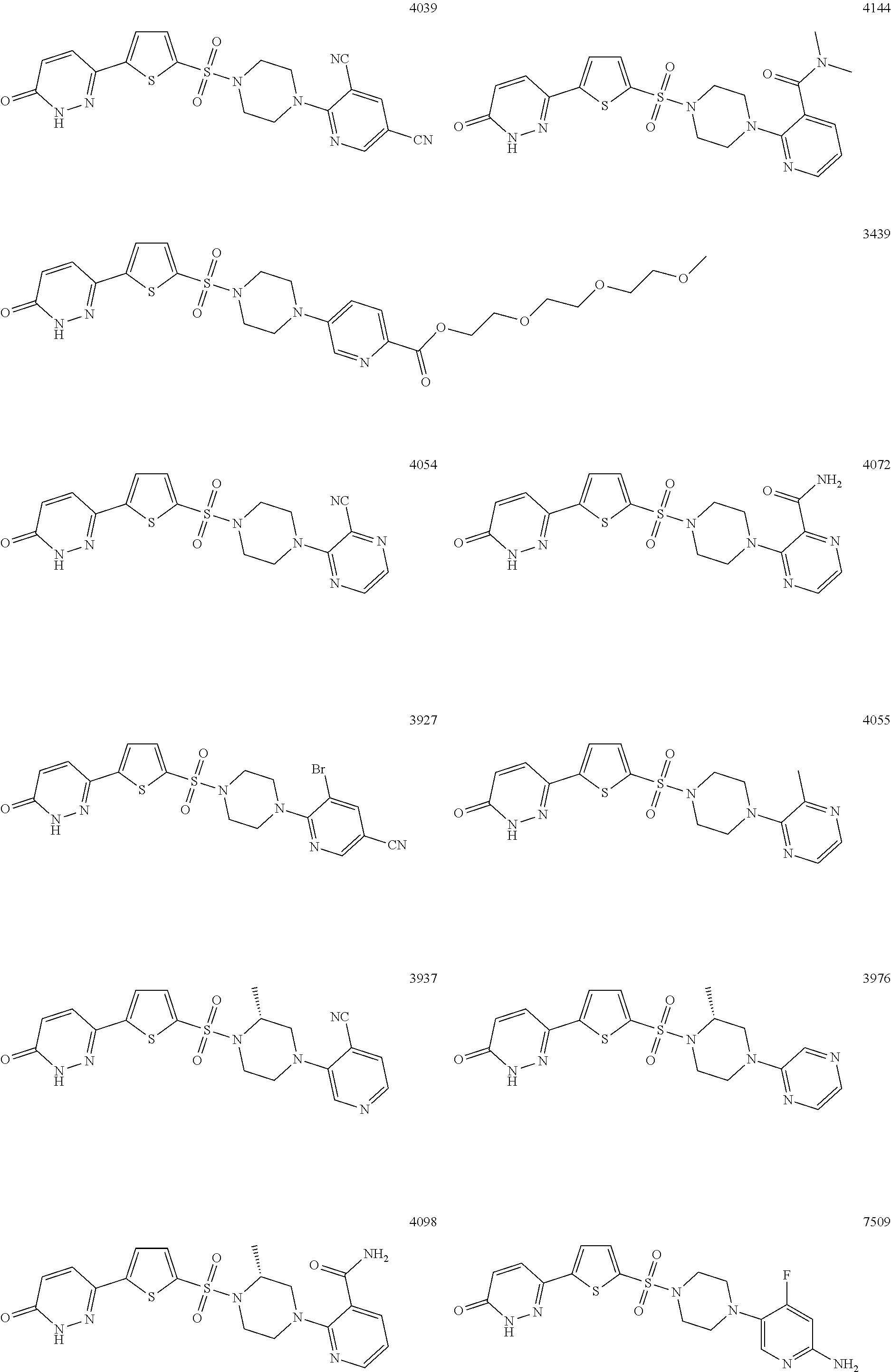

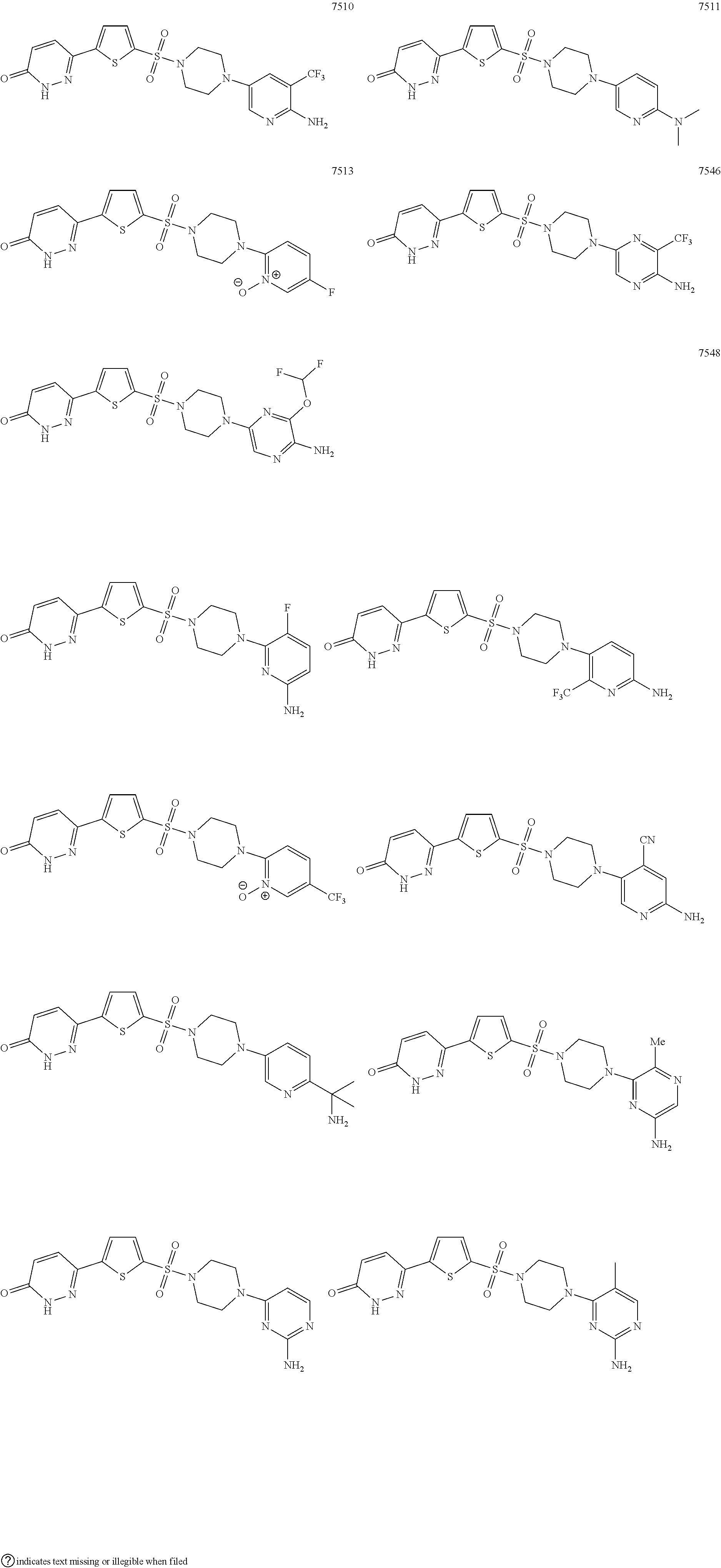

As described herein, it is demonstrated that the novel compounds of Formula I, Formula I(a), and Formula I(b) represent safe, potent, and effective anti-malaria compounds effective at treating or preventing this disease.

Therefore, in another embodiment, the present invention is directed to a method of treating or preventing malaria infection in a mammal comprising administering a composition comprising a therapeutically effective amount of at least one compound of Formula I, Formula I(a), and/or Formula I(b), in a pharmaceutically acceptable carrier, pharmaceutically acceptable salt, or excipient.

In another embodiment, the present invention is directed to a method of treating malaria infection in a mammal comprising administering a composition comprising any therapeutic combination of Formula I, Formula I(a), and/or Formula I(b) in a pharmaceutically acceptable carrier or excipient. For example, said composition may comprise a combination of compounds of Formula I and Formula I(a); Formula I and Formula I(b); Formula I, I(a), and I(b); Formula I(a) and I(b), etc. If administered in combination form, the compounds may be administered either simultaneously or serially in any order either immediately one after the other or at timed intervals.

In another embodiment, the present invention provides a pharmaceutical composition comprising the compound or compounds of Formula I, Formula I(a), and/or Formula I(b), and a pharmaceutically acceptable carrier. Pharmaceutically acceptable carriers such as vehicles, adjuvants, excipients, or diluents are well known to those skilled in the art. Preferably the pharmaceutically acceptable carrier is chemically inert to the active compound and has no detrimental side effects or toxicity under the conditions of use. The pharmaceutical formulations of the present invention are suitable for administration to an individual in need thereof via a variety of routes including oral, aerosol, parenteral, subcutaneous, intravenous, intraarterial, intramuscular, interperitoneal, intrathecal, rectal, and vaginal.

In another embodiment, the present invention provides a method of treating or preventing malaria in a mammal comprising administering an effective amount of a compound of Formula I, Formula I(a), and/or Formula I(b), and at least one other antimalaria compound or agent. Other antimalaria agents suitable for administration in conjunction with the novel compounds of the present invention include, but are not limited to, quinine, atovaquone, chloroquine, cycloguanil, hydroxychloroquine, amodiaquine, pyrimethamine, sulphadoxine, proquanil, mefloquine, halofantrine, pamaquine, primaquine, artemisinin, artemether, artesunate, artenimol, lumefantrine, dihydroartemisinin, piperaquine, artether, doxycycline, and clindamycin.

In another embodiment, the present invention provides a method of inhibiting formation of the malarial parasite plasmodial surface anion channel (PSAC) comprising administering a compound of Formula I, Formula I(a), and/or Formula I(b), optionally in combination with one or more additional antimalarial compounds or drugs.

According to the present invention, the pharmaceutically acceptable salt can include a pharmaceutically acceptable acid addition salt. The pharmaceutically acceptable acid addition salt can be obtained from inorganic acids such as hydrochloric acid, nitric acid, sulfuric acid, hydrobromic acid, hydroiodic acid, nitrous acid, or phosphorous acid, and nontoxic organic acids such as aliphatic mono- and di-carboxylates, phenyl-substituted alkanoate, hydroxyl alkanoate, and alkandioate, aromatic acids, and aliphatic and aromatic sulfuric acids.

Oral administration of the therapeutic compositions of the present invention in either solid or liquid form is advantageous since it represents a convenient and rapid method to administer a drug to a large, exposed population in case of pandemic. For oral administration, the pharmaceutical compositions may take the form of, for example, tablets or capsules prepared by conventional means with pharmaceutically acceptable carriers or excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art and may be formulated at sustained-release or controlled-released.

Liquid preparations for oral administration may take the form of, for example, solutions, syrups or suspensions or they may be presented as a dry product for constitution with water or other suitable vehicle before use. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol) and preservatives (e.g., methyl or propyl p-hydroxybenzoates or sorbic acid).

The inhibitors described herein will also be suitable for IV administration, because it is envisioned that in the case of a natural outbreak, the infected patients may require IV administration. Therefore, the inhibitors described herein will provide an effective, safe, and easy therapeutic option for any newly emerged pandemic strain(s).

The compounds of the present invention can be made into an aerosol formulation to be administered via inhalation. These aerosol formulations can be placed in pressurized acceptable propellants, such as dichlorodifluoromethane, propane, nitrogen and the like. They also may be formulated as pharmaceuticals for non-pressured preparation, such as in a nebulizer or atomizer.

Formulations suitable for parenteral administration may be formulated in unit dosage injectable forms and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizer, thickening agents, stabilizers, and preservatives. These particular formulations are especially suitable for intravenous, intramuscular, subcutaneous, and intraperitoneal administration.

The novel compounds of the present invention can be administered in a physiologically acceptable diluent in a pharmaceutical carrier, such as a sterile liquid or mixture of liquids, including water, saline, aqueous dextrose, and related sugar solutions, an alcohol, such as ethanol, isopropanol, or hexadecyl alcohol, lycols, such as propylene glycol or polyethylated glycol, glycerol ketals, such as 2,2-dimethyl-1,3-diosolane-4-methanol, ethers such as poly(ethyleneglycol) 400, an oil, a fatty acid, a fatty acid ester or glyceride, or an acylated fatty acid glyceride with or without the addition of a pharmaceutically acceptable surfactant, such as a soap or a detergent suspending agent, such as pectin, carbomers, methylcellulose, hydroxypropylmethylcellulose, or carboxymethylcellulose, or emulsifying agents and other pharmaceutical adjuvants.

The compounds of the present invention may be made into suppositories for rectal or vaginal administration by mixing with a variety of bases, such as emulsifying bases or water-soluble bases known in the art.

In one aspect, the invention relates to a kit comprising:
a) at least one compound according to Formulas I, I(a), and/or I(b), or a pharmaceutically acceptable salt, solvate, or polymorph thereof; and one or more of:
b) optionally at least one additional agent known to have antimalarial activity;
c) instructions for treating or preventing a malaria related disease;
d) instructions for administering the compound in connection with treating or preventing a malaria infection; or
e) instructions for administering the compound with at least one agent known to treat or prevent a malaria related disease.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound of the present invention and/or product and another component for delivery to a patient.

In a further aspect, the kit further comprises a plurality of dosage forms, the plurality comprising one or more doses; wherein each dose comprises an amount of the compound and the agent known to have antimalaria activity.

In a further aspect, an effective amount is a therapeutically effective amount. In a still further aspect, an effective amount is a prophylactically effective amount. The appropriate dose will depend upon several factors. For instance, the dose also will be determined by the existence, nature, and extent of any adverse side effects that might accompany the administration of a particular compound or salt. Ultimately, the attending physician will decide the dosage of the compound of the present invention with which to treat each individual patient, taking into consideration a variety of factors, such as age, body weight, general health, diet, sex, route of administration, and the severity of the disease condition.

In another embodiment, the present invention is directed to a pharmaceutical composition for treating or preventing malaria in a mammalian subject comprising a compound of Formula II:

Q-Y—R¹—R² (II)
wherein:
Q is a heteroaryl ring of 5 members having group Y bound to the ring at a non-adjacent site to a 6-pyridazin-3-(2H)-one, 5-pyridin-2(1H)-one, a substituted carboxamide, or a substituted carboxylate moiety, wherein Q is optionally substituted on either the heteroaryl ring, the 6-pyridazin-3-(2H)-one moiety, or both, with one or more substituent groups independently selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, and thiocarbonyl, each substituent group may be optionally substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, and thiol;
Y is a divalent radical bridging Q and R¹ selected from the group comprising: —COCH₂—, —CH₂CO—, —SO₂—, —CO—, —CH₂—, —CH(CH₃)—, —NHCO—, —NCH₃CO—, —CONH—, —CONCH₃—, —O(CO)—, —(CO)O—, —NH—, and —O—;
R¹ is a divalent non-aromatic heterocyclic ring of between 5-7 members containing 0-2 nitrogen atoms, 0-1 oxygen atoms, and 3-6 carbon atoms, with the proviso that Y and R² are separated by at least 3 atoms, which non-aromatic, heterocyclic ring may bear 0-3 substituent groups selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when the substituent group is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, each substituent can be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol, with the proviso that two or more such substituent groups on R¹ may be fused with R¹ to form one or more cycloalkyl or heterocyclic rings, or alternatively R¹ may be fused with R² to form a fused cycloalkyl or heterocyclyl ring of 3-7 members, optionally substituted with 0-2 substituent groups selected from alkenoxy, alkenyl, alkoxy, alkyl, alkylamino, alkynal, alkynoxy, amide, amidino, amino, aminoalkyl, aminoaryl, aryl, arylalkyl, aryloxy, azido, azo, carbamate, carbamide, carbonyl, carboxamide, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; and
R² is a 5- or 6-membered heteroaryl ring bearing 0-4 substituent groups independently selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, said substituent group may be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; or
substituents on R² may be optionally fused to R² to form one or more cycloalkyl, heterocyclic, aryl or heteroaryl rings; or 0-2 R² substituents may, together with R¹, form a fused substituted or unsubstituted cycloalkyl or heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
or a pharmaceutically acceptable salt thereof.

Compounds according to Formula II include the following:

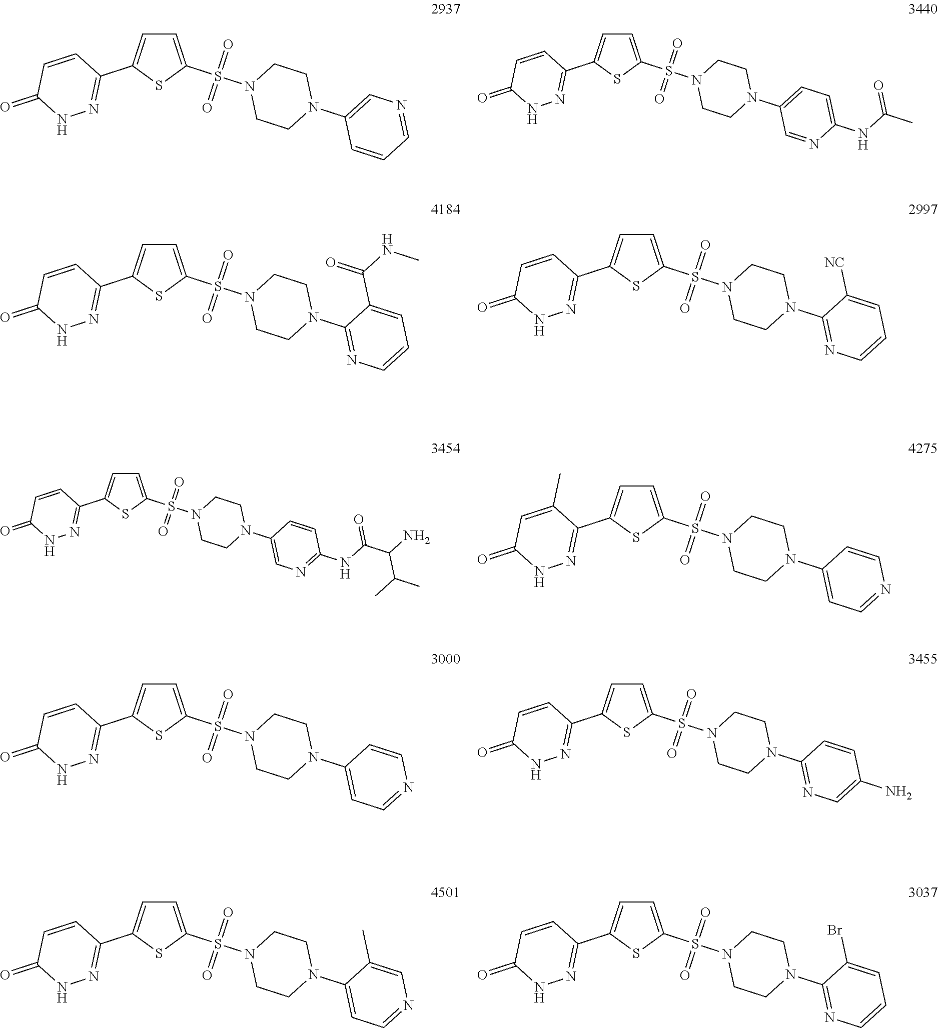

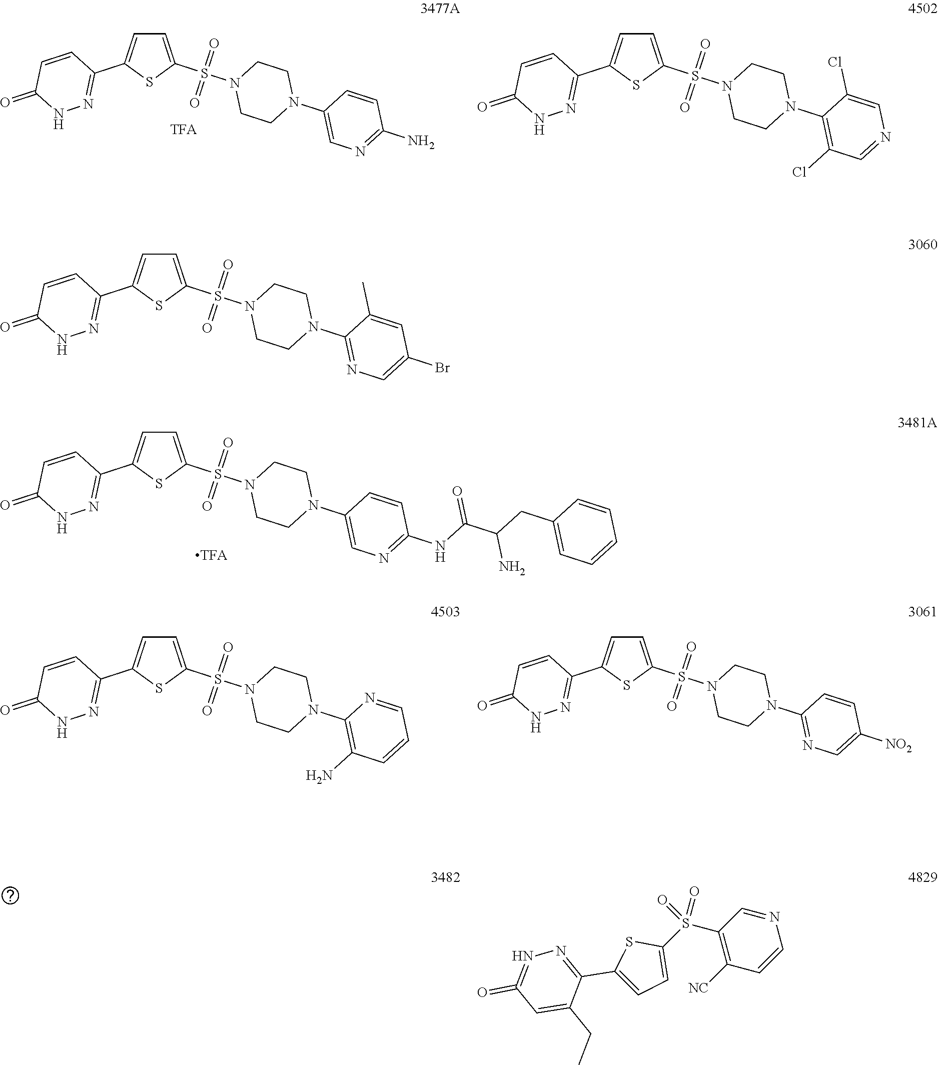

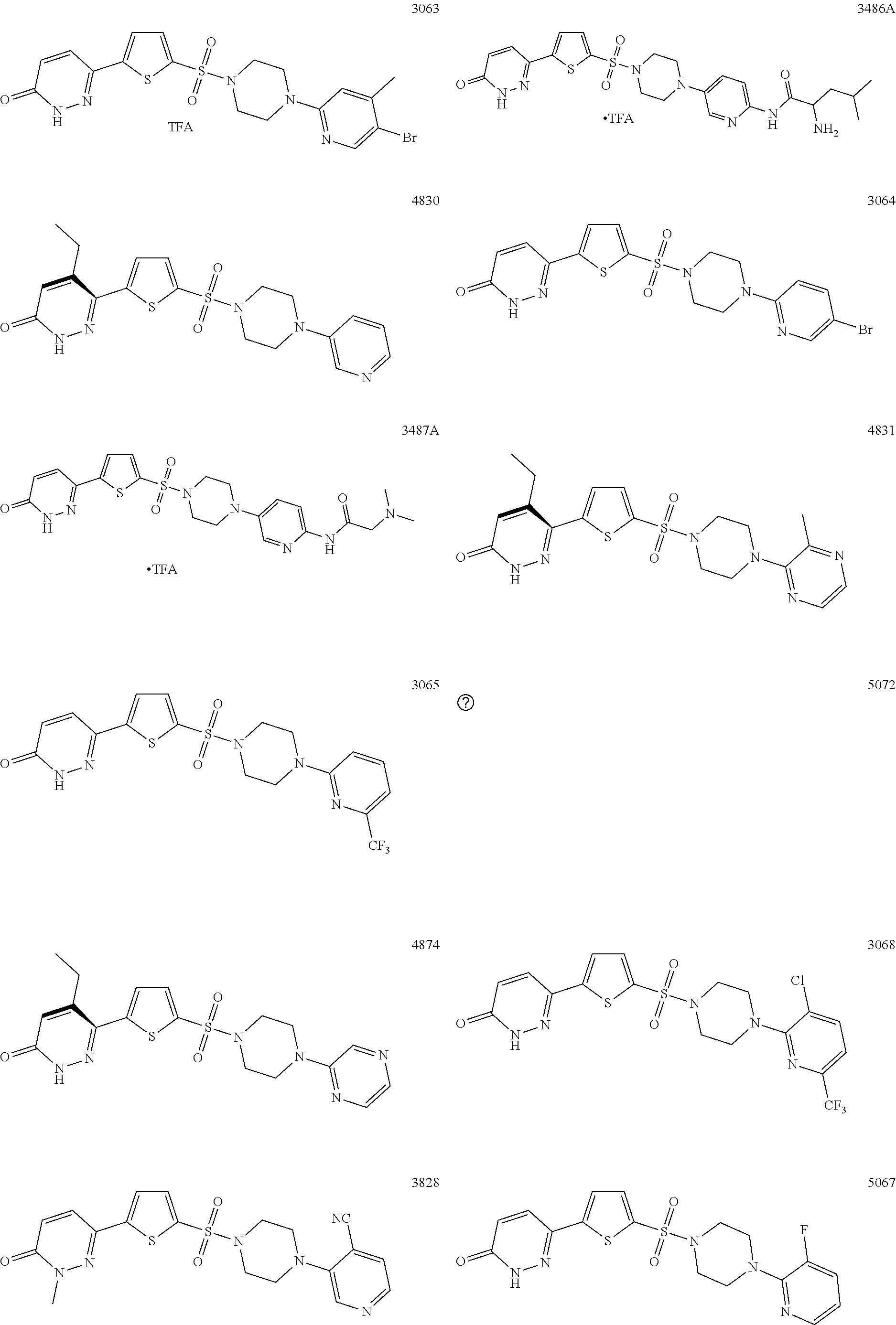

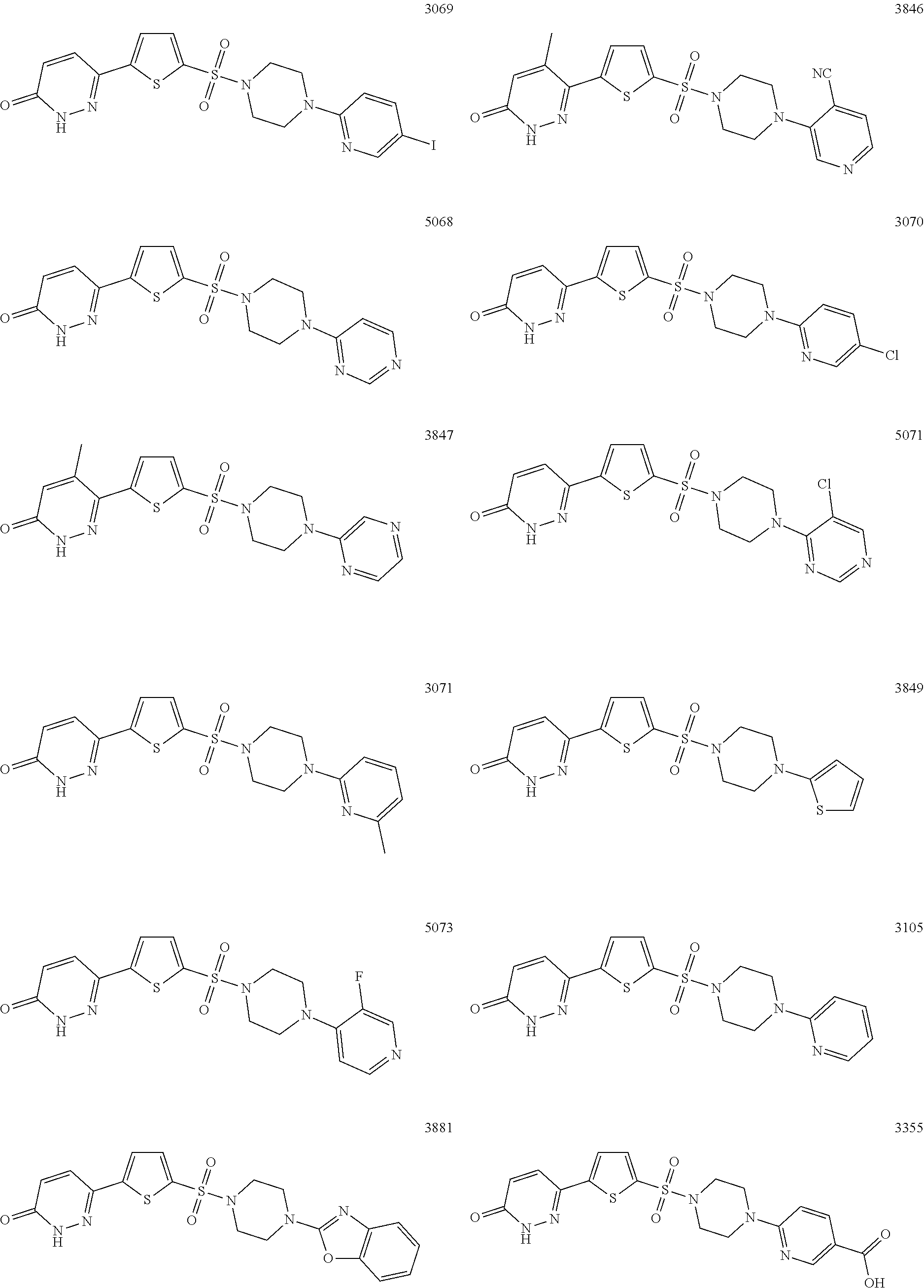

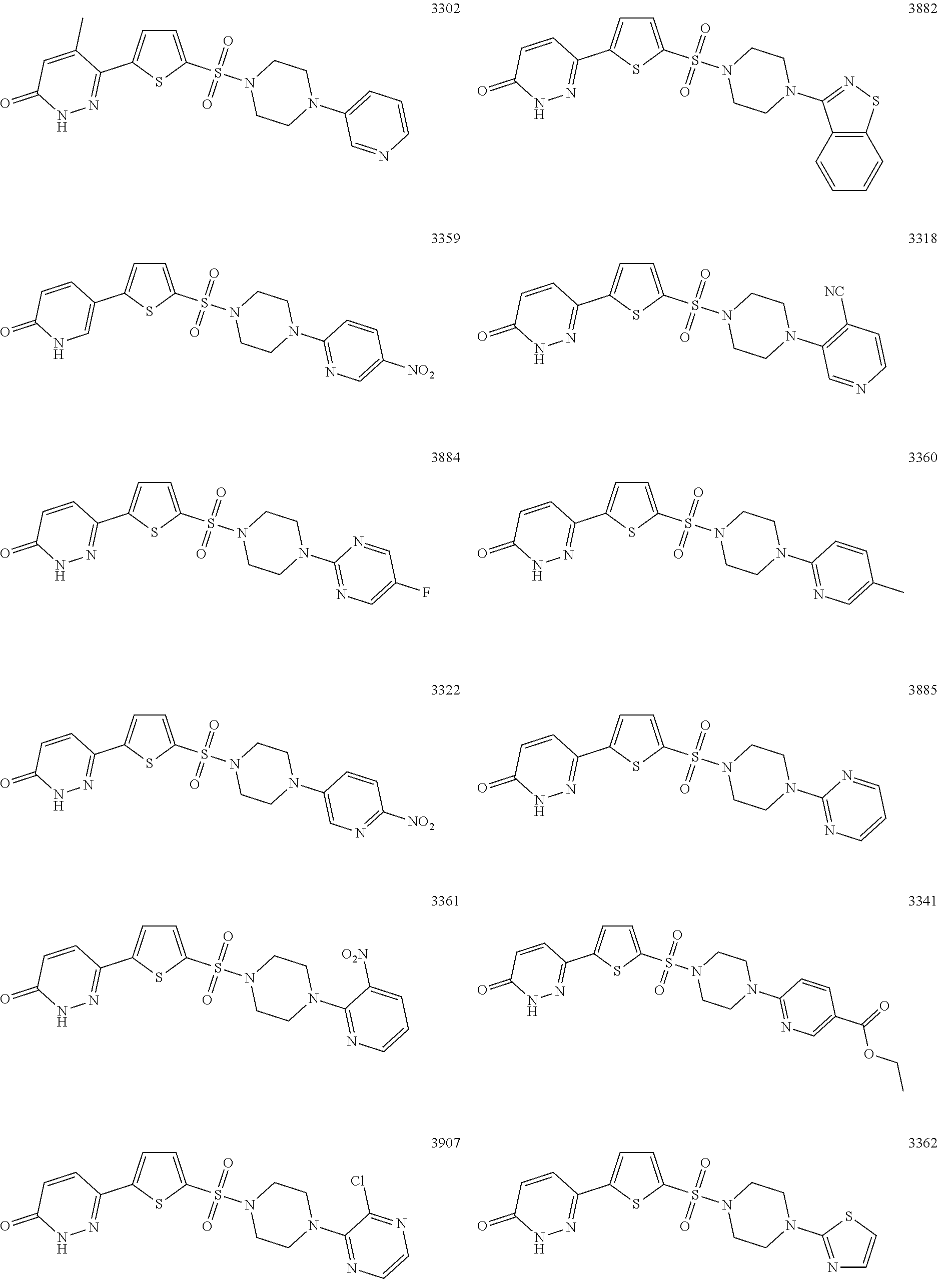

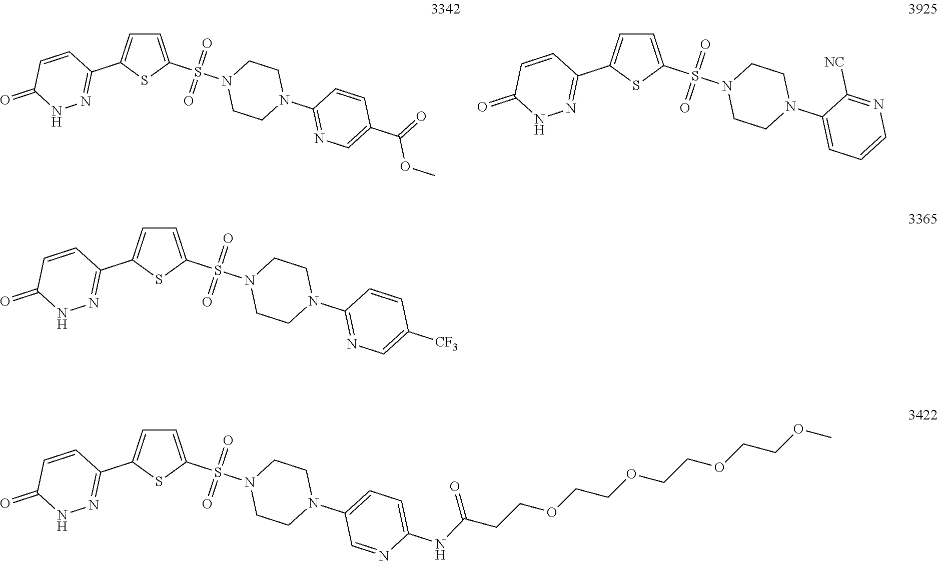 [SMILES: COC(=O)c1ccc(N2CCN(S(=O)(=O)c3ccc(-c4ccc(=O)[nH]n4)s3)CC2)nc1 ; N#Cc1ncccc1N1CCN(S(=O)(=O)c2ccc(-c3ccc(=O)[nH]n3)s2)CC1 ; O=c1ccc(-c2ccc(S(=O)(=O)N3CCN(c4ccc(C(F)(F)F)cn4)CC3)s2)n[nH]1 ; COCCOCCOCCOCCC(=O)Nc1ccc(N2CCN(S(=O)(=O)c3ccc(-c4ccc(=O)[nH]n4)s3)CC2)cn1]

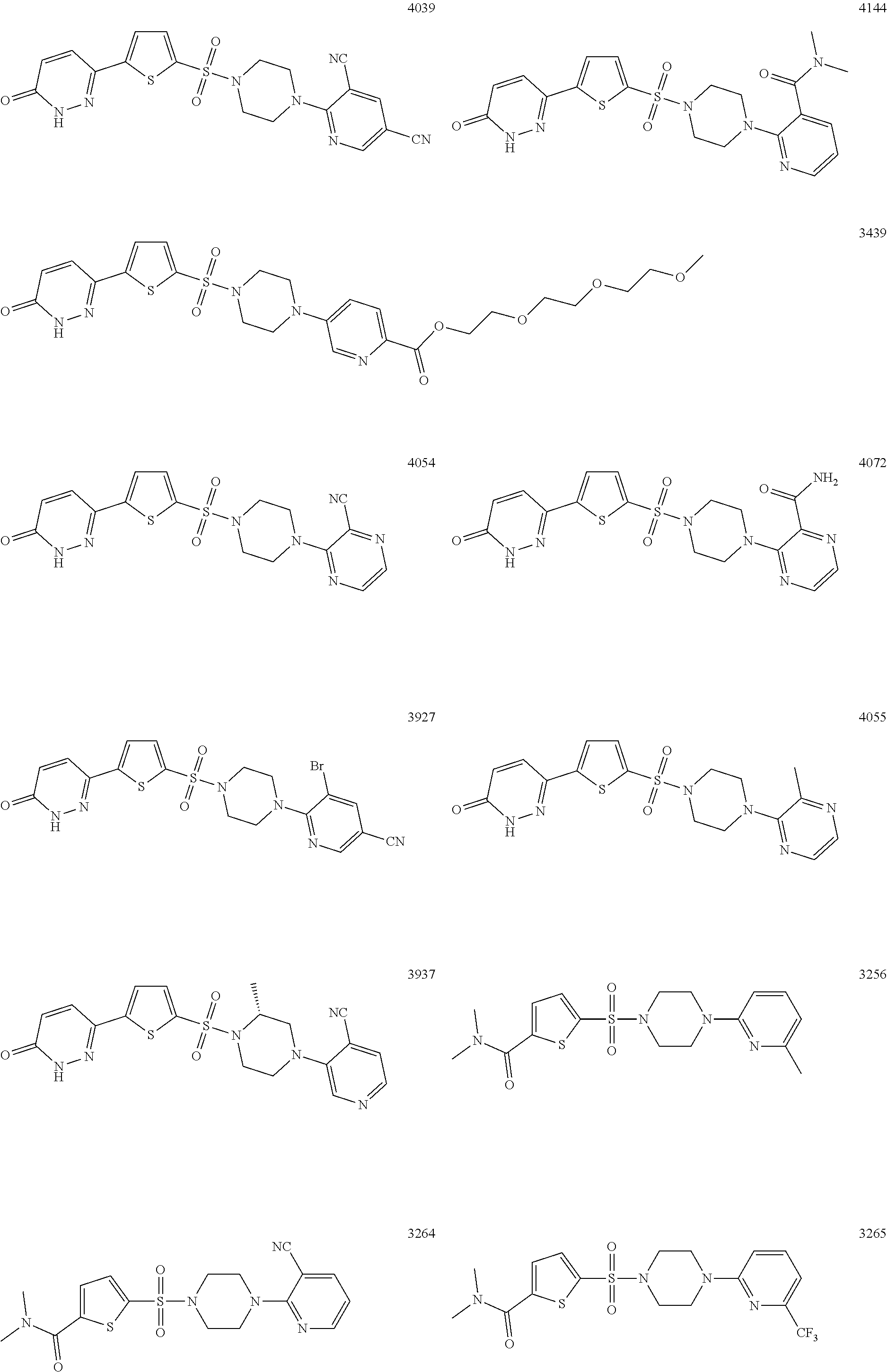

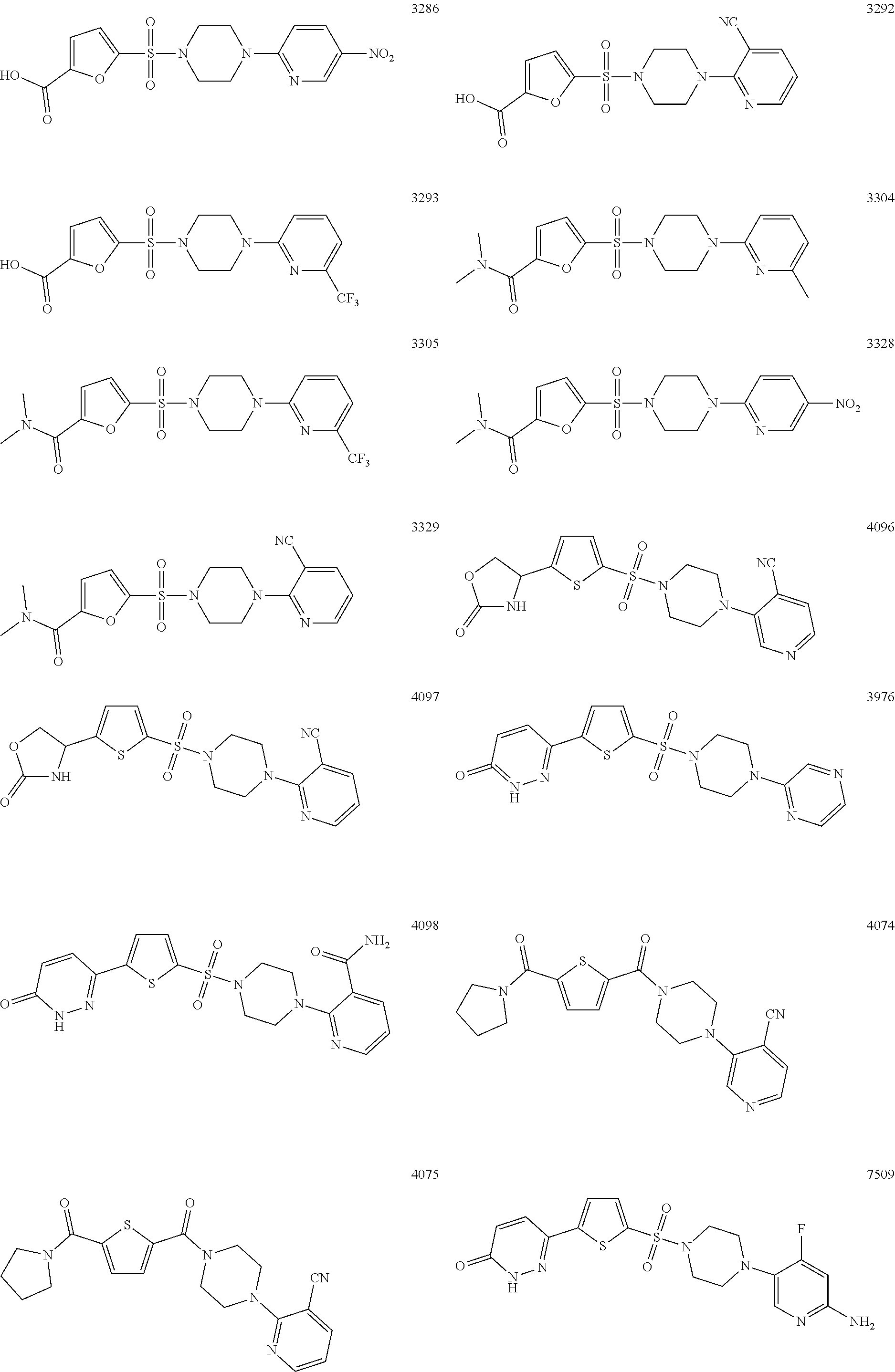

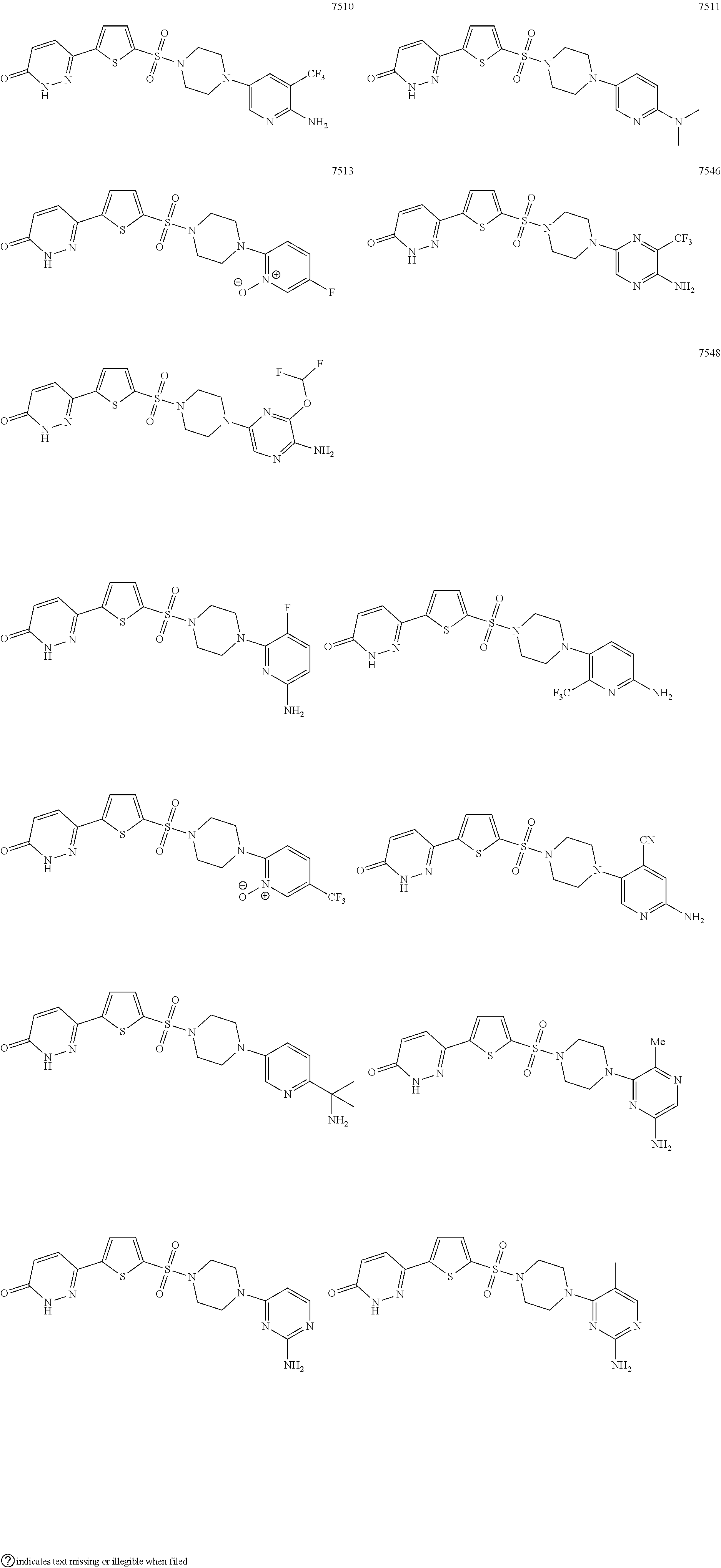

In another embodiment, the present invention is directed to a method for treating or preventing malaria in a mammalian subject comprising administering to said subject an effective amount of a pharmaceutical composition comprising compounds of Formula II. In a preferred embodiment, the mammal is a human.

In another embodiment, the pharmaceutical composition comprising compounds of Formula II may be administered in combination with a second antimalarial agent.

In another embodiment, the present invention is directed to the use of a pharmaceutical composition comprising Formula II for treating or preventing malaria infection in a mammalian subject. In a preferred embodiment, the mammal is a human.

In another embodiment, the present invention is directed to the use of a pharmaceutical composition comprising Formula II in the manufacture of a medicament for treating or preventing malaria infection in a mammalian subject. In a preferred embodiment, the mammal is a human.

### Examples

The following Examples have been included to illustrate modes of the presently disclosed subject matter. In light of the present disclosure and the general level of skill in the art, those of skill will appreciate that the following Examples are intended to be illustrative of the invention and are not intended to limit the scope of the invention as described in this application.

### Example 1. Synthesis of Malaria Inhibitor Compounds

Pyradazinyl compounds as described herein may be synthesized by the following general scheme:

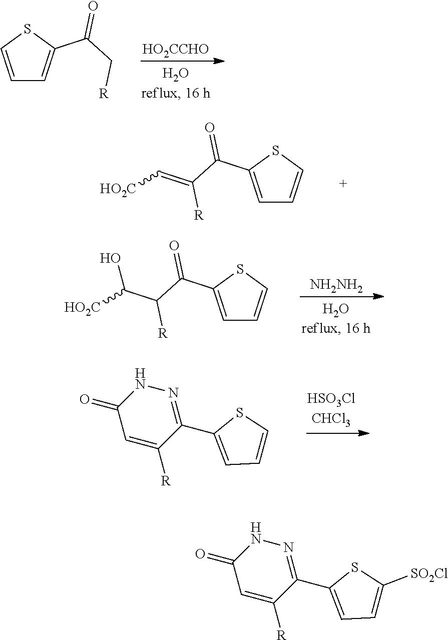

Compounds of Formula I may be synthesized as follows:

### General Procedure for Pyradazinyl thiosulfonyl chloride Formation: Synthesis of 5-(4-methyl-6-oxo-1,6-dihydropyridazin-3-yl)thiophene-2-sulfonyl chloride

Step 1: A flask containing glyoxylic acid monohydrate (4.17 g, 45.3 mmol) and 1-(thiophen-2- yl)propan-1-one (19.0 g, 135 mmol) in water (20 mL) was heated at 130° C. for 16 h. The mixture was cooled to room temperature and neutralized by adding a mixture of water (20 mL) and NH₄OH (3 mL, 28-30% aqueous solution). The excess remaining 1-(thiophen-2-yl)ethenone was then removed by extraction with CH₂Cl₂ (10 mL×2). Hydrazine hydrate (2.2 mL) was added to the resulted aqueous layer and the mixture was heated at reflux for 16 hrs. After cooling to room temperature, the liquid was decanted off and methanol (3 mL) was added to the remaining waxy solid. After filtering the resulting suspension, the solid was rinsed with methanol and dried in vacuo to yield 5-methyl-6-(thiophen-2-yl)pyridazin-3(2H)-one as a yellow solid (2.55 g, 29%); ¹H NMR (DMSO-d6): δ13.14; (br s, 1H), 7.64; (d, 1H), 7.48; (d, 1H), 7.50; (dd, 1H), 6.83; (s, 1H), 2.37; (s, 3H); MS: 193.0 (M+1).

Step 2: To a flask containing CHCl₃ (10 mL), cooled in an ice bath, was slowly added ClSO₃H (1 mL). After 10 min, 5-methyl-6-(thiophen-2-yl)pyridazin-3(2H)-one (0.69 g, 3.6 mmol) was added in portions over 30 min The reaction mixture was allowed to warm to room temperature. After 8 h, the reaction mixture was poured onto ice. The resultant yellow solid was collected by filtration, rinsed with water and dried in vacuo to yield 5-(4-methyl-6-oxo-1, 6-dihydropyridazin-3-yl)thiophene-2-sulfonyl chloride as a yellow solid (0.7 g, 67%, >98% purity); ¹H NMR (DMSO-d6): δ13.09; (br s, NH), 7.26; (d, 1H), 7.09; (d, 1H), 6.83; (s, 1H), 2.36; (s, 3H); MS: 291.1 (M+1). Other sulfonyl chlorides were either prepared in this manner or obtained from commercial sources.

### General Procedure for Sulfonamide Formation: 2-(4-((5-(6-oxo-1,6-dihydropyridazin-3-yl)thiophen-2-yl)sulfonyl)piperazin-1-yl)nicotinonitrile MBX 2997

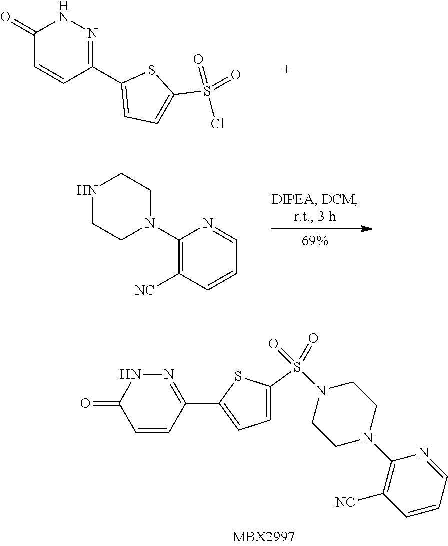 [SMILES: O=c1ccc(-c2ccc(S(=O)(=O)Cl)s2)n[nH]1 ; N#Cc1cccnc1N1CCNCC1 ; N#Cc1cccnc1N1CCN(S(=O)(=O)c2ccc(-c3ccc(=O)[nH]n3)s2)CC1]

To a flask containing 5-(6-oxo-1,6-dihydropyridazin-3-yl)thiophene-2-sulfonyl chloride (150 mg, 0.54 mmol) and 2-(piperazin-1-yl)nicotinonitrile (131 mg, 0.54 mmol) in dichloromethane (8 mL) was added diisopropylethylamine (0.14 mL, 0.81 mmol), and stirred at room temperature under argon atmosphere for 2 hrs. The resultant heterogenous mixture was filtered and washed with dichloromethane several times and dried in in vacuo (12 hrs) to provide a white solid (170 mg, 65%). ¹H NMR (300 MHz, DMSO-d6): δ13.30; (s, 1H), 8.42; (dd, 1H), 8.13-8.08; (m, 2H), 7.85; (d, 1H), 7.72; (d, 1H), 7.08-6.96; (m, 2H), 3.70-3.60; (m, 4H), 3.20-3.05; (m, 4H); LCMS (429.3 (M+1).

Other sulfonamides were prepared in a similar manner from appropriate starting materials. Piperazines used in this step were either obtained from commercial sources or generated using standard chemical procedures from the literature.

Compounds of Formula II may be synthesized as follows:

Carboxamide compounds as described herein may be synthesized by the following general scheme:

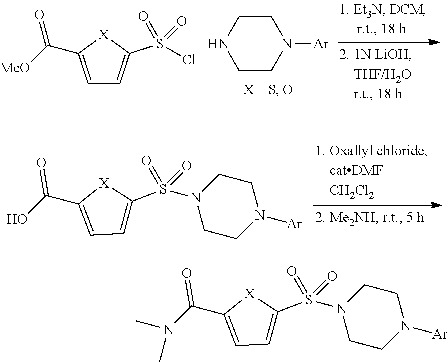

### General Procedure for Sulfonyl thiophene carboxamide Formation: 5-((4-(2,5-dimethylphenyl)piperazin-1-yl)sulfonyl)-N,N-dimethylthiophene-2-carboxamide (MBX-3248)

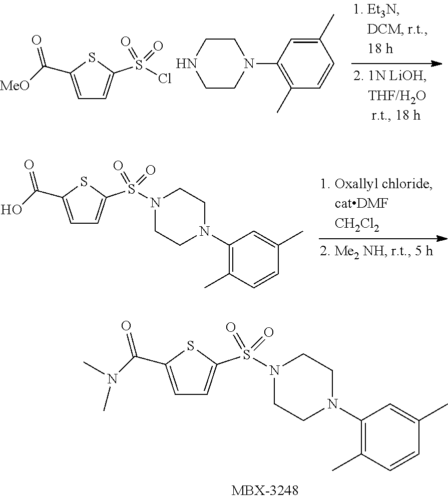

Step 1: Triethylamine (0.120 mL, 1.3 eq) was added to a flask containing methyl 5-(chlorosulfonyl)thiophene-2-carboxylate (150 mg, 0.67 mmol, 1.0 eq) and 1-(2,5-dimethylphenyl) piperazine (153 mg, 0.80 mmol, 1.2 eq) in dichloromethane (4.0 mL); this reaction was stirred at room temperature for 18 h. The mixture was then diluted with water (10 mL) before extracting the aqueous layer with CH₂Cl₂ (2×10 mL). The combined organics were dried over anhydrous Na₂SO₄, filtered and concentrated under reduced pressure and dried under vacuum to give desired product quantitatively as a yellow solid. The resultant compound (295 mg, 0.67 mmol, 1.0 equiv.) was dissolved in THF/H₂O (1:1, 0.04 M) and to it was added 21.0 mL of 1 N LiOH(aq) and stirred at room temperature for 18 h. The volatiles were removed and the resultant aqueous layer was acidified to pH˜2 using 2 N HCl. The formed precipitate was filtered and washed with hexanes (3×5 mL) and dried in vacuo to yield 0.306 g of product, which was used for the next reaction without further purification, MS: 381 (M+1). ¹H NMR (DMSO-d6): δ7.84; (d, 1H), 7.70; (d, 1H), 7.00; (d, 1H), 6.89; (s, 1H), 6.78; (d, 1H), 3.34; (s, 4H), 2.93; (s, 4H), 2.23; (s, 3H), 2.10; (s, 3H).
Step 2: To a solution of 5-((4-(2,5-dimethylphenyl)piperazin-1-yl)sulfonyl)thiophene-2-carboxylic acid (306 mg, 0.80 mmol, 1.0 eq) in CH₂Cl₂ (0.15 M) was added DMF (0.50 mL, 3.0 eq) followed by cooling in an ice-water bath. Oxalyl chloride (0.3 mL, 2.0 eq) was then added. After warming to room temperature and stirring for 1 h, the reaction mixture was bubbled with Me₂NH gas for a few minutes and stirred at room temperature from 1-5 h. The reaction was monitored by TLC. After the reaction was complete, water (10 mL) was added and the organics were separated. The aqueous layer was extracted with CH₂Cl₂ (2×10 mL). All the organics were combined, dried over anhydrous Na₂SO₄, filtered and concentrated to give crude product, which was purified using a silica gel column with EtOAc/DCM 0-40% gradient). Desired fractions were combined and concentrated under reduced pressure and dried in vacuo to yield pure desired product as a white solid (225 mg, 69%). ¹H NMR (300 MHz, CDCl₃) δ7.49; (d, 1H), 7.36; (d, 1H), 7.06; (d, 1H), 6.84- 6.81; (m, 2H), 3.30-3.20; (m, 4H), 3.20-3.15; (m, 6H), 3.02-2.99; (m, 4H), 2.30; (s, 3H), 2.18; (s, 3H); MS 408.0 (M+1); Rf: 0.50; (3% MeOH/CH₂Cl₂).

Other carboxamides were prepared in a similar manner from appropriate starting materials. Piperazines used in this step were either obtained from commercial sources or generated using standard chemical procedures from the literature.

### Example 2. Characterization Data of Select Malaria Inhibitors

Novel malaria inhibitor compounds were characterized by ¹H NMR spectra at 300 MHz and LCMS with the m/z (typically M+1) using an electrospray ionization strategy. Tables 5 and 6 (below) present representative data for select examples.

TABLE 5
NMR Data of Select Malaria Inhibitors of Formula 1

| | | m/z found |
|---|---|---|
| Cmpd No. | | by LCMS |
| MBX- | ¹H NMR Spectrum (solvent) | (M + x) |
| 2937 | (CDCl3:DMSO, 2:1): 10.32 (bs, 1H), 8.16 (m, 1H), 7.78 (m, 1H), | 417.2 |
| | 7.59 (d, 1H), 7.55-7.48 (m, 2H), 7.43-7.37 (m, 2H), 7.31 (d, 1H), 7.15 | (M + 1) |
| | (dd, 1H), 6.70-6.66 (m, 2H), 3.81 (bs, 2H), 3.56 (bs, 6H) | |
| 2997 | (DMSO) 13.30 (s, 1H), 8.42 (dd, 1H), 8.13-8.08 (m, 2H), 7.85 (d, | 429.3 |
| | 1H), 7.72 (d, 1H), 7.08-6.96 (m, 2H), 3.70-3.60 (m, 4H), 3.20-3.05 | (M + 1) |
| | (m, 4H) | |
| 3000 | (DMSO): 13.30 (bs, 1H), 8.15 (d, 2H), 8.09 (d, 1H), 7.83 (d, 1H), | 404.4 |
| | 7.69 (d, 1H), 7.03 (d, 1H), 6.81 (d, 2H), 3.49 (m, 4H), 3.08 (m, 4H) | (M + 1) |
| 3037 | (DMSO) 13.31 (s, 1H), 8.27 (dd, 1H), 8.14 (d, 1H), 7.99 (dd, 1H), | 482.5 |
| | 7.87 (d, 1H), 7.72 (d, 1H), 7.06 (d, 1H), 6.99 (dd, 1H), 3.36-3.20 (m, | (M + 1) |
| | 4H), 3.20-3.10 (m, 4H) | |
| 3060 | (DMSO) 13.30 (s, 1H), 8.18 (d, 1H), 8.14 (d, 1H), 7.86 (d, 1H), 7.75 | 496.5 |
| | (d, 1H), 7.70 (d, 1H), 7.06 (d, 1H), 3.22-3.10 (m, 8H), 2.16 (s, 3H) | (M + 1) |
| 3061 | (DMSO) 13.30 (s, 1H), 8.94 (d, 1H), 8.25 (dd, 1H), 8.11 (d, 1H), 7.82 | 449.2 |
| | (d, 1H), 7.69 (d, 1H), 7.05 (dd, 1H), 6.95 (d, 1H), 4.00-3.80 (m, 4H), | (M + 1) |
| | 3.20-3.00 (m, 4H) | |
| 3063 | (DMSO) 13.28 (s, 1H), 8.11-8.06 (m, 2H), 7.82 (d, 1H), 7.68 (d, 1H), | 496.1 |
| | 7.04 (d, 1H), 6.89 (s, 1H), 3.68-3.60 (m, 4H), 3.10-3.00 (m, 4H), 2.23 | (M + 1) |
| | (s, 3H) | |
| 3064 | (DMSO) 13.28 (s, 1H), 8.16 (d, 1H), 8.11 (d, 1H), 7.82 (d, 1H), 7.71- | 482.1 |
| | 7.67 (m, 2H), 7.04 (d, 1H), 6.85 (d, 1H), 3.64-3.60 (m, 4H), 3.10- | (M + 1) |
| | 3.00 (m, 4H) | |
| 3065 | (DMSO) 13.30 (s, 1H), 8.11 (d, 1H), 7.83 (d, 1H), 7.78 (t, 1H), 7.69 | 472.1 |
| | (d, 1H), 7.13-6.97 (m, 3H), 3.77-3.62 (m, 4H), 3.15-3.00 (m, 4H) | (M + 1) |
| 3068 | (DMSO) 13.30 (s, 1H), 8.55 (d, 1H), 8.21 (d, 1H), 8.13 (d, 1H), 7.85 | 506.1 |
| | (d, 1H), 7.71 (d, 1H), 7.05 (d, 1H), 3.60-3.50 (m, 4H), 3.20-3.10 (m, | (M + 1) |
| | 4H) | |
| 3069 | (DMSO) 13.24 (s, 1H), 8.25 (d, 1H), 8.10 (d, 1H), 7.82 (d, 1H), 7.79 | 530.2 |
| | (dd, 1H), 7.68 (d, 1H), 7.04 (dd, 1H), 6.73 (d, 1H), 3.67-3.57 (m, 4H), | (M + 1) |
| | 3.10-3.00 (m, 4H) | |
| 3070 | (DMSO) 13.23 (s, 1H), 8.11-8.07 (m, 2H), 7.82 (d, 1H), 7.68 (d, 1H), | 438.3 |
| | 7.62 (dd, 1H), 7.04 (d, 1H), 6.89 (d, 1H), 3.70-3.60 (m, 4H), 3.10- | (M + 1) |
| | 3.00 (m, 4H) | |
| 3071 | (DMSO) 13.29 (s, 1H), 8.11 (d, 1H), 7.82 (d, 1H), 7.68 (d, 1H), 7.45 | 418.4 |
| | (t, 1H), 7.04 (d, 1H), 6.63 (d, 1H), 6.54 (d, 1H), 3.65-3.55 (m, 4H), | (M + 1) |
| | 3.10-3.00 (m, 4H), 2.28 (s, 3H) | |
| 3105 | (DMSO): 13.30 (b s, 1H), 8.11-8.08 (m, 2H), 7.82 (d, 1H), 7.68 (d, | 404.0 |
| | 1H), 7.53 (m, 1H), 7.03 (d, 1H), 6.83 (d, 1H), 6.66 (dd, 1H), 3.63 (m, | (M + 1) |
| | 4H), 3.06 (m, 4H) | |
| 3256 | (CDCl3) 7.46 (d, 1H), 7.40 (t, 1H), 7.32 (d, 1H), 6.53 (d, 1H), 6.42 | 395.0 |
| | (d, 1H), 3.69 (t, 4H), 3.23-3.10 (m, 10H), 2.37 (s, 3H) | (M + 1) |
| 3264 | (CDCl3) 8.36 (dd, 1H), 7.81 (dd, 1H), 7.48 (d, 1H), 7.34 (d, 1H), 6.86 | 406.0 |
| | (q, 1H), 3.83 (t, 4H), 3.28 (t, 4H), 3.20-3.10 (m, 6H) | (M + 1) |
| 3265 | (CDCl3) 7.63 (t, 1H), 7.47 (d, 1H), 7.32 (d, 1H), 6.99 (d, 1H), 6.78 | 449.2 |
| | (d, 1H), 3.77 (t, 4H), 3.30-3.10 (m, 10H) | (M + 1) |
| 3286 | (DMSO) 8.96 (d, 1H), 8.26 (dd, 1H), 7.40-7.30 (m, 2H), 6.98 (d, 1H), | 383.0 |
| | 4.00-3.80 (m, 4H), 3.30-3.20 (m, 4H) | (M + 1) |
| 3292 | (DMSO) 8.43 (dd, 1H), 8.12 (dd, 1H), 7.39-7.36 (m, 2H), 7.01 (q, | 363.0 |
| | 1H), 3.70-3.60 (m, 4H), 3.50-3.00 (m, 4H) | (M + 1) |
| 3293 | (DMSO) 7.79 (t, 1H), 7.37-7.34 (m, 2H), 7.15 (d, 1H), 7.09 (d, 1H), | 406.1 |
| | 3.70-3.60 (m, 4H), 3.25-3.20 (m, 4H) | (M + 1) |
| 3302 | (DMSO): 13.14 (br s, 1H), 8.29 (s, 1H), 8.02 (s, 1H), 7.65 (m, 2H), | 418.2 |
| | 7.31 (m, 1H), 7.22 (m, 1H), 6.89 (s, 1H), 3.35 (m, 4H), 3.12 (m, 4H), | (M + 1) |
| | 2.40 (s, 3H) | |
| 3304 | (CDCl3) 7.42 (t, 1H), 7.07 (m, 2H), 6.56 (d, 1H), 6.43 (d, 1H), 3.67 | 379.1 |
| | (t, 4H), 3.34 (t, 4H), 3.26 (s, 3H), 3.10 (s, 3H), 2.38 (s, 3H) | (M + 1) |
| 3305 | (CDCl3) 7.64 (t, 1H), 7.08 (m, 2H), 7.02 (d, 1H), 6.79 (d, 1H), 3.76 | 433.1 |
| | (t, 4H), 3.35 (t, 4H), 3.26 (s, 3H), 3.10 (s, 3H) | (M + 1) |
| 3318 | (DMSO) 13.30 (s, 1H), 8.55 (s, 1H), 8.35 (dd, 1H), 8.13 (dd, 1H), | 429.1 |
| | 7.86 (dd, 1H), 7.74 (dd, 1H), 7.70 (d, 1H), 7.05 (d, 1H), 3.50-3.30 | (M + 1) |
| | (m, 4H), 3.20-3.00 (m, 4H) | |
| 3322 | (DMSO) 13.30 (s, 1H), 8.23 (d, 1H), 8.15 (d, 1H), 8.10 (d, 1H), 7.82 | 449.1 |
| | (d, 1H), 7.70 (d, 1H), 7.49 (dd, 1H), 7.04 (d, 1H), 3.70-3.60 (m, 4H), | (M + 1) |
| | 3.20-3.00 (m, 4H) | |
| 3328 | (CDCl₃) 9.02 (d, 1H), 8.26 (dd, 1H), 7.08 (d, 1H), 7.02 (d, 1H), 6.60 | 410.0 |
| | (d, 1H), 3.91 (t, 4H), 3.37 (t, 4H), 3.25 (s, 3H), 3.09 (s, 3H) | (M + 1) |
| 3329 | (CDCl3) 8.37 (dd, 1H), 7.82 (dd, 1H), 7.10-7.06 (m, 2H), 6.87 (dd, | 390.1 |
| | 1H), 3.80 (t, 4H), 3.40 (t, 4H), 3.28 (s, 3H), 3.11 (s, 3H) | (M + 1) |
| 3341 | (DMSO) 13.30 (s, 1H), 8.60 (s, 1H), 8.08 (d, 1H), 7.94 (dd, 1H), 7.79 | 476.1 |
| | (d, 1H), 7.67 (d, 1H), 7.02 (d, 1H), 6.88 (d, 1H), 4.24 (q, 2H), 3.85- | (M + 1) |
| | 3.70 (m, 4H), 3.10-3.00 (m, 4H), 1.28 (t, 3H) | |
| 3342 | (DMSO) 13.30 (s, 1H), 8.61 (d, 1H), 8.07 (d, 1H), 7.96 (dd, 1H), 7.79 | 462.1 |
| | (d, 1H), 7.67 (d, 1H), 7.02 (d, 1H), 6.88 (d, 1H), 3.83-3.77 (m, 4H), | (M + 1) |
| | 3.76 (s, 3H), 3.10-3.00 (m, 4H) | |
| 3355 | (DMSO) 13.30 (bs, 1H), 8.58 (d, 1H), 8.08 (d, 1H), 7.94 (dd, 1H), | 448.0 |
| | 7.79 (d, 1H), 7.67 (d, 1H), 7.02 (d, 1H), 6.88 (d, 1H), 3.83-3.75 (m, | (M + 1) |
| | 4H), 3.10-3.05 (m, 4H) | |
| 3359 | (DMSO) 8.91 (s, 1H), 8.22 (d, 1H), 7.87 (s, 1H), 7.80 (d, 1H), 7.59 | 448.1 |
| | (d, 1H), 7.45 (s, 1H), 6.93 (d, 1H), 6.44 (d, 1H), 4.00-3.90 (m, 4H), | (M + 1) |
| | 3.10-3.00 (m, 4H) | |
| 3360 | (DMSO) 13.30 (s, 1H), 8.08 (d, 1H), 7.92 (d, 1H), 7.79 (d, 1H), 7.66 | 418.2 |
| | (d, 1H), 7.38 (dd, 1H), 7.03 (d, 1H), 6.76 (d, 1H), 3.60-3.50 (m, 4H), | (M + 1) |
| | 3.10-3.00 (m, 4H), 2.10 (s, 3H) | |
| 3361 | (DMSO) 8.40 (dd, 1H), 8.26 (dd, 1H), 8.09 (d, 1H), 7.81 (d, 1H), 7.68 | 449.2 |
| | (d, 1H), 7.03 (d, 1H), 6.97 (dd, 1H), 3.70-3.20 (m, 4H), 3.20-3.00 (m, | (M + 1) |
| | 4H) | |
| 3362 | (DMSO) 13.28 (br s, 1H), 8.09 (d, 1H), 7.82 (d, 1H), 7.68 (d, 1H), | 410.2 |
| | 7.16 (d, 1H), 7.02 (d, 1H), 6.88 (d, 1H), 3.50 (m, 4H), 3.13 (m, 4H) | (M + 1) |
| 3365 | (DMSO) 13.28 (br s, 1H), 8.38 (s, 1H), 8.06 (d, 1H), 7.79-7.78 (m, | 472.1 |
| | 2H), 7.66 (d, 1H), 7.01 (d, 1H), 6.94 (d, 1H), 3.76 (m, 4H), 3.08 (m, | (M + 1) |
| | 4H) | |
| 3422 | (DMSO): 13.33 (br s, 1H), 10.18 (br s, 1H), 8.06 (d, 1H), 7.95 (d, | 637.3 |
| | 1H), 7.91 (d, 1H), 7.79 (d, 1H), 7.67 (d, 1H), 7.36 (dd, 1H), 7.02 (d, | (M + 1) |
| | 1H), 3.65 (m, 2H), 3.50-3.35 (m, 12H), 3.23 (m, 4H), 3.19 (s, 3H), | |
| | 3.12 (m, 4H), 2.55 (t, 2H) | |
| 3439 | (DMSO): 13.30 (br, 1H), 8.52 (s, 1H), 8.06 (d, 1H), 7.95 (dd, 1H), | 594.1 |
| | 7.79 (d, 1H), 7.67 (d, 1H), 7.04 (d, 1H), 6.88 (d, 1H), 4.31 (t, 2H), | (M + 1) |
| | 3.82 (m, 4H), 3.70 (t, 2H), 3.56-3.35 (m, 8H), 3.21 (s, 3H), 3.09 (m, | |
| | 4H) | |
| 3440 | (DMSO): 13.29 (br s, 1H), 10.24 (br s, 1H), 8.11 (d, 1H), 7.97 (d, | 461.2 |
| | 1H), 7.91 (d, 1H), 7.84 (d, 1H), 7.70 (d, 1H), 7.40 (dd, 1H), 7.00 (d, | (M + 1) |
| | 1H), 3.23 (m, 4H), 3.13 (m, 4H), 2.03 (s, 3H) | |
| 3454 | (MeOD:DMSO:TFA-d, 10:1:0.05): 8.04-8.01 (m, 2H), 7.92 (d, 1H), | 518.2 |
| | 7.70 (d, 1H), 7.63 (d, 1H), 7.58 (dd, 1H), 7.05 (d, 1H), 3.88 (d, 1H), | (M + 1) |
| | 3.34 (m, 4H), 3.24 (m, 4H), 2.26 (sep, 1H), 1.08 (d, 3H), 1.05 (d, 3H) | |
| 3455 | (DMSO) 13.30 (s, 1H), 8.12 (d, 1H), 7.93 (s, 1H), 7.83 (d, 1H), 7.70 | 419.1 |
| | (d, 1H), 7.53 (dd, 1H), 7.05-6.94 (m, 2H), 3.64-3.55 (m, 4H), 3.15- | (M + 1) |
| | 3.00 (m, 4H) | |
| 3477A | (DMSO): 13.01 (br s, 1H), 8.12 (d, 1H), 7.88-7.84 (m, 2H), 7.71 (d, | 419.2 |
| | 1H), 7.63 (br s, 3H), 7.37 (d, 1H), 7.05 (d, 1H), 6.95 (d, 1H), 3.13 (m, | (M + 1) |
| | 8H) | |
| 3481A | (DMSO): 13.31 (br s, 1H), 10.83 (s, 1H), 8.27 (br s, 3H), 8.12 (d, | 566.2 |
| | 1H), 8.02 (d, 1H), 7.88 (d, 1H), 7.85 (d, 1H), 7.71 (d, 1H), 7.44 (dd, | (M + 1) |
| | 1H), 7.31-7.25 (m, 5H), 7.04 (d, 1H), 4.22 (m, 1H), 3.27 (br m, 4H), | |
| | 3.13 (br m, 4H), 3.02 (m, 2H) | |
| 3482 | (DMSO): 13.28 (br s, 1H), 8.58 (d, 1H), 8.08 (d, 1H), 7.95 (dd, 1H), | 447.0 |
| | 7.81 (d, 1H), 7.75 (br s, 1H), 7.68 (d, 1H), 7.12 (br s, 1H), 7.02 (d, | (M + 1) |
| | 1H), 6.84 (d, 1H), 3.76 (m, 4H), 3.07 (m, 4H) | |
| 3486A | (DMSO): 13.31 (br s, 1H), 10.94 (s, 1H), 8.25 (br s, 3H), 8.11 (d, | 532.2 |
| | 1H), 8.05 (d, 1H), 7.91 (d, 1H), 7.84 (d, 1H), 7.71 (d, 1H), 7.45 (dd, | (M + 1) |
| | 1H), 7.03 (d, 1H), 3.99 (m, 1H), 3.29 (br m, 4H), 3.14 (br m, 4H), | |
| | 1.63 (br m, 3H), 0.89 (br m, 6H) | |
| 3487A | (DMSO): 13.31 (br s, 1H), 10.93 (s, 1H), 9.79 (br s, 1H), 8.11 (d, | 504.1 |
| | 1H), 8.04 (d, 1H), 7.89 (d, 1H), 7.84 (d, 1H), 7.71 (d, 1H), 7.45 (dd, | (M + 1) |
| | 1H), 7.04 (d, 1H), 4.12 (s, 2H), 3.28 (br m, 4H), 3.13 (br m, 4H), 2.85 | |
| | (m, 6H) | |
| 3503 | (DMSO): 13.23 (br s, 1H), 8.10 (d, 1H), 7.82 (d, 1H), 7.65 (d, 1H), | 447.2 |
| | 7.28-7.22 (m, 2H), 7.03 (d, 1H), 6.92-6.88 (m, 3H), 4.02 (t, 2H), 2.99 | (M + 1) |
| | (m, 4H), 2.72 (m, 2H), 2.61 (m, 4H) | |
| 3828 | (DMSO) 8.56 (s, 1H), 8.35 (d, 1H), 8.15 (d, 1H), 7.88 (d, 1H), 7.74- | 443.2 |
| | 7.70 (m, 2H), 7.11 (d, 1H), 3.70 (s, 3H), 3.48-3.33 (m, 4H), 3.20-3.18 | (M + 1) |
| | (m, 4H) | |
| 3846 | (DMSO) 13.28 (s, 1H), 8.32 (s, 1H), 8.07 (s, 1H), 7.85 (d, 1H), 7.64 | 443.2 |
| | (dd, 2H), 6.87 (s, 1H), 3.71 (t, 4H), 3.10 (t, 4H), 2.41 (s, 3H) | (M + 1) |
| 3847 | (DMSO) 13.28 (s, 1H), 8.56 (s, 1H), 8.34 (d, 1H), 7.70 (d, 2H), 7.65 | 419.2 |
| | (d, 1H), 6.89 (s, 1H), 3.42 (t, 4H), 3.19 (t, 4H), 2.41 (s, 3H) | (M + 1) |
| 3849 | (DMSO) 13.30 (s, 1H), 8.11 (d, 1H), 7.84 (d, 1H), 7.69(d, 1H), 7.04 | 409.2 |
| | (d, 1H), 6.75 (m, 2H), 6.19 (m, 1H), 3.20 (d, 8H) | (M + 1) |
| 3881 | (DMSO) 13.29 (s, 1H), 8.10 (d, 1H), 7.82 (d, 1H), 7.69 (d, 1H), 7.40 | 444.1 |
| | (d, 1H), 7.30 (d, 1H), 7.17 (t, 1H), 7.05-7.00 (m, 2H), 3.80-3.70 (m, | (M + 1) |
| | 4H), 3.30-3.10 (m, 4H) | |
| 3882 | (DMSO) 13.30 (s, 1H), 8.13-8.03 (m, 3H), 7.86 (d, 1H), 7.71 (d, 1H), | 460.0 |
| | 7.57-7.52 (m, 1H), 7.44-7.37 (m, 1H), 7.05 (d, 1H), 3.70-3.50 (m, | (M + 1) |
| | 4H), 3.50-3.20 (m, 4H) | |
| 3884 | (DMSO) 13.28 (s, 1H), 8.44 (s, 2H), 8.09 (d, 1H), 7.80 (d, 1H), 7.66 | 423.1 |
| | (d, 1H), 7.03 (d, 1H), 3.90-3.80 (m, 4H), 3.20-3.00 (m, 4H) | (M + 1) |
| 3885 | (DMSO) 13.28 (s, 1H), 8.35 (d, 2H), 8.09 (d, 1H), 7.80 (d, 1H), 7.66 | 405.1 |
| | (d, 1H), 7.03 (d, 1H), 6.66-6.63 (m, 1H), 4.00-3.80 (m, 4H), 3.20- | (M + 1) |
| | 3.00 (m, 4H) | |
| 3907 | (DMSO) 13.30 (s, 1H), 8.26 (d, 1H), 8.12 (d, 1H), 8.02 (d, 1H), 7.85 | 439.1 |
| | (d, 1H), 7.70 (d, 1H), 7.05 (d, 1H), 3.52-3.45 (m, 4H), 3.26-3.10 (m, | (M + 1) |
| | 4H) | |
| 3925 | (DMSO) 13.29 (s, 1H), 8.32 (d, 1H), 8.13 (d, 1H), 7.87 (d, 1H), 7.74- | 429.2 |
| | 7.61 (m, 3H), 7.05 (d, 1H), 3.50-3.30 (m, 4H), 3.30-3.10 (m, 4H) | (M + 1) |
| 3927 | (DMSO) 13.30 (s, 1H), 8.64 (s, 1H), 8.51 (s, 1H), 8.12 (d, 1H), 7.84 | 507.1 |
| | (s, 1H), 7.69 (s, 1H), 7.05 (d, 1H), 3.70-3.60 (m, 4H), 3.20-3.00 (m, | (M + 1) |
| | 4H) | |
| 3937 | (DMSO) 13.26 (s, 1H), 8.49 (s, 1H), 8.28 (d, 1H), 8.10 (d, 1H), 7.74 | 443.2 |
| | (m, 2H), 7.67 (d, 1H), 7.03 (dd, 1H), 4.21 (m, 1H), 3.76 (d, 1H), 3.54 | (M + 1) |
| | (d, 1H), 3.43 (m, 2H), 3.07 (m, 2H), 1.22 (d, 3H) | |
| 3976 | (DMSO): 13.28 (s, 1H), 8.31(s, 1H), 8.07 (d, 1H), 7.83 (dd, 2H), 7.68 | 405.1 |
| | (d, 2H), 7.02 (dd, 1H), 3.66 (m, 4H), 3.11 (m, 4H) | (M + 1) |
| 4039 | (DMSO) 13.29 (s, 1H), 8.71 (d, 1H), 8.58 (d, 1H), 8.11 (d, 1H), 7.83 | 454.0 |
| | (d, 1H), 7.69 (d, 1H), 7.04 (d, 1H), 4.10-3.90 (m, 4H), 3.20-3.00 (m, | (M + 1) |
| | 4H) | |
| 4054 | (DMSO) 13.29 (s, 1H), 8.43 (d, 1H), 8.14 (d, 1H), 8.09 (d, 1H), 7.83 | 430.1 |
| | (d, 1H), 7.70 (d, 1H), 7.03 (d, 1H), 3.84 (t, 4H), 3.17 (t, 4H) | (M + 1) |
| 4055 | (DMSO) 13.30 (s, 1H), 8.10 (m, 3H), 7.85 (d, 1H), 7.70 (d, 1H), 7.04 | 419.2 |
| | (d, 1H), 3.27 (d, 4H), 3.18 (d, 4H), 2.39 (s, 3H) | (M + 1) |
| 4072 | (DMSO) 13.29 (s, 1H), 8.21 (d, 1H), 8.10 (d, 1H), 7.99 (s, 1H), 7.96 | 448.2 |
| | (d, 1H), 7.83 (d, 1H), 7.68 (d, 1H), 7.56 (s, 1H), 7.03 (dd, 1H), 3.54 | (M + 1) |
| | (t, 4H), 3.11 (t, 4H) | |
| 4074 | (DMSO) 8.56 (s, 1H), 8.34 (d, 1H), 7.74 (d, 1H), 7.57 (d, 1H), 7.45(d, | 396.3 |
| | 1H), 3.83 (t, 4H), 3.75 (t, 2H), 3.49 (t, 2H), 3.39 (t, 4H), 1.90 (m, 4H) | (M + 1) |
| 4075 | (DMSO) 8.43 (dd, 1H), 8.10 (dd, 1H), 7.56 (d, 1H), 7.45 (d, 1H), 6.96 | 396.2 |
| | (dd, 1H), 3.80 (m, 4H), 3.73 (m, 6H), 3.49 (t, 2H), 1.89 (m, 4H) | (M + 1) |
| 4096 | (DMSO) 8.56 (s, 1H), 8.51 (s, 1H), 8.35 (d, 1H), 7.71 (d, 1H), 7.64 | 420.3 |
| | (d, 1H), 7.31 (d, 1H), 5.32 (m, 1H), 4.69 (t, 1H), 4.18 (dd, 1H), 3.41 | (M + 1) |
| | (s, 4H), 3.15 (s, 4H) | |
| 4097 | (DMSO) 8.48 (s, 1H), 8.42 (dd, 1H), 8.10 (dd, 1H), 7.61 (d, 1H), 7.28 | 420.3 |
| | (d, 1H), 6.99 (dd, 1H), 5.30 (m, 1H), 4.67 (t, 1H), 4.16 (dd, 1H), | (M + 1) |
| | 3.41(t, 4H), 3.15 (t, 4H) | |
| 4098 | (DMSO) 13.30 (s, 1H), 8.21 (dd, 1H), 8.10 (d, 1H), 7.84 (d, 1H), 7.79 | 447.1 |
| | (s, 1H), 7.71 (m, 2H), 7.46 (s, 1H), 7.04 (d, 1H), 6.92 (m, 1H), 3.37 | (M + 1) |
| | (t, 4H), 3.14 (t, 4H) | |
| 4144 | (DMSO): 13.28 (s, 1H), 8.19 (m, 1H), 8.12 (d, 1H), 7.84 (d, 1H), 7.70 | 475.2 |
| | (d, 1H), 7.46 (d, 1H), 7.03 (d, 1H), 6.89 (m, 1H), 3.38 (brs, 4H), 3.17 | (M + 1) |
| | (brs, 4H), 2.92 (s, 3H), 2.70 (s, 3H) | |
| 4184 | (MeOD-d4) 8.20 (dd, 1H), 8.04 (d, 1H), 7.96 (dd, 1H), 7.68 (d, 1H), | 461.3 |
| | 7.62 (d, 1H), 7.08-7.04 (m, 2H), 3.55-3.47 (m, 4H), 3.27-3.23 (m, | (M + 1) |
| | 4H), 2.86 (s, 3H) | |
| 4275 | (DMSO) 13.29 (s, 1H), 8.26 (d, 2H), 7.63 (q, 2H), 7.17 (d, 2H), 6.89 | 418.2 |
| | (s, 1H), 3.83 (t, 4H), 3.18 (t, 4H), 2.38 (d, 3H) | (M + 1) |
| 4501 | (DMSO): 13.30 (s, 1H), 8.24 (d, 2H), 8.13 (d, 1H), 7.87 (d, 1H), 7.71 | 418.3 |
| | (d, 1H), 7.05 (d, 1H), 6.91 (d, 1H), 3.16 (d, 4H), 3.09 (d, 4H), 2.73 | (M + 1) |
| | (s, 3H) | |
| 4502 | (DMSO) 13.30 (s, 1H), 8.46 (s, 2H), 8.12 (d, 1H), 7.85 (d, 1H), 7.73 | 472.4 |
| | (d, 1H), 7.04 (d, 1H), 3.38 (d, 4H), 3.16 (s, 4H) | (M + 1) |
| 4503 | (DMSO) 13.30 (s, 1H), 8.13 (d, 1H), 7.86 (d, 1H), 7.69 (d, 1H), 7.52 | 419.2 |
| | (d, 1H), 7.05 (d, 1H), 6.93 (d, 1H), 6.79 (m, 1H), 4.86 (s, 2H), 3.19 | (M + 1) |
| | (d, 4H), 3.09 (d, 4H) | |
| 4829 | (DMSO) 13.30 (s, 1H), 8.56 (s, 1H), 8.35 (d, 1H), 7.72 (s, 1H), 7.70 | 457.4 |
| | (s, 1H), 7.65-7.60 (m, 1H), 6.81 (s, 1H), 3.42 (brs, 4H), 3.18 (brs, | (M + 1) |
| | 4H), 2.77-2.70 (q, 2H), 1.15-1.10 (t, 3H) | |
| 4830 | (DMSO) 13.31 (s, 1H), 8.41 (s, 1H), 8.18 (d, 1H), 7.91-7.88 (m, 1H), | 432.4 |
| | 7.71-7.67 (m, 2H), 7.64-7.58 (m, 1H), 6.81 (s, 1H), 3.51 (brs, 4H), | (M + 1) |
| | 3.15 (brs, 4H), 2.71-2.68 (q, 2H), 1.14-1.09 (t, 3H) | |
| 4831 | (DMSO) 13.31 (s, 1H), 8.09 (s, 2H), 7.70-7.65 (m, 1H), 7.63-7.59 | 447.4 |
| | (m, 1H), 6.81 (s, 1H), 3.28 (brs, 4H), 3.17 (brs, 4H), 2.77-2.69 (q, | (M + 1) |
| | 2H), 2.40 (s, 3H), 1.15-1.10 (t, 3H) | |
| 4874 | (DMSO) 13.29 (s, 1H), 8.32 (s, 1H), 8.08 (s, 1H), 7.86 (d, 1H), 7.66- | 433.4 |
| | 7.57 (m, 2H), 6.79 (s, 1H), 3.72 (brs, 4H), 3.10 (brs, 4H), 2.74-2.67 | (M + 1) |
| | (q, 2H), 1.12-1.07 (t, 3H) | |
| 5068 | (DMSO) 13.32 (s, 1H), 8.78 (s, 1H), 8.32 (d, 1H), 8.10 (d, 1H), 7.85 | 405.3 |
| | (d, 1H), 7.70 (d, 1H), 7.14 (d, 1H), 7.02 (d, 1H), 3.98 (brs, 4H), 3.16 | (M + 1) |
| | (brs, 4H) | |
| 5071 | (DMSO) 13.30 (s, 1H), 8.56 (s, 1H), 8.42 (s, 1H), 8.10 (d, 1H), 7.83 | 439.3 |
| | (d, 1H), 7.68 (d, 1H), 7.03 (d, 1H), 3.76 (brs, 4H), 3.12 (brs, 4H) | (M + 1) |
| 5072 | (DMSO) 13.29 (s, 1H), 8.49 (s, 1H), 8.17 (s, 1H), 8.10 (d, 1H), 7.83 | 419.3 |
| | (d, 1H), 7.67 (d, 1H), 7.05 (m, 1H), 3.53 (brs, 4H), 3.10 (brs, 4H), | (M + 1) |
| | 2.13 (s, 3H) | |
| 5073 | (DMSO) 13.30 (s, 1H), 8.26 (brs, 1H), 8.10 (d, 2H), 7.84 (d, 1H), | 422.4 |
| | 7.69 (d, 1H), 6.99 (m, 2H), 3.37 (brs, 4H), 3.12 (brs, 4H) | (M + 1) |

### Example 3. Potency of Inhibitor Compounds

i. Transport Inhibition Assays.

Transport experiments were conducted to examine the affinity of the inhibitor compounds for PSAC. Inhibitor affinity for blocking PSAC was determined using a quantitative transmittance assay which is based on the osmotic lysis of infected cells with sorbitol (Desai, S., and A. Pillai, U.S. Pat. No. 8,618,090, supra (2013); Dondorp et al., supra (2009); Hooft van Huijsduijnen, R., and T. N. Wells, supra (2018)). Malaria parasite cultures were enriched at the trophozoite stage via the Percoll-sorbitol method (Pillai et al., Mol. Pharmacol., 82:1104-1114 (2012); Pillai et al., Mol. Pharmacol., 77:724-733 (2010); Wagner et al., Biophys. J., 84:116-123 (2003)), washed, and resuspended at 37° C. and 0.15% hematocrit in 280 mM sorbitol, 20 mM Na-HEPES, 0.1 mg/ml bovine serum albumin, pH 7.4, with the indicated concentrations of inhibitor compounds. Osmotic lysis, resulting from PSAC-mediated sorbitol uptake, was continuously tracked with transmittance measurements through the cell suspension (700-nm wavelength, DU640 spectrophotometer with Peltier temperature control; Beckman Coulter, Fullerton, CA).

Inhibitor dose-response relationships (K_{0.5} values) were calculated from the times required to reach a fractional lysis threshold. K_{0.5} estimates obtained with the osmotic lysis assay match those obtained using cell-attached and whole-cell patch-clamp assays (Alkhalil et al., Blood, 104:4279-4286 (2004); Dondorp et al., N. Engl. J. Med., 361:455-467 (2009)), confirming a quantitatively robust and valid assay. The results are shown in Table 6 (PSAC K_{0.5}).

ii. In vitro Parasite Growth Inhibition Studies.

P. falciparum laboratory lines were propagated at the asexual stage with standard methods, in RPMI 1640 medium supplemented with 25 mM HEPES, 31 mM NaHCO3, 0.37 mM hypoxanthine, 10 μg/ml gentamicin, and 10% pooled human serum. Nutrient deprivation experiments utilized standard PSAC Growth Inhibition Medium (PGIM) but with reduced concentrations of individual ingredients. Human serum was exhaustively dialyzed against distilled water before addition to those media. The PGIM contained reduced concentrations of isoleucine (11.4 μM), glutamine (102 μM), and hypoxanthine (3.01 μM) and was supplemented with the dialyzed human serum. Growth inhibition experiments (Burrows et al., Parasitology, 141:128-139 (2013)) were quantified by using a SYBR Green I-based fluorescence assay for parasite nucleic acid in 96-well microplates, as described previously (Dondorp et al., supra (2009)). Ring-stage synchronized cultures were seeded at 1% parasitemia and 2% hematocrit levels in standard medium or PGIM and were maintained for 72 hr at 37° C. in 5% O₂/5% CO₂ in nitrogen, without medium changes. Cultures were then lysed in 20 mM Tris, 10 mM EDTA, 0.016% saponin, 1.6% Triton X-100, pH 7.5, with SYBR Green I nucleic acid gel stain (Invitrogen, Carlsbad, CA) at 5000-fold dilution.

After a 45-mM incubation, parasite DNA contents were quantified through fluorescence measurements (excitation, 485 nm; emission, 528 nm). Results were presented as IC₅₀ values, defined as the concentration of inhibitor which decreases P. falciparum (Pf) DNA content by 50% (IC₅₀ PGIM). The results are shown in Table 7.

### Example 4. In vitro ADMET Methods

i. Mammalian Cytotoxicity.

Cytotoxicity of the inhibitor compounds was quantified using human HeLa cells (CLL-2; American Type Culture Collection, Manassas, VA) seeded at 4000 cells/well in 96-well plates. Cultures were incubated with individual inhibitor compounds for 72 hr at 37° C. in minimal essential medium (Invitrogen) supplemented with 10% fetal calf serum. Cell viability was quantified using the vital stain 3-(4,5-dimethylthiazol-2-yl)-5-(3-carboxymethonyphenol)-2-(4-sulfophenyl)-2H-tetrazolium, inner salt (MTS) (5).

Mammalian cell toxicity (CC₅₀) is defined as the concentration of inhibitor which decreases cell viability by 50% (HeLa CC₅₀). The results are shown in Table 6.

ii. Microsome and Serum Stability Assays.

The microsome and serum stability assays measure the stability of the inhibitor compounds when exposed to the liver and to serum in vivo.

For microsomal stability measurements, inhibitor compounds were incubated with mouse microsomal proteins in buffer with NADPH (Bevan, C. and R. Lloyd, Anal. Chem., 72:1781-1787 (2000)) and the resulting samples were analyzed by LC/MS to quantitate the remaining parent. The data are calculated as percent of parent remaining. The results are shown in Table 7 (MLMS).

For serum stability, inhibitor compounds were exposed to 55% mouse serum and the resulting samples analyzed by LC/MS to quantitate the remaining parent. The data are calculated as percent of parent remaining. The results are shown in Table 7 (MSS).

iii. Cytochrome P450 Inhibition.

To measure Cytochrome P450 inhibition, a fluorogenic substrate assay was used to measure inhibitor compound interference with enzymes that metabolize drugs in vivo (Houston, J., Biochem. Pharmacol., 47:1469-1479 (1994)). The percent inhibition of the inhibitor compounds is set forth in Table 7 (Cyp 3A4).

iv. Caco-2 Permeability.

The Caco-2 assay is an indicator of the potential oral bioavailability of the inhibitor compound. Caco-2 cells were applied to wells of a collagen-coated BioCoat Cell Environment (BD Biosciences) plate as described (Duraisingh, M., and A. Cowman, Acta Trop., 94:181-190 (2005)). The test agent was added to the apical (A) side and the amount of permeation was determined on the basolateral (B) side by LC/MS/MS analysis. The amount of material on the A and B sides were used to calculate a Pₐₚₚ value (Caco-2 Pₐₚₚ). The results are shown in Table 7.

v. Solubility.

The solubility of the inhibitor compounds was determined by the nephelometric method (Bevan, C., and R. Lloyd, supra (2000)). A high-throughput screening method for the determination of aqueous drug solubility using laser nephelometry in microtiter plates.

The compounds were serially diluted in DMSO and then added to water. The results show the highest concentration of compound that does not cause a sharp increase in the slope of the relative absorption/concentration curve. Additionally, to confirm the nephelometry results, compounds were stirred with a test vehicle (deionized water, PBS, or D5W) at 20° C. for 4 hours. Additional compound was added if complete dissolution was obtained. The mixture was then filtered and the filtrate assayed by HPLC to quantify the amount of compound in solution (Solubility). The results are shown in Table 7.

### Example 5. Murine Studies

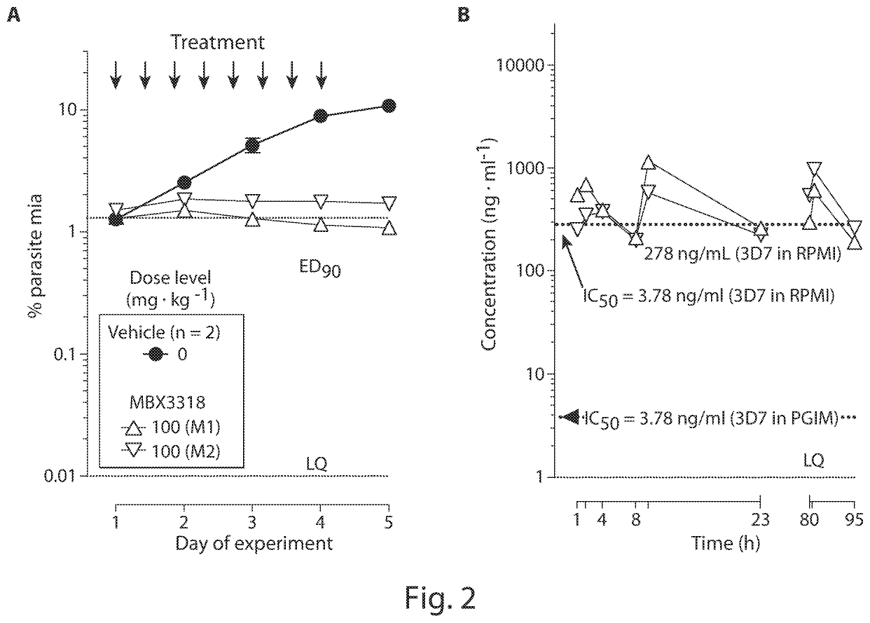

Tolerability. The test compounds were dissolved at concentrations of 1 to 10 mg/mL in various formulations (5% DMSO, 5% cremophor, 80% PEG-300 or 10% DMSO, 80% PEG-400, 1% Polysorbate 80). Male CD-1 mice were dosed with 4 dose levels of each test compound (vehicle control plus 3 dose levels ranging from 10-200 mg/kg as described in Table 8) of test compound by the intravenous (IV), intraperitoneal (IP) or oral (PO) route. Clinical observations described in Table 8 were made at various time points out to 24 hours after which mice were humanely euthanized.
Pharmacokinetics (PK). The test compounds were formulated as described above and male CD-1 mice were dosed with single dose levels of from 10-40 mg/kg (Table 9) of test compounds by the IV and PO routes. Serial blood samples were collected at 7 time points (0.083, 0.25, 0.5, 1, 4, 8, 24 hrs) per route. Samples were processed and compound content analyzed by LC/MS. PK parameters were calculated using the WinNonLin program.
Efficacy. A humanized mouse model was used for testing compounds MBX 3318 and 4055. Briefly, NOD-scid IL-2Rγ null (NSG) mice engrafted with human erythrocytes (hEr) for 10 days were infected with P. falciparum Pf3D70087/N9. Mice were then treated with 100 mg/kg of MBX 3318 (an analog of ISG-21 with a heteroaryl in position R² of Formula I) twice daily for 4 days by the oral route. The results are shown in FIG. 2.

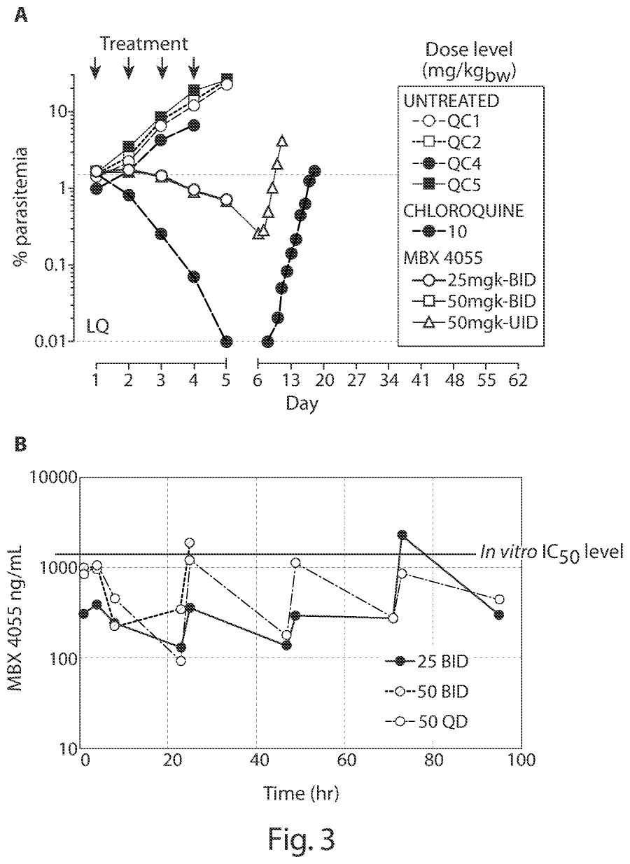

In a second study, NSG mice were treated with either 25 mg/kg or 50 mg/kg of MBX 4055 either once or twice daily for 4 days by the oral route. Control mice were treated with vehicle alone (10% DMSO, 80% PEG400, 1% Polysorbate 80 for MBX 3318 or 6.25% DMSO, 12.5% polysorbate 80, 43.75% Labrasol, 1.25% hydroxypropyl methylcellulose in H₂O for MBX 4055). Percent parasitemia was measured daily for 5 days as were the plasma levels of MBX 3318 and 4055. The results are shown in FIG. 3.

TABLE 6
In vitro PSAC and cytotoxicity data.

| Compound No. | PSAC K_{0.5}^{a} | HeLa CC₅₀^{b} | SI^{c} |
|---|---|---|---|
| MBX- | μM | μM | CC₅₀/PSAC K_{0.5} |
| ISG-21 (Sc I) | 0.003 | 86 | 28,700 |
| 2937 | 0.027 | >200 | >7,400 |
| 2997 | 0.007 | >200 | >28,500 |
| 3000 | 0.05 | >200 | >4,000 |
| 3037 | 0.019 | 10.1 | 532 |
| 3060 | 0.051 | >100 | >1,960 |
| 3061 | 0.008 | 61 | 7,625 |
| 3063 | 0.039 | >100 | >2,560 |
| 3064 | 0.044 | 79.5 | 1,807 |
| 3065 | 0.017 | 65.4 | 3,847 |
| 3068 | 0.033 | >100 | >3,000 |
| 3069 | 0.14 | >100 | 715 |
| 3070 | 0.068 | 80.5 | 1,184 |
| 3071 | 0.015 | >100 | >6,700 |
| 3105 | 0.024 | >200 | >8,330 |
| 3256 | 0.433 | >200 | >462 |
| 3264 | 0.886 | >200 | >225 |
| 3265 | 0.211 | >200 | >948 |
| 3286 | 1.58 | >200 | >127 |
| 3292 | 1.05 | >200 | >190 |
| 3293 | 1.38 | >200 | >145 |
| 3302 | 0.007 | >200 | >28,571 |
| 3304 | 2.22 | >200 | >90 |
| 3305 | 2.37 | 189 | 80 |
| 3318 | 0.016 | >200 | >12,500 |
| 3322 | 0.011 | 47.9 | 4,355 |
| 3328 | 2.1 | >200 | >95 |
| 3329 | 3 | >200 | >67 |
| 3341 | 0.006 | 183.8 | 30,633 |
| 3342 | 0.004 | 100 | 25,000 |
| 3355 | 0.169 | 100 | 592 |
| 3359 | 0.213 | >200 | >940 |
| 3360 | 0.03 | >200 | >6,667 |
| 3361 | 0.034 | >200 | >5,882 |
| 3362 | 0.055 | 131.3 | 2,387 |
| 3365 | 1.15 | >200 | >174 |
| 3383 | 0.14 | >200 | >1,429 |
| 3422 | 0.004 | >200 | >50,000 |
| 3439 | 0.003 | 193.7 | 64,567 |
| 3440 | 0.003 | >200 | >66,667 |
| 3454 | 0.008 | >200 | >25,000 |
| 3455 | 0.013 | >200 | >15,385 |
| 3477A | 0.027 | >200 | >7,407 |
| 3481A | 0.01 | >200 | >20,000 |
| 3482 | 0.026 | >200 | >7,692 |
| 3486A | 0.028 | >200 | >7,143 |
| 3487A | 0.023 | >200 | >8,696 |
| 3503 | 0.046 | >200 | >4,348 |
| 3828 | 5.1 | >200 | >40 |
| 3846 | 0.0073 | >200 | >27,397 |
| 3847 | 0.003 | >200 | >66,667 |
| 3849 | 0.265 | >200 | >755 |
| 3881 | 0.022 | 60.2 | 2,736 |
| 3882 | 0.08 | >200 | >2,500 |
| 3884 | 0.051 | 50.8 | 996 |
| 3885 | 0.053 | >200 | >3,774 |
| 3907 | 0.009 | >200 | >22,222 |
| 3925 | 0.027 | >200 | >7,407 |
| 3927 | 0.032 | 75.4 | 2,357 |
| 3937 | 0.158 | >200 | >1,266 |
| 3976 | 0.017 | >200 | >11,765 |
| 4039 | 0.039 | 12.2 | 313 |
| 4054 | 0.053 | >200 | >3,773 |
| 4055 | 0.021 | >200 | >9,524 |
| 4072 | 0.211 | >200 | >948 |
| 4074 | 2.2 | >200 | >91 |
| 4075 | 1.08 | >200 | >185 |
| 4096 | >>2 | 181.5 | N/A |
| 4097 | >>2 | >200 | N/A |
| 4098 | 0.44 | >200 | >455 |
| 4144 | 0.22 | >200 | >909 |
| 4184 | 0.62 | >200 | >323 |
| 4275 | 0.011 | >200 | >18,182 |
| 4501 | 0.01 | >200 | >20,000 |
| 4502 | 0.002 | >200 | >100,000 |
| 4503 | 0.094 | >200 | >2,128 |
| 4829 | 0.0089 | >200 | >22,472 |
| 4830 | 0.011 | 104.3 | 9,482 |
| 4831 | 0.014 | >200 | >14,286 |
| 4874 | 0.013 | >200 | >15,385 |
| 5068 | 0.024 | >200 | >8,333 |
| 5071 | 0.017 | >200 | >11,765 |
| 5072 | 0.015 | >200 | >13,333 |
| 5073 | 0.0075 | >200 | >26,667 |

^{a}Activity in the PSAC assay;
^{b}Cytotoxicity against HeLa cells (3 days);
^{c}Selectivity index as calculated by dividing cytotoxicity (CC₅₀) by growth inhibition (IC₅₀).

TABLE 7
In vitro ADME results

| | | Caco-2 | | | Mouse | Cyp 3A4^{f} | |
|---|---|---|---|---|---|---|---|
| Compound | IC₅₀ | Pₐₚₚ^{b} | MLMS^{c} | MSS^{d} | PB^{e} | % | |
| No. | PGIM^{a} | ×10⁻⁶ | % | % | % | inhib. | Solubility^{g} |
| MBX- | μM | cm/sec | consumed | consumed | bound | at 5 μM | μM |
| ISG-21 | 0.002 | 0.00 | 17 | 0 | 100 | 0 | 25 |
| (SC I) | | | | | | | |
| 2937 | 0.019 | 6.5 | 50 | 3 | — | 67 | 25 |
| 2997 | 0.038 | 0.00 | — | — | — | — | 25 |
| 3000 | 0.049 | 8.8 | — | — | — | 52 | 25 |
| 3061 | 0.016 | 9.8 | 0 | 0 | — | 13 | 25 |
| 3063 | 0.032 | 0.00 | — | — | — | — | 50 |
| 3064 | 0.126 | 0.00 | — | — | — | 6 | — |
| 3065 | 0.01 | 0.00 | — | — | — | — | 25 |
| 3068 | 0.015 | 0.00 | — | — | — | — | 25 |
| 3071 | 0.026 | 0.00 | — | — | — | — | 25 |
| 3105 | 0.03 | 0.0 | — | — | — | 11 | 25 |
| 3302 | 0.002 | 0.0 | — | — | — | 38 | 50 |
| 3318 | 0.023 | 11.2 | 37 | 0 | 89 | 37 | 25 |
| 3322 | 0.007 | 9.6 | 0 | — | — | 9 | 25 |
| 3341 | 0.016 | 0.00 | — | — | — | 6 | — |
| 3342 | 0.008 | 0.00 | 2 | — | — | 1 | 25 |
| 3422 | 0.003 | 0.00 | 38 | — | — | 12 | 25 |
| 3439 | <0.0004 | 0.00 | 100 | — | — | 16 | 25 |
| 3440 | 0.008 | 0.00 | 0 | — | — | 1 | 12.5 |
| 3454 | 0.003 | 0.00 | 0 | — | — | 49 | 50 |
| 3455 | 0.118 | 3.6 | 27 | — | — | 20 | >400 |
| 3477A | 0.066 | 0.00 | 45 | — | — | — | >200 |
| 3482 | 0.051 | 0.00 | 0 | — | — | 0 | >200 |
| 3486A | 0.039 | 0.00 | 41 | — | — | — | — |
| 3487A | 0.032 | 0.00 | 26 | — | — | 0 | >200 |
| 3846 | 0.016 | 7.3 | 33 | — | — | 34 | 50 |
| 3847 | 0.008 | 5.4 | 10 | — | — | 34 | 50 |
| 3907 | 0.034 | 0.00 | 9 | — | — | — | 6.25 |
| 3925 | 0.086 | 2.1 | — | — | — | — | 25 |
| 3927 | 0.044 | — | — | — | — | — | 12.5 |
| 3976 | 0.007 | 8.9 | 39 | 0 | 96 | — | 50 |
| 4039 | 0.052 | 0.00 | 51 | — | — | — | — |
| 4054 | 0.17 | 17.3 | 84 | 9 | 97 | — | 25 |
| 4055 | 0.042 | 7.8 | 47 | 5 | 94 | 0 | 25 |
| 4275 | 0.017 | 0.9 | — | — | — | — | >400 |
| 4501 | 0.032 | 13.2 | 85 | — | — | — | 100 |
| 4502 | 0.025 | 13.2 | 28 | 0 | 99 | 96 | 100 |
| 4829 | 0.006 | 0.46 | 96 | 0 | 96 | — | 50 |
| 4830 | 0.01 | — | — | — | — | — | — |
| 4831 | 0.013 | — | — | — | — | — | — |
| 4874 | 0.008 | 8.8 | 96 | 0 | 96 | — | >200 |
| 5068 | 0.083 | — | — | — | — | — | — |
| 5071 | 0.016 | — | — | — | — | — | — |
| 5072 | 0.006 | 2.3 | 90 | 5 | 78 | — | 50 |
| 5073 | 0.008 | 1.4 | 95 | 3 | 84 | — | 50 |

^{a}Pf growth inhibition in PGIM;
^{b}Caco-2 permeability, Pₐₚₚ × 10⁻⁶ cm/sec;
^{c}stability against murine liver microsomes, % consumed after 30 min.;
^{d}stability against mouse serum exposure, % consumed after 60 minutes;
^{e}percent protein bound in mouse serum by equilibrium dialysis;
^{f}cyp 3A4 inhibition at 5 μM;
^{g}solubility limit in H₂O.

### Example 6. Murine Studies

i. Murine Tolerability

Pyridazinone compounds MBX 3318, MBX 3976, and 4055 were administered to CD-1 mice by the intravenous (IV) and oral (PO) routes (3318) and by the PO and intraperitoneal (IP) routes (3976 and 4055) as single doses of 10-200 mg/kg. Animals were monitored for 24 hours for signs of toxicity. For IV dosing of MBX 3318, no symptoms were observed at doses up to 50 mg/kg (data not shown) and the formulation comprising 80% PEG-300, 10% DMSO was deemed acceptable.

For PO dosing (Table 8), all symptoms were considered mild and had resolved by the end of the study; the formulation comprising 80% PEG-300, 5% cremophor, 5% DMSO was considered a factor in these results. For PO dosing of MBX 3976 and 4055, some slight lethargy was observed that was not dose-related (Table 8). All symptoms were considered mild and were considered formulation-related which comprised 10% DMSO, 80% PEG-400, 1% Polysorbate 80. This formulation was more toxic to the mice when administered by the IP route (data not shown).

TABLE 8
Murine tolerability of three pyridazinone compounds (PO)

| Compound; | |
|---|---|
| Dose (mg/kg) | Observations 0-24 hours |
| vehicle ^{a} | Mild irregular breathing, 2 h |
| 3318 ^{a}; (40) | Decreased activity, irregular breathing, 2 h |
| 3318 ^{a}; (100) | Decreased activity, irregular breathing, rough |
| | hair coat, 2 h |
| 3318 ^{a}; (200) | Decreased activity, irregular breathing, rough |
| | hair coat, 2 h |
| vehicle ^{b} | 1 mouse, slightly lethargic, 8 h |
| 3976 ^{b}; (10), (30) | None reported |
| 3976 ^{b}; (65) | 1 mouse, slightly lethargic, 8 h |
| vehicle ^{b} | One mouse, slightly lethargic, 24 h |
| 4055^{b}; (10), (30), (100) | None reported |

Formulations:
^{a} 5% DMSO, 5% cremophor, 80% PEG-300;
^{b} 10% DMSO, 80% PEG-400, 1% Polysorbate 80

ii. Murine Pharmacokinetics

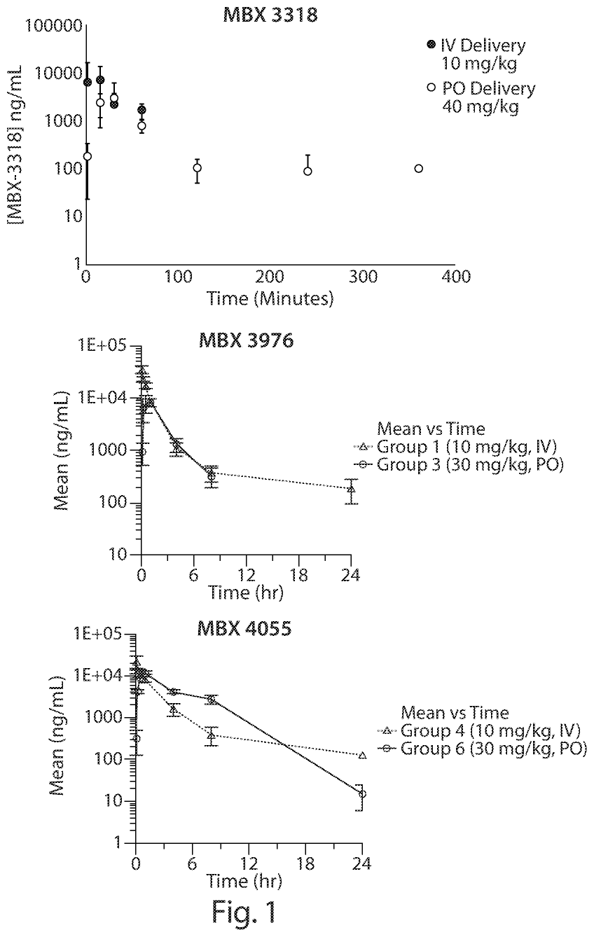

Compounds MBX 3318, 3976, and 4055 were administered to CD-1 mice by the IV and PO routes and serial blood samples were collected at 7 time points (0.083, 0.25, 0.5, 1, 4, 8, 24 hrs). The concentration time curves are shown in FIG. 1. The calculated parameters for all 3 compounds are shown in Table 9.

Overall analysis of the data shows acceptable parameters for MBX 3318 that are dramatically improved for both MBX 3976 and MBX 4055. The half-lives for the 3 compounds ranged from 0.4-2.9 hr, with MBX 3318 the most rapidly cleared. Similarly, IV Cₘₐₓ and AUC values for MBX 3976 and 4055 were much higher than for MBX 3318, despite exhibiting fewer adverse effects in tolerability studies at comparable doses. Finally, oral dosing of the three compounds revealed a >10-fold and >30-fold increase in normalized exposure for MBX 3976 and MBX 4055 relative to MBX 3318, respectively. In fact, MBX 4055 had a much greater oral bioavailability (66%) than did the remaining 2 compounds. These results are significant as MBX 3318 exhibited extremely favorable efficacy despite the relatively low exposures (see below).

TABLE 9
Pharmacokinetic parameters and oral bioavailability

| | MBX 3318^{a} | | MBX 3976^{b} | | MBX 4055^{b} | |
|---|---|---|---|---|---|---|
| Parameter (units) | IV | Oral | IV | Oral | IV | Oral |
| Dose (mg/kg) | 10 | 40 | 10 | 30 | 10 | 30 |
| Half-life (hr) | 0.4 | 1.52 | 2.88 | 1.54 | 1.39 | 2.27 |
| Cₘₐₓ (ng/mL) | 7,437 | 3,064 | 44,454 | 9,520 | 28,786 | 11,733 |
| Tₘₐₓ (hr) | 0.25 | 0.5 | 0.083 | 0.667 | 0.083 | 1 |
| Clearance (mL/hr/kg) | 2,538 | — | 257 | — | 392 | — |
| V_{D} (mL/kg) | 1,344 | — | 1,954 | — | 786 | — |
| Mean residence time (hr) | 0.33 | — | 1.95 | — | 1.35 | — |
| AUCₗₐₛₜ (hr*ng/mL) | 3,941 | 2,826 | 41,059 | 24,277 | 34,275 | 67,501 |
| Normalized AUC | 3,941 | 707 | 41,059 | 8,092 | 34,275 | 22,500 |
| % F (oral bioavailability | — | 18% | — | 20% | — | 66% |

Formulations:
^{a}10% DMSO, 80% PEG400,
^{b}80% PEG400, 1% Polysorbate 80

iii. Murine Efficacy

MBX 3318 was tested in the NSG (humanized) mouse model. This is a critical assay for this compound because pyridazinone compounds do not inhibit the Plasmodium berghei PSAC channel; this murine parasite is used in the standard mouse malaria model.

Humanized NSG mice infected with P. falciparum were treated with 100 mg/kg of MBX 3318 twice daily for 4 days by the PO route. Control mice were treated with vehicle alone (10% DMSO, 80% PEG400, 1% Polysorbate 80). The results are shown in FIG. 2A. As shown in FIG. 2A, MBX 3318 was able to reduce parasite growth by about 73.3% in humanized mice with respect to control animals after 4 days of administration. During the assay, blood samples were taken from mice treated with MBX 3318 to measure the levels of compound (FIG. 2B). The levels were well above the IC₅₀ in vitro of this compound, which should be sufficient for the compound to show inhibitory effects on the parasite. Additionally, the blood levels were only slightly above the IC₅₀ of MBX 3318 tested in standard RPMI medium, suggesting that the PGIM IC₅₀ value is more relevant to murine (and presumably human) serum conditions. This proof-of-concept study is very promising, demonstrating that growth of P. falciparum parasites in mice can be inhibited by PSAC inhibitors.

The murine model was also employed against MBX 4055. Humanized NSG mice infected with P. falciparum were treated with 25 or 50 mg/kg of MBX 4055 twice daily or 50 mg/kg once daily, for 4 days by the PO route. Control mice were treated with vehicle alone (6.25% DMSO, 12.5% polysorbate 80, 43.75% Labrasol, 1.25% hydroxypropyl methylcellulose in H₂O). The results are shown in FIG. 3. As shown in FIG. 3A, MBX 4055 dosed at 50 mg/kg once daily was able to reduce parasite growth by over 90% in a humanized mouse with respect to control mice after 4 days of administration. Unfortunately, the remaining mice that were dosed twice daily, including the vehicle-only mouse died within 2-5 days due to the apparent toxicity of the Labrasol-containing vehicle. During the assay, blood samples were taken from mice treated with MBX 4055 to measure the levels of compound (FIG. 3B). The levels were well above the IC₅₀ in vitro of this compound, which should be sufficient for the compound to show inhibitory effects on the parasite. This proof-of-concept study shows even more promise than for MBX 3318, demonstrating that growth of P. falciparum parasites in mice can be inhibited by PSAC inhibitors.

All publications, patent applications, patents, and other documents cited herein are incorporated by reference in their entirety. In case of conflict, the present specification, including definitions, will control. In addition, the materials, methods, and examples are illustrative only and not intended to be limiting.

Obvious variations to the disclosed compounds and alternative embodiments of the invention will be apparent to those skilled in the art in view of the foregoing disclosure. All such obvious variants and alternatives are considered to be within the scope of the invention as described herein.

1-22. (canceled)

23. A compound of Formula I: 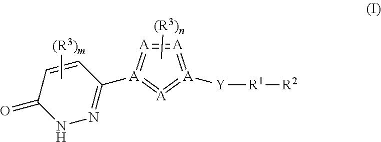
wherein:
A is independently selected from C, S, O or N combined through either single or double bonds to form a five-member heteroaromatic ring of 1-4 carbon atoms, 0-3 nitrogen atoms, 0-1 oxygen atom, and 0-1 sulfur atom;
R³ is a monovalent substituent group independently selected from alkenyl, alkoxy, alkyl, alkynal, having from 1 to 12 carbons, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halo, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent group is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, said substituent group may be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
n is an integer from 0-3;
m is an integer from 0-3;
Y is a divalent radical bridging A and R¹ selected from the group comprising, —COCH₂—, —SO₂—, —CO—, —CH₂—, —CH(CH₃)—, —NHCO—, —NCH₃CO—, —CONH—, —CONCH₃—, —O(CO)—, —(CO)O—, —NH—, or —O—;
R¹ is a divalent non-aromatic, heterocyclic ring of 5-7 members containing 0-2 nitrogen atoms, 0-1 oxygen atom, and 3-6 carbon atoms, with the proviso that Y and R² are separated by at least 3 atoms, which non-aromatic, heterocyclic ring may bear 0-3 substituent groups defined as for R³, with the proviso that two or more such substituent groups on R¹ may be fused with R¹ to form one or more cycloalkyl, heterocyclic, aromatic, or heteroaromatic rings, or alternatively R¹ may be fused, optionally incorporating 0-2 substituent groups, with R² to form a fused heterocyclyl ring of 3-7 members, optionally substituted with 0-2 substituent groups defined as for R³;
R² is a 5- or 6-membered heteroaryl ring bearing 0-4 substituent groups independently selected from substituent groups defined as for R³, or substituents on R² may be optionally fused to R² to form one or more cycloalkyl, heterocyclic, aryl or heteroaryl rings, or 0 -2 R² substituents may, together with R¹, form a fused substituted or unsubstituted heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
or a pharmaceutically acceptable salt thereof.

24. The compound according to claim 23, wherein said compound has the structure of Formula I(a): 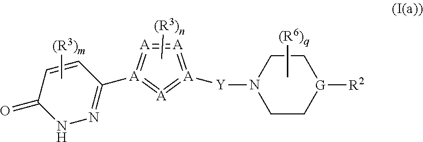
wherein:
G is selected from C or N and is part of a heterocyclic ring which is optionally substituted with (R⁶)_{q}, where q is an integer from 0-4; and
R⁶ is as defined for R³, with the additional proviso that R⁶ substituents on the heterocyclic ring containing G may be optionally fused to each other or a carbon atom of the ring containing G to form one or more cycloalkyl, heterocyclic, aromatic, or heteroaromatic rings; or 0-2 substituents on the heterocyclic ring containing G may, together with R², form a fused substituted or unsubstituted cycloalkyl or heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
R² is a 5- or 6-membered heteroaryl ring bearing 0-4 substituents independently selected from substituent groups defined as for R³, or substituents on R² may be optionally fused to R² to form one or more cycloalkyl, heterocyclic, aryl, or heteroaryl rings, of 3-8 members;
or a pharmaceutically acceptable salt thereof.

25. A compound according to claim 24, wherein said compound has the structure of Formula I(b): 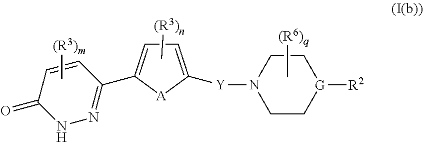
wherein:
A is independently selected from O, S or N, wherein,
n is an integer from 0-2 when A is O or S, and
n is an integer from 0-3 when A is N;
or a pharmaceutically acceptable salt thereof.

26. The compound according to claim 23, wherein said compound is selected from the group consisting of:

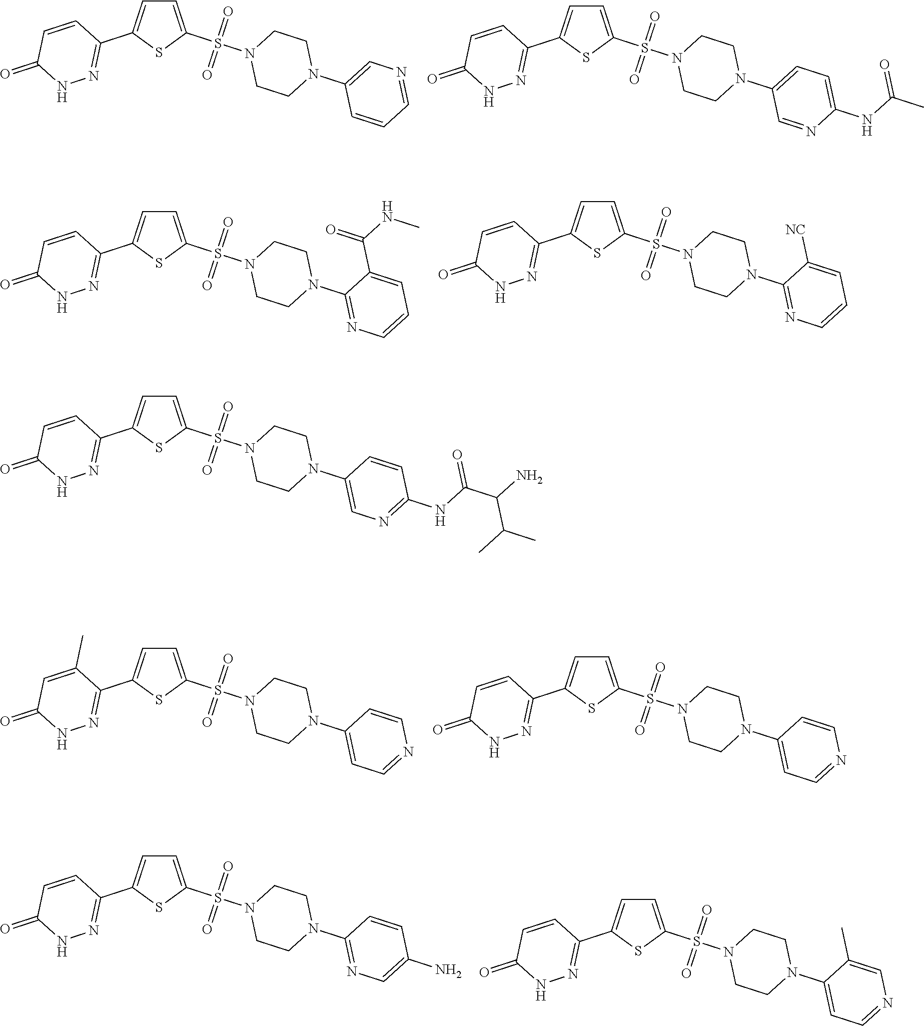

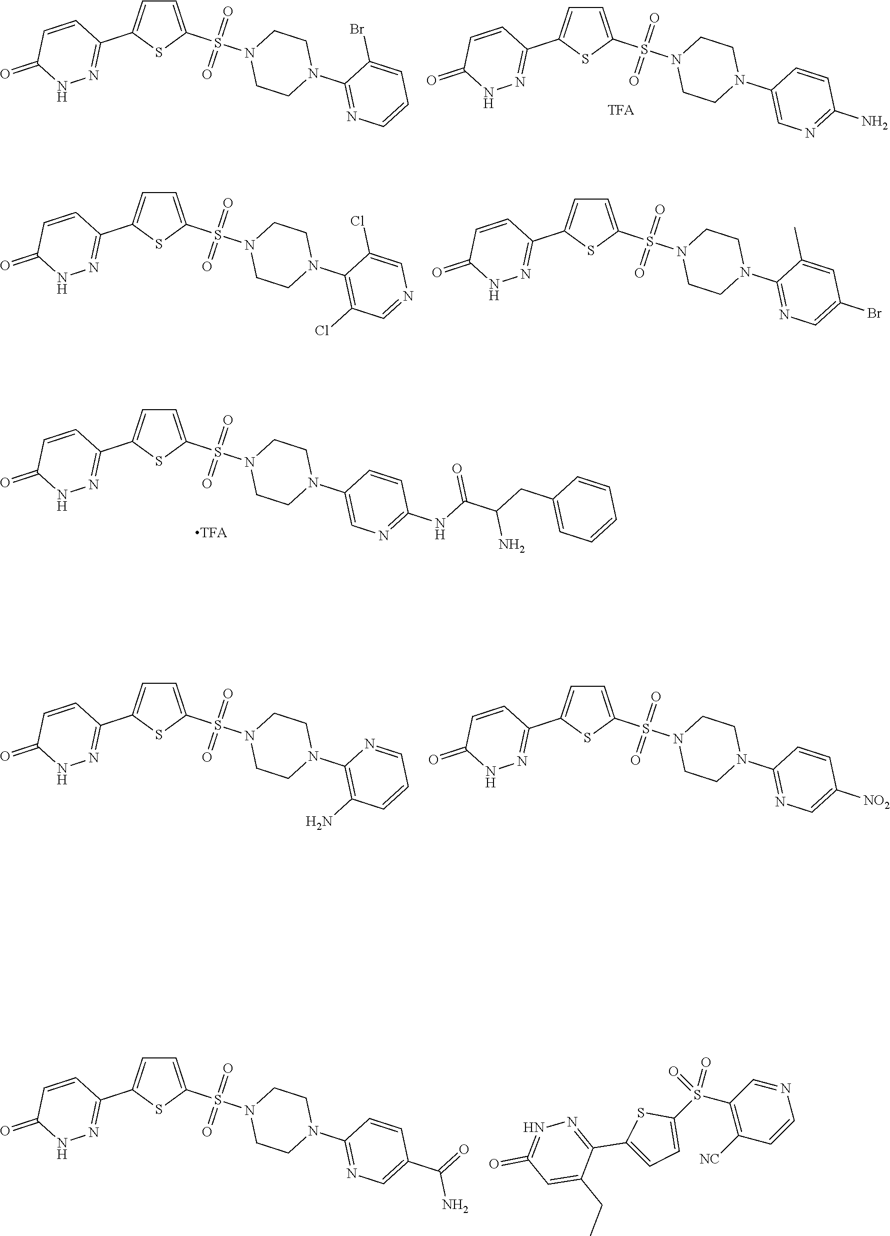

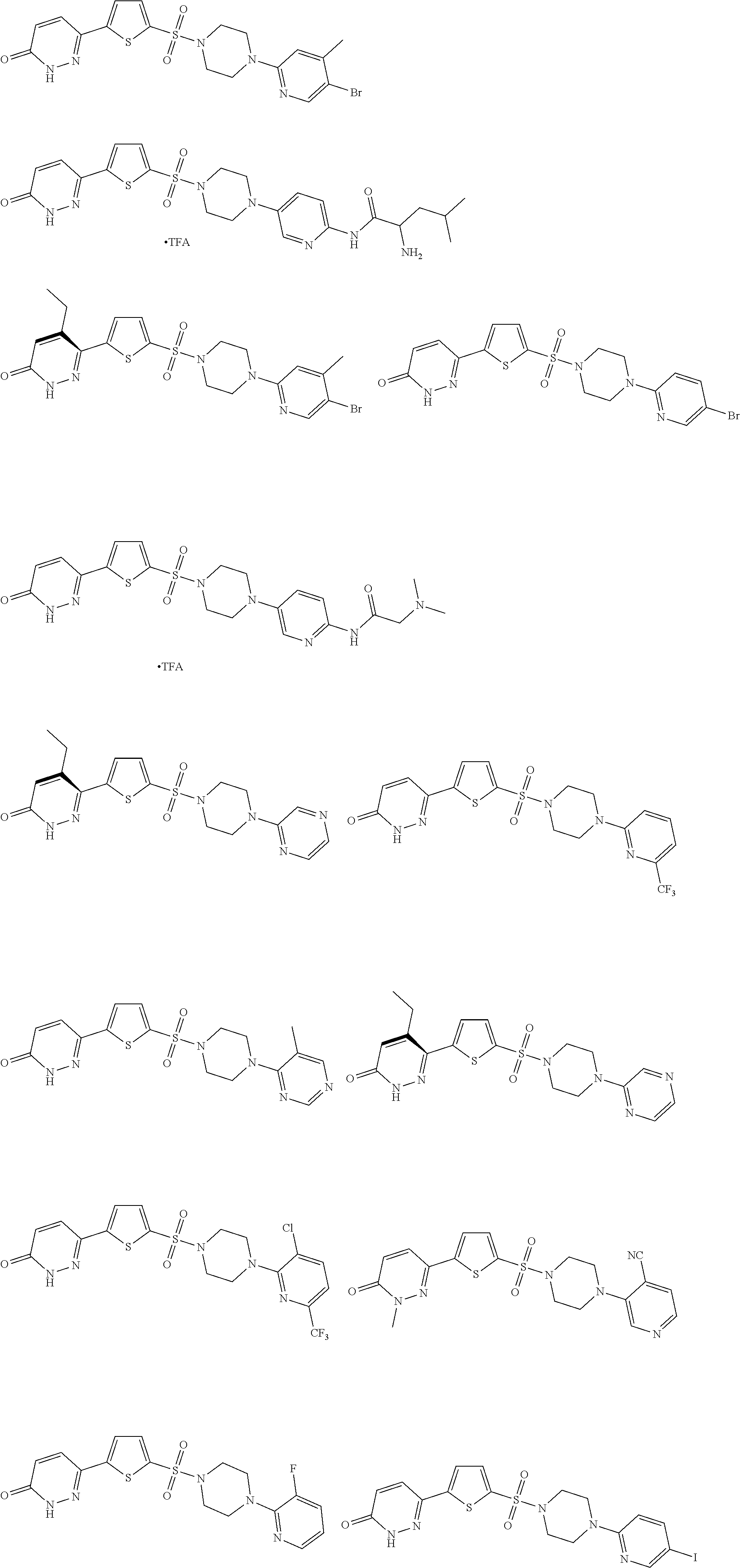

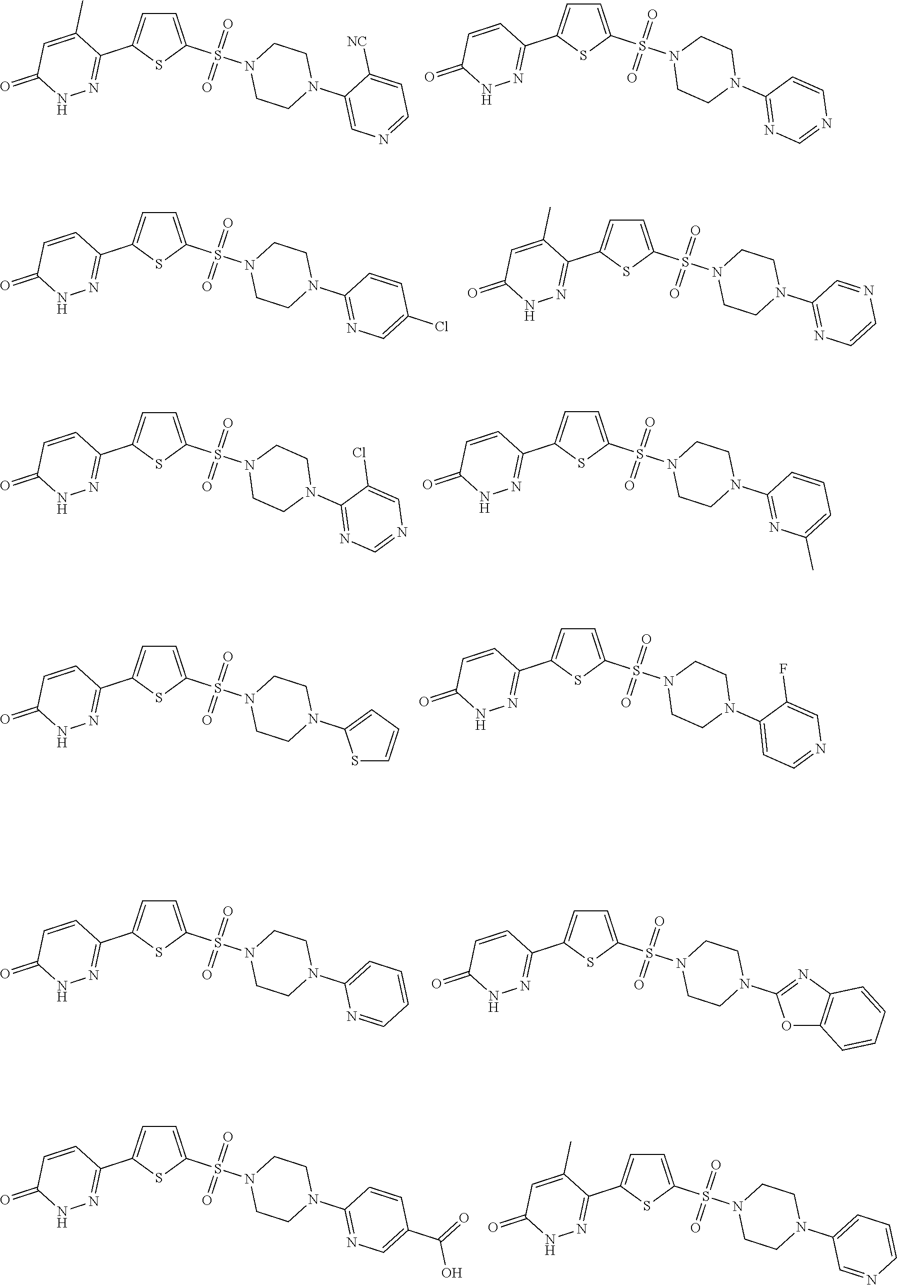

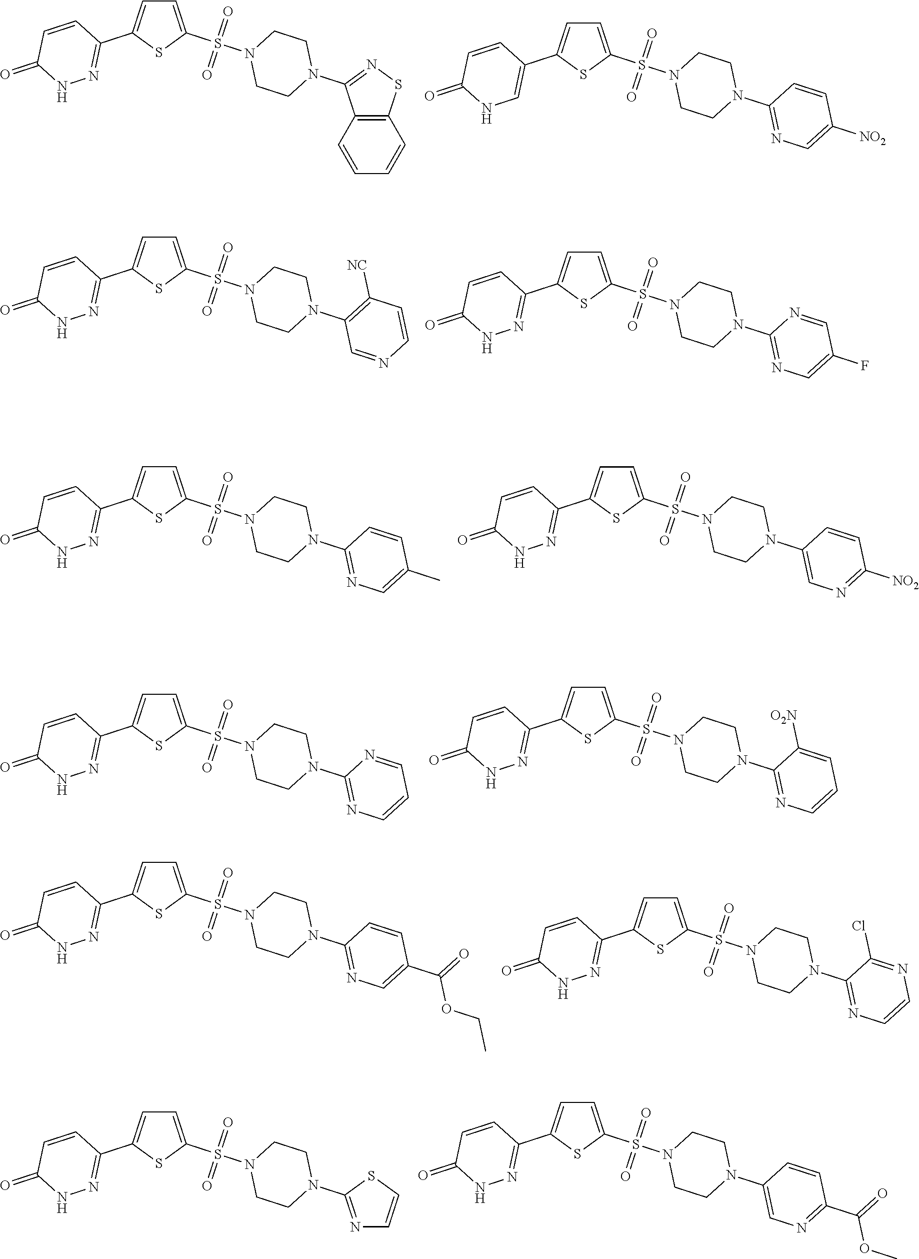

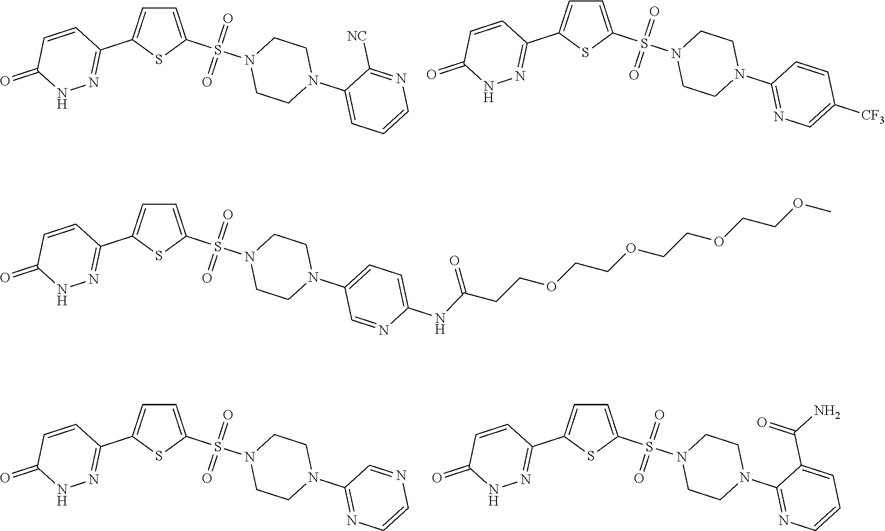

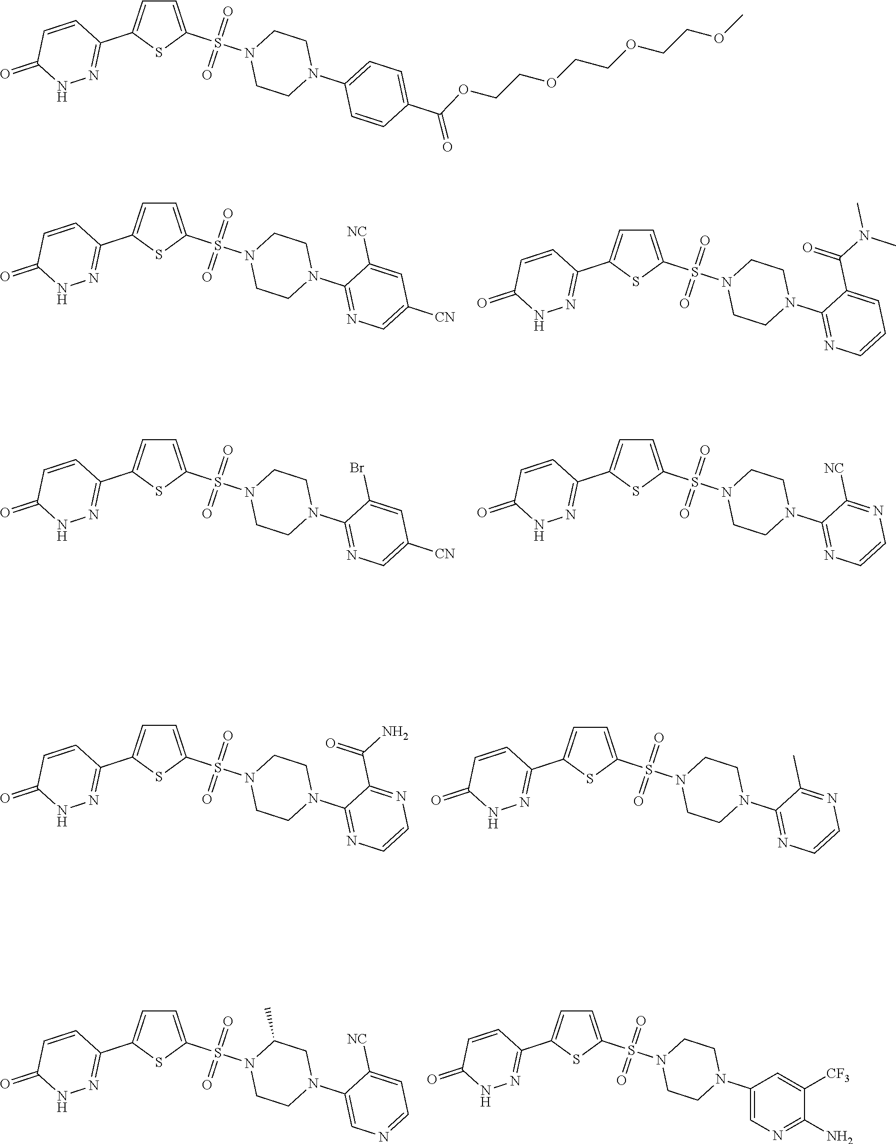

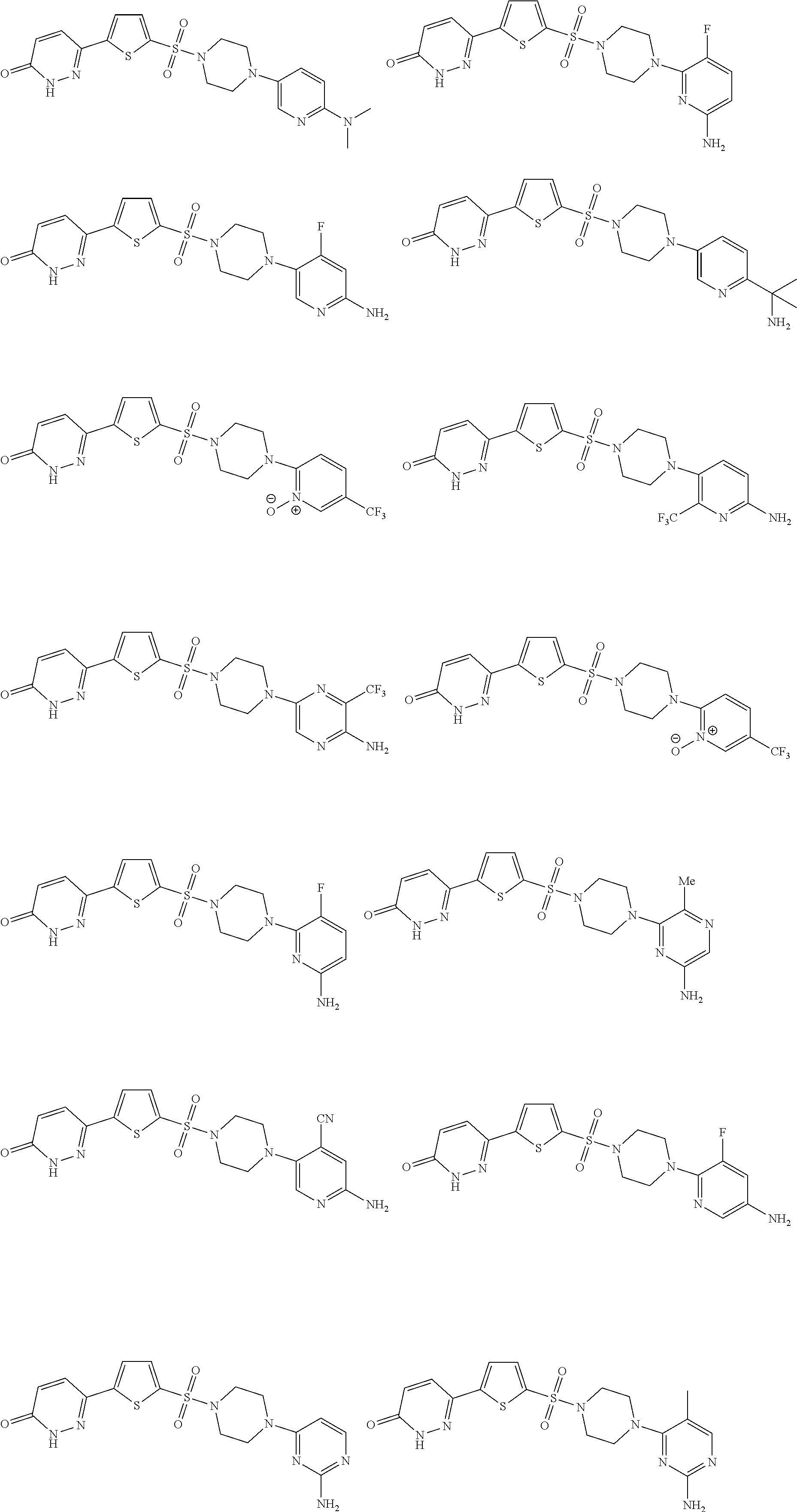

27. A method for treating or preventing malaria in a mammalian subject comprising administering an effective amount of a compound according to any one of claims 23.

28. The method according to claim 27, wherein said mammal is a human.

29. The method according to claim 27, further comprising administering a second antimalarial agent.

30. The method according to claim 27, wherein the compound is formulated for administration in a pharmaceutically acceptable carrier or excipient.

31. A pharmaceutical composition for treating or preventing malaria in a mammalian subject, said composition comprising a compound of Formula II:
Q-Y—R¹—R² (II)
wherein:
Q is a heteroaryl ring of 5 members having group Y bound to the ring at a non-adjacent site to a 6-pyridazin-3-(2H)-one, 5-pyridin-2(1H)-one, a substituted carboxamide, or a substituted carboxylate moiety, wherein Q is optionally substituted on either the heteroaryl ring, the 6-pyridazin-3-(2H)-one moiety, or both, with one or more substituent groups independently selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, and thiocarbonyl, each substituent group may be optionally substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, and thiol;
Y is a divalent radical bridging Q and R¹ selected from the group comprising: —COCH₂—, —CH₂CO—, —SO₂—, —CO—, —CH₂—, —CH(CH₃)—, —NHCO—, —NCH₃CO—, —CONH—, —CONCH₃—, —O(CO)—, —(CO)O—, —NH—, and —O—;
R¹ is a divalent non-aromatic heterocyclic ring of between 5-7 members containing 0-2 nitrogen atoms, 0-1 oxygen atoms, and 3-6 carbon atoms, with the proviso that Y and R² are separated by at least 3 atoms, which non-aromatic, heterocyclic ring may bear 0-3 substituent groups selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when the substituent group is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, each substituent can be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halo, heteroaryl, heterocyclyl, hydroxyl, imino, nitro, phosphate, sulfinyl, sulfonamidyl, sulfonyl, thioalkyl, thioaryl, thiocarbonyl, thioether, or thiol, with the proviso that two or more such substituent groups on R¹ may be fused with R¹ to form one or more cycloalkyl or heterocyclic rings, or alternatively R¹ may be fused with R² to form a fused cycloalkyl or heterocyclyl ring of 3-7 members, optionally substituted with 0-2 substituent groups selected from alkenoxy, alkenyl, alkoxy, alkyl, alkylamino, alkynal, alkynoxy, amide, amidino, amino, aminoalkyl, aminoaryl, aryl, arylalkyl, aryloxy, azido, azo, carbamate, carbamide, carbonyl, carboxamide, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; and
R² is a 5- or 6-membered heteroaryl ring bearing 0-4 substituent groups independently selected from alkenyl, alkoxy, alkyl, alkynal, amido, amidino, amino, aminoalkyl, aminoaryl, aryl, aryloxy, azido, azo, carbamate, carbamide, carbonyl, carboxamido, carboxylate, cyano, cycloalkyl, ester, guanidino, halogen, heteroaryl, heterocyclyl, hydroxyl, imino, nitro, phosphate, sulfinyl, sulfonamidyl, sulfonyl, thioalkyl, thioaryl, thiocarbonyl, or thiol, and, when said substituent is alkenyl, alkoxy, alkyl, alkynal, amido, amidino, aminoalkyl, aminoaryl, aryl, aryloxy, carbamate, carbamide, carbonyl, carboxamido, carboxylate, cycloalkyl, ester, guanidino, heteroaryl, heterocyclyl, imino, phosphate, sulfinyl, sulfonamidyl, sulfonyl, thioalkyl, thioaryl, or thiocarbonyl, said substituent group may be further substituted with 0-3 groups independently selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidino, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol; or
substituents on R² may be optionally fused to R² to form one or more cycloalkyl, heterocyclic, aryl or heteroaryl rings; or 0-2 R² substituents may, together with R¹, form a fused substituted or unsubstituted cycloalkyl or heterocyclyl ring bearing 0-2 additional substituents selected from alkenoxy, alkenyl, alkoxy, alkyl, alkylamino, alkynal, alkynoxy, amido, amidino, amino, aminoalkyl, aminoaryl, aryl, arylalkyl, aryloxy, azido, azo, carbamate, carbamide, carbonyl, carboxamido, carboxylate, cyano, cycloalkyl, ester, ether, guanidine, haloalkoxy, haloalkyl, halogen, heteroaryl, heterocyclyl, hydroxyl, imino, nitro, phosphate, sulfinyl, sulfonamidyl, sulfonyl, thioalkyl, thioaryl, thiocarbonyl, thioether, or thiol;
or a pharmaceutically acceptable salt thereof.

32. The composition according to claim 31, wherein said compound is selected from the group consisting of:

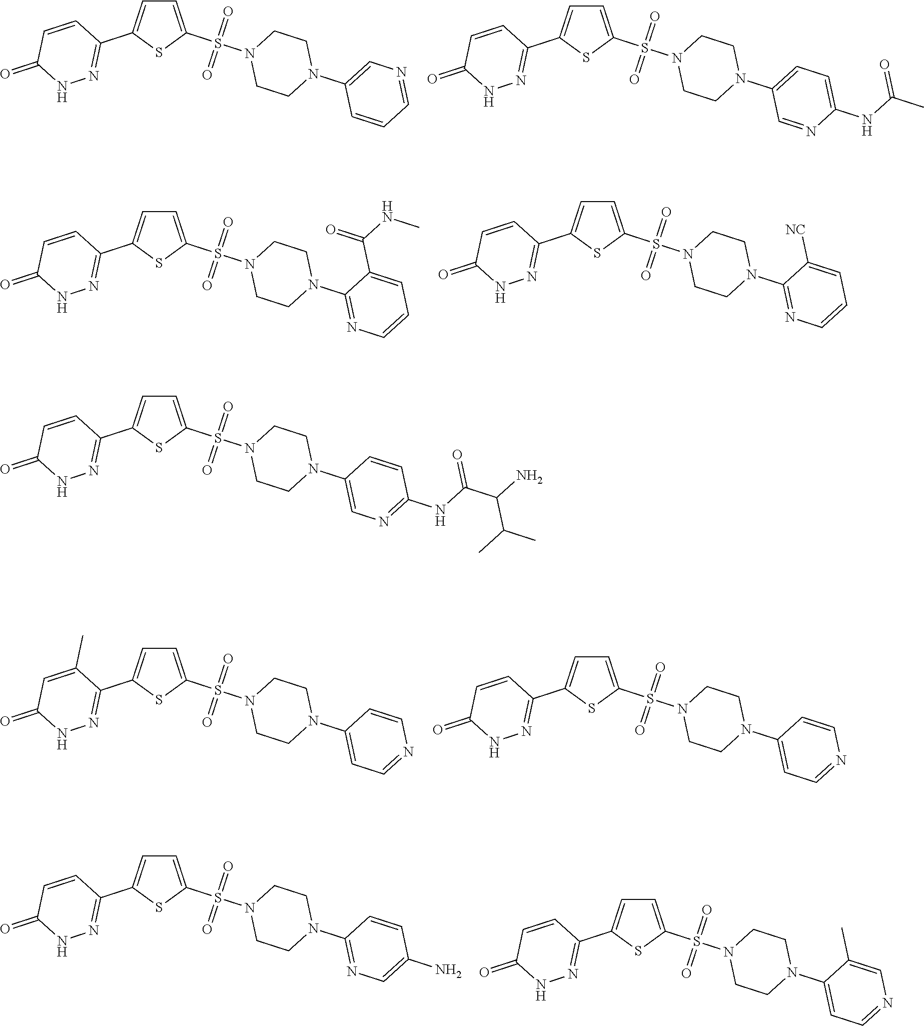

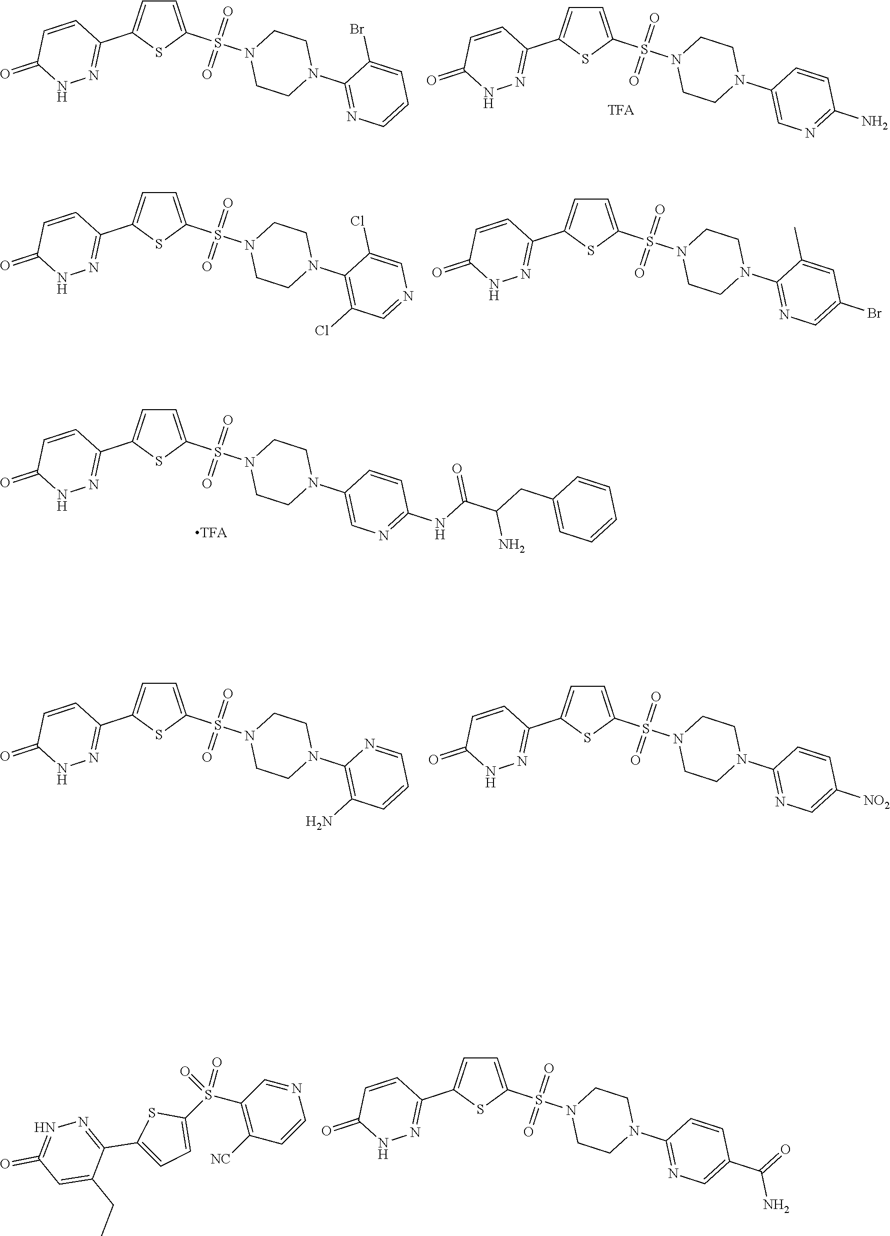

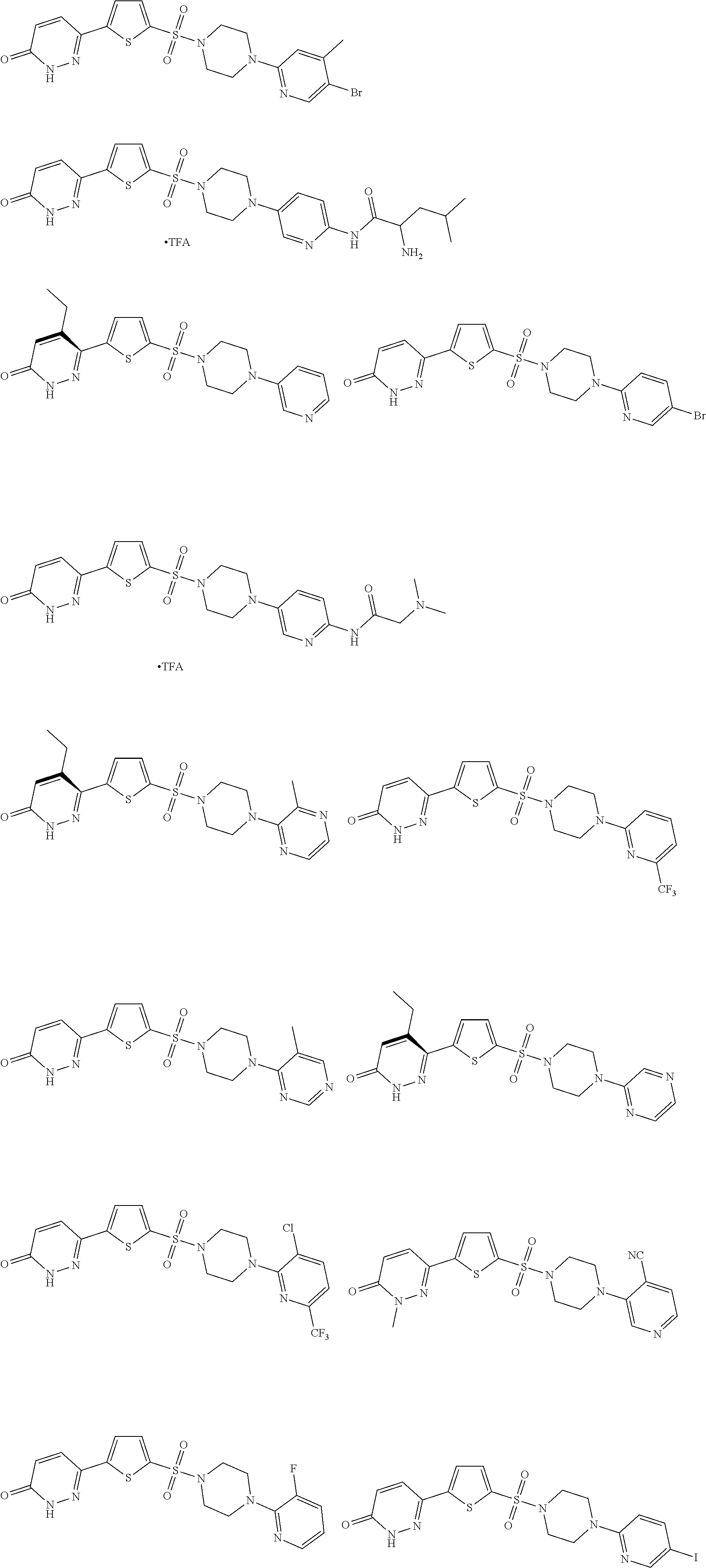

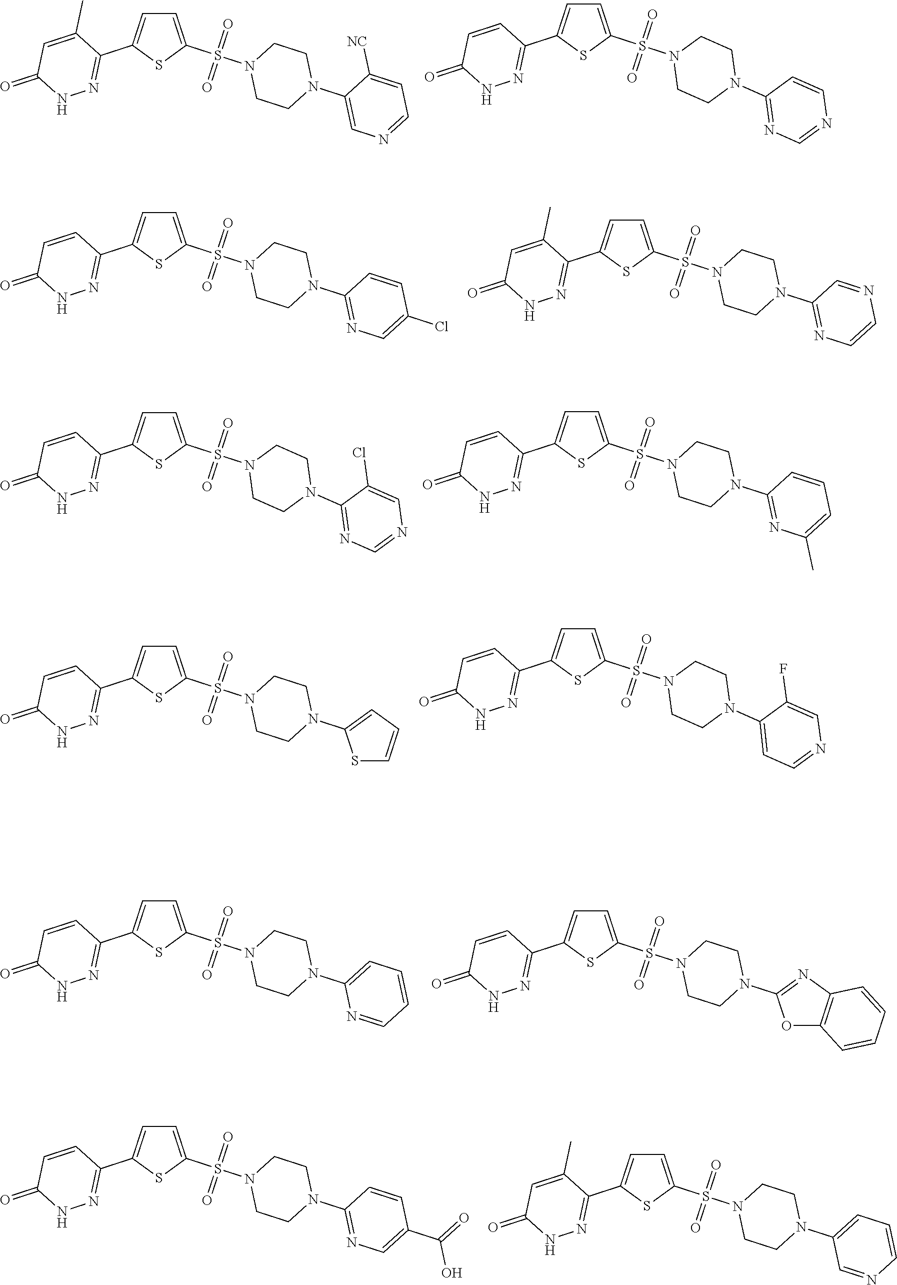

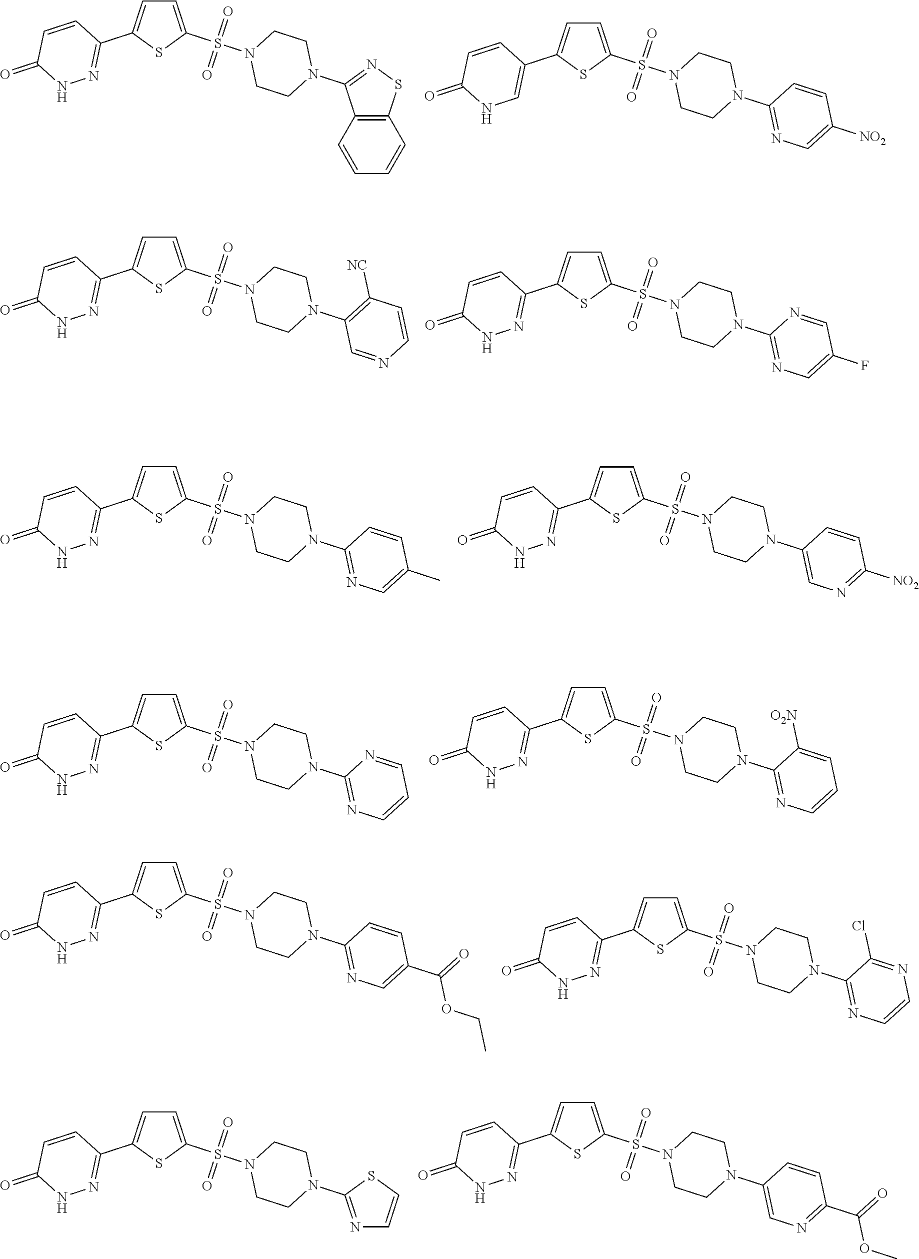

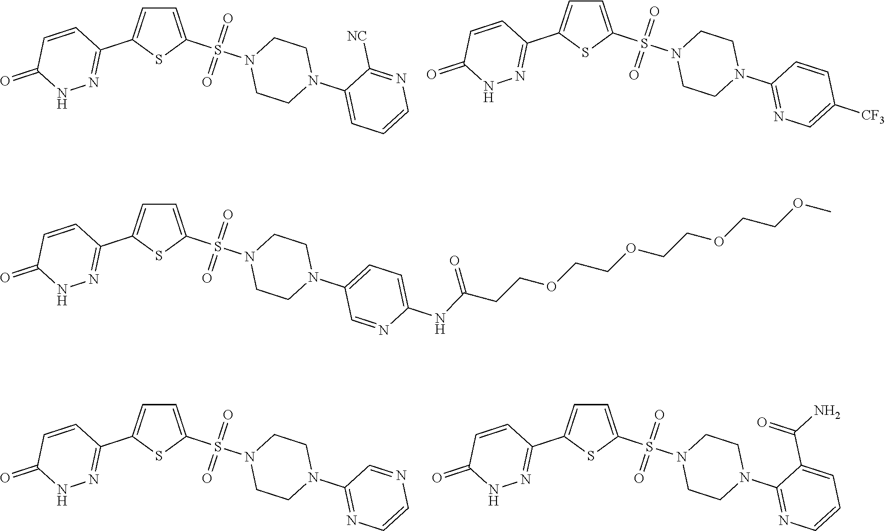

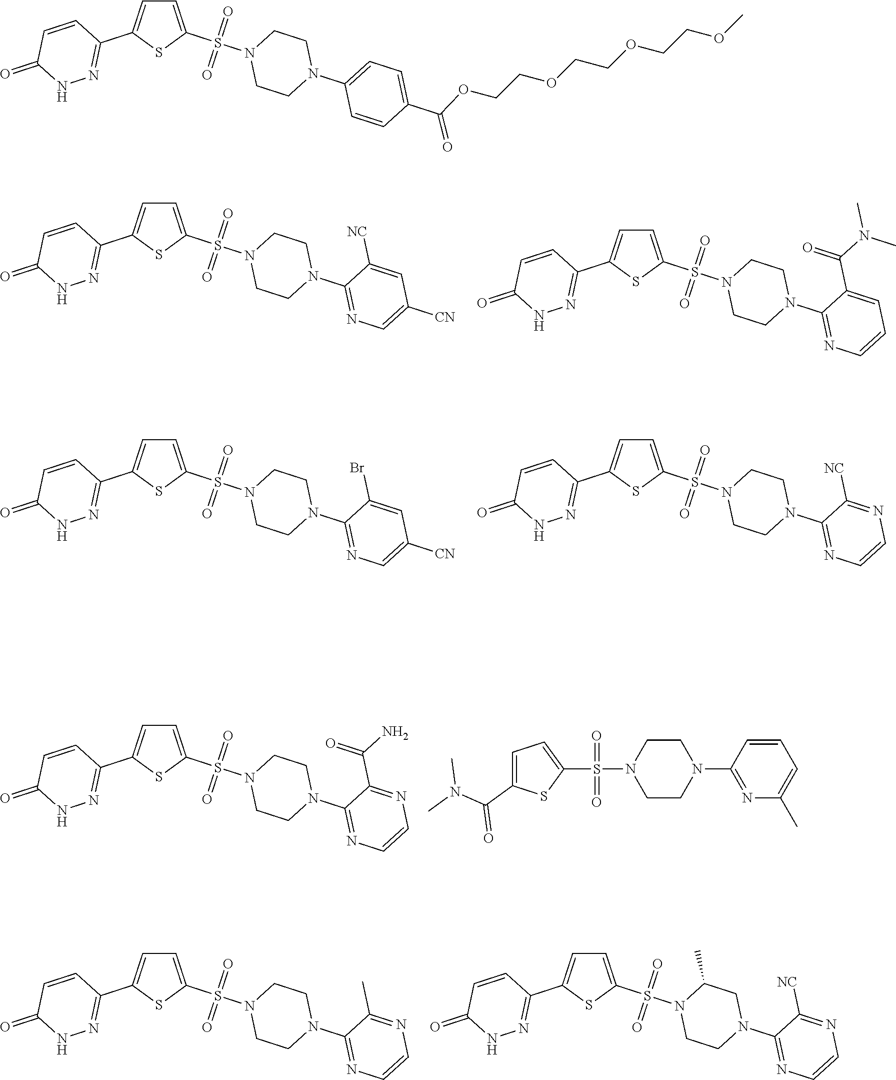

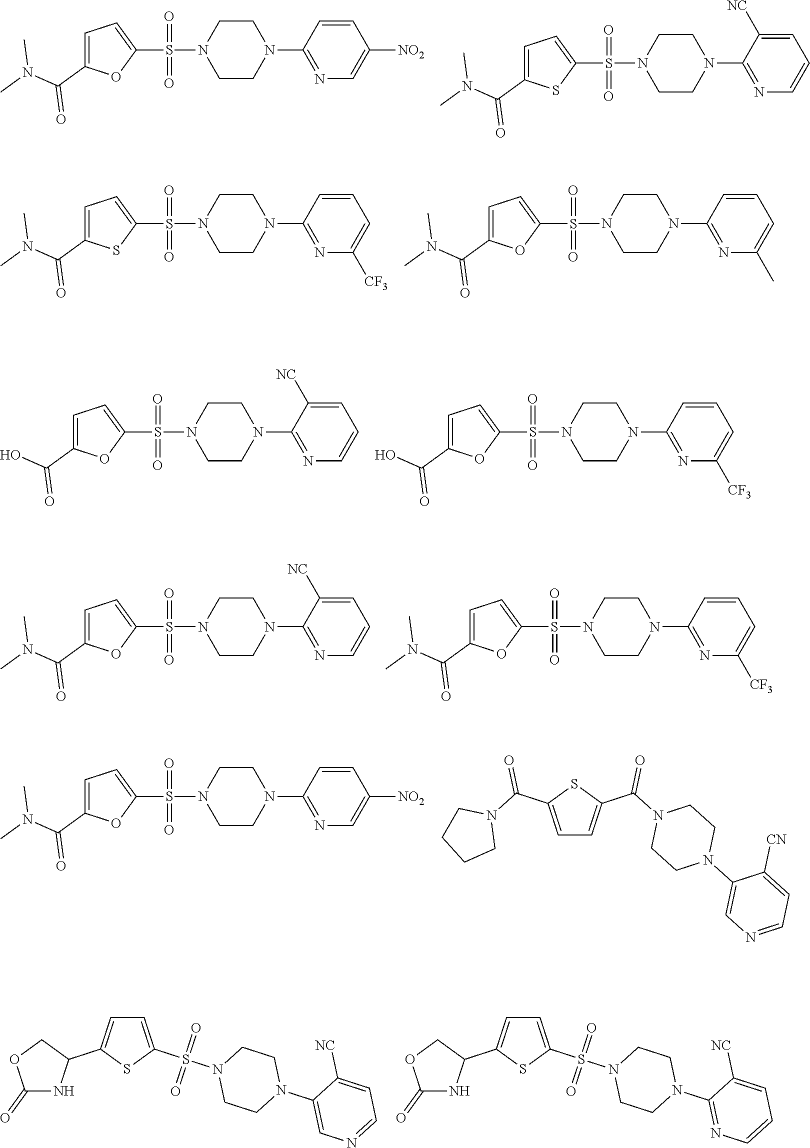

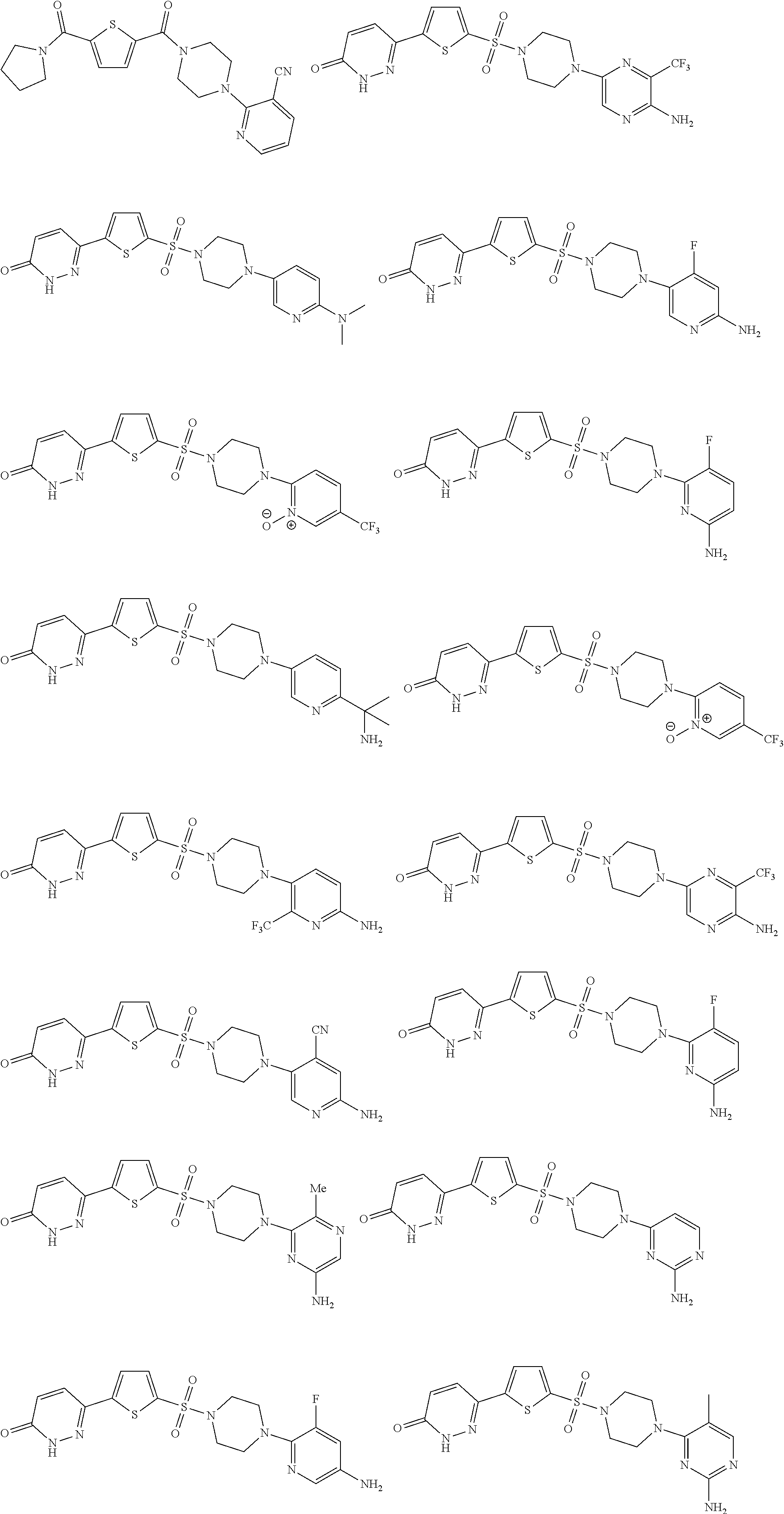

33. A method for treating or preventing malaria in a mammalian subject comprising administering to said subject an effective amount of a composition according to claim 31.

34. The method according to claim 33, wherein said mammalian subject is a human.

35. The method according to claim 33, further comprising administering a second antimalarial agent.

36. The method according to claim 33, wherein the composition is formulated using a pharmaceutically acceptable carrier or excipient.
